(19) United States
(12) Patent Application Publication
(10) Pub. No.: US 2024/0197915 A1
(43) Pub. Date: Jun. 20, 2024

(54) METHODS OF TREATING OCULAR DISEASES USING AAV2 VARIANTS ENCODING AFLIBERCEPT

(71) Applicant: Adverum Biotechnologies, Inc., Redwood City, CA (US)

(72) Inventors: Julie CLARK, Santa Monica, CA (US); Scott WHITCUP, Irvine, CA (US); Adam TURPCU, Oakland, CA (US)

(21) Appl. No.: 18/554,965

(22) PCT Filed: Apr. 26, 2022
(86) PCT No.: PCT/US2022/071937

§ 371 Date: Oct. 11, 2023

Related U.S. Application Data
(60) Provisional application No. 63/180,646, filed on Apr. 27, 2021.

(51) Int. Cl. A61K 48/00; A61K 31/573; A61K 38/16; A61K 38/17; A61K 47/02; A61K 47/10; C12N 15/62; C12N 15/86

(52) U.S. Cl. CPC A61K 48/005; A61K 31/573; A61K 38/162; A61K 38/179; A61K 47/02; A61K 47/10; A61K 48/0075; A61K 48/0083; C12N 15/62; C12N 15/86; C12N 2750/14122; C12N 2750/14133; C12N 2750/14143

(57) ABSTRACT

Provided are methods for treating an ocular disease in an individual, comprising administering a unit dose of recombinant adeno-associated virus (rAAV) particles to an eye of the individual, wherein the rAAV particles comprise: a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

### CROSS-REFERENCE TO RELATED APPLICATIONS

This application claims the benefit of and the priority to U.S. Provisional Patent Application No. 63/180,646 filed Apr. 27, 2021, the disclosure of each of which are hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 627002001340SEQLIST.TXT, date recorded: Apr. 26, 2022, size: 41,493 bytes).

### FIELD

The present disclosure relates to methods of treating ocular diseases and disorders in an individual that comprise administering a single unit dose of a recombinant adeno associated virus (rAAV) particles encoding an anti-VEGF agent (e.g., aflibercept) to an eye of an individual.

### BACKGROUND

Aflibercept is a recombinant fusion protein that acts as a decoy receptor for vascular endothelial growth factor subtypes A and B (VEGF-A and VEGF-B) and placental growth factor (PGF). By binding to these ligands, aflibercept is able to prevent them from binding to vascular endothelial growth factor receptors (VEGFR), VEGFR-1 and VEGFR-2, to, inter alia, suppress neovascularization and decrease vascular permeability. Aflibercept consists of domain 2 of VEGFR-1 and domain 3 of VEGFR-2 fused with the Fc fragment of IgG1.

Current standard of care anti-VEGF agents such as aflibercept need to be re-administered via intravitreal (IVT) injection every 4 to 8 weeks to achieve optimal therapeutic outcomes and maintain visual acuity. Compliance with such a regimen is burdensome to patients, their caregivers, and the healthcare system, and most patients fall out of compliance with the optimal regimen over time, which is correlated with vision loss (Khanani A M, et al.). In addition, there are complications including endophthalmitis, retinal detachments, traumatic cataract, and elevated intraocular pressure (IOP); the risks of these complications are likely to increase with repeated IVT injections (Falavarjani et al., (2013) Eye (Lond), 27(7):787-794).

Therefore, there is a need in the art for therapies for ocular diseases that are effective, reduce the risk of adverse effects, and are amenable to high long-term patient compliance.

Use of AAV encoding aflibercept for treating eye disorders is found in U.S. patent application Ser. No. 17/017,469, WO 2021/050094, and WO 2021/050649, incorporated by reference in their entireties.

### SUMMARY OF THE DISCLOSURE

In some aspects, the invention provides a method for treating glaucoma in an individual, the method comprising administering a unit dose of recombinant adeno-associated virus (rAAV) particles to one eye of the individual, wherein the individual is a human, and wherein the rAAV particles comprise a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. In some embodiments, the glaucoma is neovascular glaucoma. In some aspects, the invention provides a method for reducing intraocular pressure in an individual, the method comprising administering a unit dose of recombinant adeno-associated virus (rAAV) particles to one eye of the individual, wherein the individual is a human, and wherein the rAAV particles comprise a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. In some embodiments, the individual has glaucoma. In some embodiments, the glaucoma is neovascular glaucoma.

In some embodiments of the invention, the unit dose of rAAV particles is about 6×10¹¹ vector genomes per eye (vg/eye) or less. In some embodiments, the unit dose of rAAV particles is between about 6×10¹⁰ to about 6×10¹¹ vector genomes per eye (vg/eye). In some embodiments, the unit dose of rAAV particles is between about 6×10¹⁰ to about 2×10¹¹ vector genomes per eye (vg/eye). In some embodiments, the unit dose of rAAV particles is between about 2×10¹¹ to about 6×10¹¹ vector genomes per eye (vg/eye). In some embodiments, the unit dose of rAAV particles is about 2×10¹¹ or about 6×10¹¹ vector genomes per eye (vg/eye). In some embodiments, the unit dose of rAAV particles is about 2×10¹¹ vector genomes per eye (vg/eye). In some embodiments, the unit dose of rAAV particles is about 6×10¹¹ vector genomes per eye (vg/eye).

In some embodiments, the method further comprises administering a unit dose of rAAV particles to the contralateral eye of the individual. In some embodiments, the administering the unit dose of rAAV particles to the contralateral eye is up to about 2 weeks after administering the unit dose of rAAV particles to the one eye. In some embodiments, the method comprises (a) the administering the unit dose of rAAV particles to the contralateral eye is on the same day as the administering the unit dose of rAAV particles to the one eye; or (b) the administering the unit dose of rAAV particles to the contralateral eye is between about 1 day to about 14 days after administering the unit dose of rAAV particles to the one eye. In some embodiments, the unit dose of rAAV particles administered to the contralateral eye of the individual comprises the same or less vector genomes per eye (vg/eye) than the unit dose of rAAV particles administered to the one eye of the individual. In some embodiments, the administering the unit dose of rAAV particles to the contralateral eye is at least about 2 weeks after administering the unit dose of rAAV particles to the one eye. In some embodiments, the unit dose of rAAV particles administered to the contralateral eye of the individual comprises more vector genomes per eye (vg/eye) than the unit dose of rAAV particles administered to the one eye of the individual.

In some embodiments of the invention, the nucleic acid comprises the nucleic acid sequence of SEQ ID NO: 40 or a sequence having at least 85% identity thereto. In some embodiments, the polypeptide comprises the amino acid sequence of SEQ ID NO: 35. In some embodiments, the polypeptide comprises the amino acid sequence of SEQ ID NO: 41. In some embodiments, the polypeptide is aflibercept.

In some embodiments of the invention, the nucleic acid further comprises a first enhancer region, a promoter region, a 5′UTR region, a second enhancer region, and a polyadenylation site. In some embodiments, the nucleic acid comprises, in the 5′ to 3′ order: (a) a first enhancer region; (b) a promoter region; (c) a 5′UTR region; (d) a nucleic acid encoding a polypeptide comprising an amino acid sequence with at least about 95% identity to the amino acid sequence of SEQ ID NO: 35; (e) a second enhancer region; and (f) a polyadenylation site; and flanked by AAV2 inverted terminal repeats (ITRs). In some embodiments, the first enhancer region comprises a CMV sequence comprising the sequence of SEQ ID NO: 22 or a sequence having at least 85% identity thereto. In some embodiments, the promoter region comprises a CMV sequence comprising the sequence of SEQ ID NO: 23 or a sequence having at least 85% identity thereto. In some embodiments, the nucleic acid encoding a polypeptide comprises the nucleic acid sequence of SEQ ID NO: 40 or a sequence having at least 85% identity thereto. In some embodiments, the polypeptide comprises the amino acid sequence of SEQ ID NO: 35 or a sequence having at least 95% identity thereto. In some embodiments, the polypeptide comprises the amino acid sequence of SEQ ID NO: 41 or a sequence having at least 95% identity thereto. In some embodiments, the polypeptide is aflibercept. In some embodiments, the 5′UTR region comprises, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto. In some embodiments, the second enhancer region comprises a full EES sequence comprising the sequence of SEQ ID NO: 26 or a sequence having at least 85% identity thereto. In some embodiments, the polyadenylation site comprises a HGH polyadenylation site comprising the sequence of SEQ ID NO: 27 or a sequence having at least 85% identity thereto. In some embodiments, the nucleic acid further comprises (a) a first enhancer region comprising a CMV sequence comprising the sequence of SEQ ID NO: 22 or a sequence having at least 85% identity thereto; (b) a promoter region, comprising a CMV sequence comprising the sequence of SEQ ID NO: 23 or a sequence having at least 85% identity thereto; (c) a 5′UTR region comprising, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto; (d) a second enhancer region comprising a full EES sequence comprising the sequence of SEQ ID NO: 26 or a sequence having at least 85% identity thereto; and (e) a HGH polyadenylation site comprising the sequence of SEQ ID NO: 27 or a sequence having at least 85% identity thereto. In some embodiments, the nucleic acid comprises the sequence of SEQ ID NO: 39 or a sequence having at least 85% identity thereto.

In some embodiments of the invention, the AAV2 capsid protein comprises the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the AAV2 capsid protein comprises the amino acid sequence LGETTRP (SEQ ID NO: 14) inserted between positions 587 and 588 of the AAV2 VP1 comprising the sequence of SEQ ID NO: 13. In some embodiments, the AAV2 capsid protein comprises the amino acid sequence LALGETTRPA (SEQ ID NO: 1) inserted between positions 587 and 588 of the AAV2 VP1 comprising the sequence of SEQ ID NO: 13. In some embodiments, the rAAV particles comprise an AAV2 VP1 capsid protein comprising a GH loop that comprises the amino acid sequence of SEQ ID NO: 38 or an amino acid sequence having at least 90% sequence identity to SEQ ID NO: 38.

In some embodiments of the invention, the administration of the unit dose of rAAV particles to the one eye and/or the contralateral eye is by intravitreal administration.

In some embodiments of the invention, the unit dose of rAAV particles is in a pharmaceutical formulation. In some embodiments, the pharmaceutical formulation comprises the rAAV particles, sodium chloride, sodium phosphate and a surfactant. In some embodiments, the pharmaceutical formulation comprises about 150 to about 200 mM sodium chloride, about 1 to about 10 mM monobasic sodium phosphate, about 1 to about 10 mM dibasic sodium phosphate, about 0.0005% (w/v) to about 0.005% (w/v) poloxamer 188, and about 6×10¹³ to about 6×10¹⁰ vector genomes (vg) per mL (vg/mL) of the rAAV particles, wherein the pharmaceutical formulation has a pH of about 7.0 to about 7.5. In some embodiments, the pharmaceutical formulation comprises about 180 mM sodium chloride, about 5 mM monobasic sodium phosphate, about 5 mM dibasic sodium phosphate, about 6×10¹² vg/mL of the rAAV particles, and about 0.001% (w/v) poloxamer 188, wherein the pharmaceutical formulation has a pH of about 7.3. In some embodiments, the pharmaceutical formulation comprises about 180 mM sodium chloride, about 5 mM monobasic sodium phosphate, about 5 mM dibasic sodium phosphate, about 2×10¹² vg/mL of the rAAV particles, and about 0.001% (w/v) poloxamer 188, wherein the pharmaceutical formulation has a pH of about 7.3. In some embodiments, the pharmaceutical formulation comprises about 180 mM sodium chloride, about 5 mM monobasic sodium phosphate, about 5 mM dibasic sodium phosphate, about 6×10¹¹ vg/mL of the rAAV particles, and about 0.001% (w/v) poloxamer 188, wherein the pharmaceutical formulation has a pH of about 7.3.

In some embodiments of the invention, the unit dose of rAAV particles administered to the one eye and/or to the contralateral eye in a volume of about 25 μL to about 250 μL. In some embodiments, the unit dose of rAAV particles administered to the one eye and/or to the contralateral eye comprises a volume of about 100 L. In some embodiments, the unit dose of rAAV particles administered to the one eye and/or to the contralateral eye comprises a volume of about 30 L.

In some embodiments of the invention, the individual received prior treatment for the ocular disease with an anti-VEGF agent. In some embodiments, the individual has received 1 or 2 injections of an anti-VEGF agent in the one eye and/or in the contralateral eye prior to administration of the rAAV particles in the one eye and/or in the contralateral eye. In some embodiments, the anti-VEGF agent is aflibercept.

In some embodiments of the invention, the individual has not received prior treatment for the ocular disease with an anti-VEGF agent. In some embodiments, the unit dose of rAAV particles is administered in combinations with administration of an anti-VEGF agent. In some embodiments, the method comprises administering the unit dose of rAAV particles to the one eye of the individual about 1 week or about 7 days after administration of the anti-VEGF agent. In some embodiments, the method comprises administering the anti-VEGF agent to the one eye of the individual on Day 1, and administering the unit dose of rAAV particles to the one eye of the individual on Day 8. In some embodiments, the anti-VEGF agent comprises aflibercept. In some embodiments, the aflibercept is administered at a dose of about 2 mg by intravitreal injection.

In some embodiments of the invention, the unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is a topical steroid treatment. In some embodiments, the steroid treatment is a difluprednate treatment. In some embodiments, the steroid is administered before, during and/or after administration of the unit dose of rAAV particles to the one eye and/or to the contralateral eye. In some embodiments, the topical steroid comprises difluprednate 0.05% at a dose of about 1 μg to about 3 μg. In some embodiments, the topical steroid comprises difluprednate 0.05% at a dose of about 2.5 μg.

In some aspects, the invention provides a unit dose of about 6×10¹¹ vector genomes (vg) or less of recombinant adeno-associated virus (rAAV) particles for use in a method for treating glaucoma in an individual, the method comprising administering said unit dose to one eye of the individual, wherein the individual is a human, and wherein the rAAV particles comprise: a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. In some aspects, the invention provides a unit dose of rAAV particles for use in a method for reducing intraocular pressure in an eye of an individual in need thereof, the method comprising administering said unit dose to one eye of the individual, wherein the individual is a human, and wherein the rAAV particles comprise: a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. In some embodiments, the individual has glaucoma. In some embodiments, the glaucoma is neovascular glaucoma.

### INCORPORATION BY REFERENCE

All references cited herein, including patent applications and publications, are incorporated by reference in their entirety.

### BRIEF DESCRIPTION OF THE DRAWING

FIG. 1 provides the nucleic acid sequence of aflibercept (SEQ ID NO: 36).

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Definitions

Unless otherwise defined, all technical terms used herein have the same meaning as commonly understood by one of ordinary skill in the art.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value. Reference to “about” a value or parameter herein includes (and describes) embodiments that are directed to that value or parameter per se.

The term “subject”, “patient”, or “individual” refers to primates, such as humans and non-human primates, e.g., African green monkeys and rhesus monkeys. In some embodiments, the subject is a human.

The terms “treat,” “treating”, “treatment,” “ameliorate” or “ameliorating” and other grammatical equivalents as used herein, refer to alleviating, abating or ameliorating an ocular disease or disorder, or symptoms of the ocular disease or disorder, preventing additional symptoms of the ocular disease or disorder, ameliorating or preventing the underlying metabolic causes of symptoms, inhibiting the ocular disease or disorder, e.g., arresting the development of the ocular disease or disorder, relieving the ocular disease or disorder, causing regression of the ocular disease or disorder, or stopping the symptoms of the ocular disease or disorder, and are intended to include prophylaxis. The terms further include achieving a therapeutic benefit and/or a prophylactic benefit. The term “therapeutic benefit” refers to eradication or amelioration of the ocular disease or disorder being treated. Also, a therapeutic benefit is achieved with the eradication or amelioration of one or more of the physiological symptoms associated with the ocular disease or disorder such that an improvement is observed in the subject, notwithstanding that, in some embodiments, the subject is still afflicted with the ocular disease or disorder. For prophylactic benefit, the pharmaceutical compositions are administered to a subject at risk of developing the ocular disease or disorder, or to a subject reporting one or more of the physiological symptoms of the ocular disease or disorder, even if a diagnosis of the disease or disorder has not been made.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The terms “effective amount”, “therapeutically effective amount” or “pharmaceutically effective amount” as used herein, can refer to a sufficient amount of at least one pharmaceutical composition or compound being administered which will relieve to some extent one or more of the symptoms of the ocular disease or disorder being treated. An “effective amount”, “therapeutically effective amount” or “pharmaceutically effective amount” of a pharmaceutical composition may be administered to a subject in need thereof as a unit dose (as described in further detail elsewhere herein).

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., aflibercept) for transduction into a target cell or to a target tissue. In general, the heterologous polynucleotide is flanked by at least one, and generally by two, AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” refers to a viral particle comprising at least one AAV capsid protein and a polynucleotide rAAV vector. In some cases, the at least one AAV capsid protein is from a wild type AAV or is a variant AAV capsid protein (e.g., an AAV capsid protein with an insertion, e.g., an insertion of the 7m8 amino sequence as set forth below). If the particle comprises a heterologous polynucleotide (e.g., a polynucleotide other than a wild-type AAV genome such as a transgene to be delivered to a target cell or target tissue), it is referred to as a “rAAV particle”, “rAAV vector particle” or a “rAAV vector”. Thus, production of rAAV particles necessarily includes production of a rAAV vector, as such a vector contained within a rAAV particle.

The term “packaging” as used herein can refer to a series of intracellular events that can result in the assembly and encapsidation of a rAAV particle.

AAV “rep” and “cap” genes refer to polynucleotide sequences encoding replication and encapsidation proteins of adeno-associated virus. AAV rep and cap are referred to herein as AAV “packaging genes.”

The term “polypeptide” can encompass both naturally occurring and non-naturally occurring proteins (e.g., a fusion protein), peptides, fragments, mutants, derivatives and analogs thereof. A polypeptide may be monomeric, dimeric, trimeric, or polymeric. Further, a polypeptide may comprise a number of different domains, each of which has one or more distinct activities. For the avoidance of doubt, a “polypeptide” may be any length greater two amino acids.

As used herein, “polypeptide variant” or simply “variant” refers to a polypeptide whose sequence contains an amino acid modification. In some embodiments, the modification is an insertion, duplication, deletion, rearrangement or substitution of one or more amino acids compared to the amino acid sequence of a reference protein or polypeptide, such as a native or wild type protein. A variant may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A variant can have the same or a different biological activity compared to the reference protein, or the unmodified protein.

In some embodiments, a variant can have, for example, at least about 80%, 85%, 90%, 91%, 92%, 93%, 94%, 95%, 96%, 97%, 98%, or 99% overall sequence homology to its counterpart reference protein. In some embodiments, a variant can have at least about 90% overall sequence homology to the wild-type protein. In some embodiments, a variant exhibits at least about 95%, at least about 98%, at least about 99%, at least about 99.5%, or at least about 99.9% overall sequence identity.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

The term “anti-VEGF agent” includes any therapeutic agent, including proteins, polypeptides, peptides, fusion protein, multimeric proteins, gene products, antibody, human monoclonal antibody, antibody fragment, aptamer, small molecule, kinase inhibitor, receptor or receptor fragment, or nucleic acid molecule, that can reduce, interfere with, disrupt, block and/or inhibit the activity or function of an endogenous VEGF and/or an endogenous VEGF receptor (VEGFR), or the VEGF-VEGFR interaction or pathway in vivo. An anti-VEGF agent can be any one of the known therapeutic agents that can reduce new blood vessel growth or formation and/or oedem, or swelling, when delivered into a cell, tissue, or a subject in vivo, e.g., ranibizumab, brolucizumab, or bevacizumab. In some embodiments, an anti-VEGF agent can be naturally occurring, non-naturally occurring, or synthetic. In some embodiments, an anti-VEGF agent can be derived from a naturally occurring molecule that was subsequently modified or mutated to confer an anti-VEGF activity. In some embodiments, an anti-VEGF agent is a fusion or chimeric protein. In such proteins, functional domains or polypeptides are artificially fused to a moiety or a polypeptide to make a fusion or chimeric protein that can sequester VEGF in vivo or function as a VEGFR decoy. In some embodiments, an anti-VEGF agent is a fusion or chimeric protein that blocks endogenous VEGFR from interacting with its ligands.

As used herein, “VEGF” can refer to any isoform of VEGF, unless required otherwise, including, but not limited to, VEGF-A, VEGF-B, VEGF-C, VEGF-D, VEGF-E, VEGF-F, or any combination, or any functional fragment or variant thereof. Unless required otherwise, “VEGF” can refer to any member of the VEGF family, including members: VEGF-A, placenta growth factor (PGF), VEGF-B, VEGF-C, and VEGF-D, or any combination, functional fragment, or variant thereof. As used herein, “VEGF receptor” or “VEGFR” or “VEGF-R” can be used to refer to any one of the receptors of VEGF, including, but not limited to, VEGFR-1 (or Flt-1), VEGFR-2 (or Flk-1/KDR), and VEGFR-3 (or Flt-4). VEGFR can be a membrane bound or soluble form, or a functional fragment or truncation of a receptor. Examples of anti-VEGF agents include, but are not limited to, ranibizumab, bevacizumab, brolucizumab, or any combination, variant, or functional fragment thereof.

“Operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

The term “expression vector” or “expression construct” or “cassette” or “plasmid” or simply “vector” can include any type of genetic construct, including AAV or rAAV vectors, containing a nucleic acid or polynucleotide coding for a gene product in which part or all of the nucleic acid encoding sequence is capable of being transcribed and is adapted for gene therapy. The transcript can be translated into a protein. In some embodiments, the transcript is partially translated or not translated. In certain aspects, expression includes both transcription of a gene and translation of mRNA into a gene product. In other aspects, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette,” a large number of which are known and available in the art or can be readily constructed from components that are available in the art.

The term “heterologous” can refer to an entity that is genotypically distinct from that of the rest of the entity to which it is being compared. For example, a polynucleotide introduced by genetic engineering techniques into a plasmid or vector derived from a different species can be a heterologous polynucleotide. A promoter removed from its native coding sequence and operatively linked to a coding sequence with which it is not naturally found linked can be a heterologous promoter.

As used herein, “7m8” refers to the amino acid sequence LALGETTRPA (SEQ ID NO: 1).

“7m8 variant” refers to a rAAV, which can be of any serotype, with the amino acid sequence LALGETTRPA (SEQ ID NO: 1) inserted in the solvent exposed GH loop of the capsid protein.

When 7m8 is inserted in a rAAV2 (also referred to as AAV2.7m8), the amino acid sequence LALGETTRPA (SEQ ID NO: 1) is inserted into the GH loop within amino acids 570-611 of the AAV2 capsid protein, e.g., between positions 587 and 588 of the AAV2 capsid protein, VP1. In some cases, when 7m8 is inserted in a rAAV2 (also referred to as AAV2.7m8), the amino acid sequence LALGETTRPA (SEQ ID NO: 1) is inserted into the GH loop of the AAV2 capsid protein, e.g., between positions 587 and 588 of AAV2 VP1 comprising the sequence of SEQ ID NO: 13. When 7m8 is inserted in a rAAV1 (also referred to as AAV1.7m8), the amino acid sequence LALGETTRPA (SEQ ID NO: 1) is inserted into the GH loop within amino acids 571-612 of the AAV1 capsid protein, e.g., between amino acids 590 and 591 of the AAV1 capsid protein. When 7m8 is inserted in a rAAV5 (also referred to as AAV5.7m8), the amino acid sequence LALGETTRPA (SEQ ID NO: 1) is inserted into the GH loop withii amino acids 560-601 of the AAV5 capsid protein, e.g., between amino acids 575 and 576 of the AAV5 capsid protein. When 7m8 is inserted in a rAAV6 (also referred to as AAV6.7m8), the amino acid sequence LALGETTRPA (SEQ ID NO: 1) is inserted into the GH loop within amino acids 571 to 612 of the AAV6 capsid protein, e.g., between amino acids 590 and 591 of the AAV6 capsid protein. When 7m8 is inserted in a rAAV7 (also referred to as AAV7.7m8), the amino acid sequence LALGETTRPA (SEQ ID NO: 1) is inserted into the GH loop within amino acids 572 to 613 of the AAV7 capsid protein, e.g., between amino acids 589 and 590 of the AAV7 capsid protein. When 7m8 is inserted in a rAAV8 (also referred to as AAV8.7m8), the amino acid sequence LALGETTRPA (SEQ ID NO: 1) is inserted into the GH loop withii amino acids 573 to 614 of the AAV8 capsid protein, e.g., between amino acids 590 and 591 of the AAV8 capsid protein. When 7m8 is inserted in a rAAV9 (also referred to as AAV9.7m8), the amino acid sequence LALGETTRPA (SEQ ID NO: 1) is inserted into the GH loop of the AAV9 capsid protein, e.g., between amino acids 588 and 589 of the AAV9 capsid protein. When 7m8 is inserted in a rAAV10 (also referred to as AAV10.7m8), the amino acid sequence LALGETTRPA (SEQ ID NO: 1) is inserted into the GH loop within amino acids 573 to 614 of the AAV10 capsid protein, e.g., between amino acids 589 and 590 of the AAV10 capsid protein.

### Overview

Current therapies (e.g., aflibercept recombinant protein, ranibizumab recombinant protein) for ocular diseases require lifelong treatment, such as IVT administration approximately every 4-8 weeks. This can increase the risk of inflammation, infection, and other adverse effects in some patients. Further, current therapies create compliance challenges due to repeated and/or frequent trips to medical offices for administration of the therapy. Reduction in frequency of administration is associated with vision loss and deterioration of the eye disease or condition. The ability of AAV vectors to efficiently transduce target retinal cells following IVT injection has been exploited to successfully transfer therapeutic genes into photoreceptors, retinal pigment epithelium, and the inner retina to treat a variety of retinal diseases. Thus, administration of rAAV particles encoding an anti-VEGF agent (e.g., aflibercept) can provide prolonged and/or sustained release of the anti-VEGF agent in vivo.

Accordingly, the present disclosure provides methods of treating an ocular disease (e.g., glaucoma) in an individual by administering a single unit dose of 6×10¹¹ vg/eye or less of rAAV particles encoding an anti-VEGF agent (e.g., aflibercept). In addition, the present disclosure provides methods for reducing intraocular pressure (IOP) in the eye of an individual with an ocular disease (e.g., glaucoma) by administering a single unit dose of rAAV particles encoding an anti-VEGF agent (e.g., aflibercept). The methods disclosed herein reduce or eliminate the need for repeated IVT injections while providing long-term efficacy, thereby addressing the non-compliance and non-adherence problem. In addition, the methods provided herein reduce the adverse effects associated with multiple IVT injections.

### Methods of Treatment

Provided herein is a method for treating an ocular disease (e.g., glaucoma) in an individual, the method comprising administering a unit dose of recombinant adeno-associated virus (rAAV) particles to an eye of the individual.

Also provided herein is a method for reducing intraocular pressure an individual (e.g., in the eye of an individual with an ocular disease (e.g., glaucoma)), the method comprising administering a unit dose of rAAV particles to an eye of the individual.

Also provided herein is a method for treating an ocular disease (e.g., glaucoma) in an individual, the method comprising administering an anti-VEGF agent (e.g., aflibercept) to an eye of the individual, and administering treatment (e.g., at least one, at least two, a unit dose of recombinant adeno-associated virus (rAAV) particles to the eye of the individual after administration of the anti-VEGF agent.

In some embodiments, the ocular disease is glaucoma. In some embodiments, the glaucoma is neovascular glaucoma.

In some embodiments, the individual is a human. In some embodiments, the individual received at least one prior at least three, at least four, at least 5 or more treatments) for the ocular disease. In some embodiments, the at least one prior treatment (e.g., at least one, at least two, at least three, at least four, at least 5 or more treatments) comprised an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept). In some embodiments, the individual received treatment (e.g., with any one or more of the agents described above) within about the last 8 weeks, about the last 9 weeks, about the last 10 weeks, about the last 11 weeks, about the last 12 weeks, about the last 13 weeks, about the last 14 weeks, about the last 15 weeks, or about the last 16 weeks prior to administration of the unit dose of rAAV particles. In some embodiments, the individual demonstrated a meaningful response to a prior treatment. In some embodiments, the individual demonstrated a meaningful response to a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept). In some embodiments, the anti-VEGF agent is aflibercept, a functional variant thereof, or a functional fragment thereof. In some embodiments, the anti-VEGF agent comprises a polypeptide comprising an amino acid sequence with at least about 95% identity to the amino acid sequence of SEQ ID NO: 35. In some embodiments, the anti-VEGF agent is aflibercept, a functional variant thereof, or a functional fragment thereof. In some embodiments, the anti-VEGF agent comprises a polypeptide comprising an amino acid sequence with at least about 95% identity to the amino acid sequence of SEQ ID NO: 35.

In some embodiments, some embodiments, the individual has vision impairment. In some embodiments, the individual has visual acuity (BCVA) of between about 78 to 50 ETDRS letters (e.g., any of 50, 51, 52, 53 54, 55, 56, 57, 58, 59, 60, 61, 62, 63, 64, 65, 66, 67, 68, 69, 70, 71, 72, 73, 74, 75, 76, 77, or 78 ETDRS letters) in the eye administered the rAAV particles prior to administration of the unit dose of rAAV particles. In some embodiments, the individual has visual acuity (Snellen equivalent) of between about 20/32 to about 20/100 in the eye administered the rAAV particles prior to administration of the unit dose of rAAV particles.

In some embodiments, the individual has a central subfield thickness (CST) of ≥325 m using Heidelberg Spectralis® with center-involving IRF (center 1 mm) in the eye administered the rAAV particles prior to administration of the unit dose of rAAV particles. In some embodiments, the individual has a decrease in vision in the eye administered the rAAV particles prior to administration of the unit dose of rAAV particles that is primarily due to glaucoma (e.g., neovascular glaucoma). In some embodiments, the individual was diagnosed with glaucoma (e.g., neovascular glaucoma) in the eye administered the rAAV particles about 6 months or less prior to administration of the unit dose of rAAV particles, e.g., any of about 6 months, about 5 months, about 4 months, about 3 months, about 2 months, about 1 month, or less, prior to administration of the unit dose of rAAV particles. In some embodiments, the individual received the prior treatments with an anti-VEGF agent in the eye administered the rAAV particles at least about 60 days (i.e., about 2 months) prior to administration of the unit dose of rAAV particles. In some embodiments, the individual exhibited a meaningful response in central subfield thickness to the prior treatments with an anti-VEGF agent in the eye administered the rAAV particles prior to administration of the unit dose of rAAV particles, for example, at least a 10% reduction in central subfield thickness. In some embodiments, the individual did not experience an adverse reaction to the prior treatments with an anti-VEGF agent prior to administration of the unit dose of rAAV particles. In some embodiments, the individual does not have neutralizing antibodies to AAV2.7m8 prior to administration of the unit dose of rAAV particles. In some embodiments, the individual does not have an anti-AAV2.7m8 neutralizing antibody titer of greater than 1:125 prior to administration of the unit dose of rAAV particles, e.g., within about 6 months prior to administration of the unit dose of rAAV particles.

In some embodiments, the individual does not have a history of allergy to aflibercept, corticosteroids, or fluorescein dye or sodium fluorescein (e.g., used in angiography) prior to administration of the unit dose of rAAV particles. In some embodiments, the individual has a history of mild allergy to aflibercept, corticosteroid, or fluorescein dye or sodium fluorescein (e.g., used in angiography) prior to administration of the unit dose of rAAV particles, wherein the allergy is amenable to treatment.

In some embodiments, the individual does not have uncontrolled diabetes, e.g., HbA1C of greater than 10%, prior to administration of the unit dose of rAAV particles. In some embodiments, the individual does not have a history of diabetic ketoacidosis within about 3 months prior to administration of the unit dose of rAAV particles. In some embodiments, the individual has not initiated intensive insulin treatment, e.g., with an insulin pump or multiple daily insulin injections, prior to administration of the unit dose of rAAV particles.

In some embodiments, the individual does not plan to initiate intensive insulin treatment, e.g., with an insulin pump or multiple daily insulin injections, within about 3 months after administration of the unit dose of rAAV particles. In some embodiments, the individual does not have a history of systemic autoimmune disease that requires treatment with systemic steroids or immunosuppressive treatments, e.g., methotrexate or adalimumab, prior to administration of the unit dose of rAAV particles. In some embodiments, the individual is not being administered a systemic drug known to cause macular edema, such as fingolimod, tamoxifen, chloroquine, or hydroxychloroquine, prior to administration of the unit dose of rAAV particles. In some embodiments, the individual is not being administered a systemic anti-VEGF treatment prior to administration of the unit dose of rAAV particles. In some embodiments, the individual does not have high-risk proliferative diabetic retinopathy (PDR) in the eye administered the rAAV particles prior to administration of the unit dose of rAAV particles. In some embodiments, PDR is defined as any vitreous or preretinal hemorrhage, neovascularization elsewhere >½-disc area within an area equivalent to standard ETDRS 7-field on clinical examination, or neovascularization of disc >⅓-disc area on clinical examination. In some embodiments, the individual does not have focal or grid laser photocoagulation in the eye administered the rAAV particles prior to administration of the unit dose of rAAV particles. In some embodiments, the individual does not have any prior pan retinal photocoagulation (PRP) in the eye administered the rAAV particles prior to administration of the unit dose of rAAV particles. In some embodiments, the individual has not received an anti-VEGF therapy (e.g., aflibercept IVT injections) in the eye administered the rAAV particles prior to administration of the unit dose of rAAV particles. In some embodiments, the individual has not received an anti-VEGF therapy (e.g., aflibercept IVT injections) in the eye administered the rAAV particles for at least 60 days prior to administration of the unit dose of rAAV particles. In some embodiments, the individual has not received more than two anti-VEGF treatments (e.g., aflibercept IVT injections) in the eye administered the rAAV particles prior to administration of the unit dose of rAAV particles.

In some embodiments, the individual does not have a history of any of anterior segment neovascularization (e.g., neovascularization of the iris [NVI] or neovascular glaucoma [NVG]), significant vitreous hemorrhage, fibrovascular proliferation, or tractional retinal detachment in the eye administered the rAAV particles prior to administration of the unit dose of rAAV particles. In some embodiments, the individual does not have structural abnormalities at the fovea (e.g., any of dense hard exudates, pigment abnormalities, foveal atrophy, vitreomacular traction or epiretinal membrane) in the eye administered the rAAV particles that contribute to macular edema or visual impairment prior to administration of the unit dose of rAAV particles. In some embodiments, structural abnormalities at the fovea are assessed on clinical examination or OCT. In some embodiments, the individual does not have a history of retinal disease other than diabetic retinopathy (e.g., age-related macular degeneration (in either eye), retinal vein occlusion, retinal arterial occlusion, or pathologic myopia) in the eye administered the rAAV particles prior to administration of the unit dose of rAAV particles. In some embodiments, the individual does not have history of ocular disease other than diabetic macular edema in the eye administered the rAAV particles, e.g., a significant cataract or macular traction, or evidence of posterior subcapsular cataract, prior to administration of the unit dose of rAAV particles. In some embodiments, the individual does not have history of cataract extraction or Yttrium Aluminum Garnet (YAG) capsulotomy in the eye administered the rAAV particles within at least about 3 months prior to administration of the unit dose of rAAV particles. In some embodiments, the individual does not have a history of retinal detachment (with or without repair) in the eye administered the rAAV particles prior to administration of the unit dose of rAAV particles. In some embodiments, the individual does not have a history of any of trabeculectomy, glaucoma shunt, or minimally invasive glaucoma surgery (MIGS) in the eye administered the rAAV particles prior to administration of the unit dose of rAAV particles. In some embodiments, the individual does not have a history of vitrectomy or other filtration surgery in the eye administered the rAAV particles prior to administration of the unit dose of rAAV particles. In some embodiments, the individual does not have aphakia or presence of an anterior chamber intraocular lens in the eye administered the rAAV particles prior to administration of the unit dose of rAAV particles. In some embodiments, the individual does not have uncontrolled ocular hypertension or glaucoma in the eye administered the rAAV particles, e.g., IOP>22 mmHg despite treatment with anti-glaucoma medication or current use of >2 IOP lowering medications, prior to administration of the unit dose of rAAV particles. In some embodiments, the individual does not have a history of intraocular or periocular steroid treatment for any ocular condition (e.g., IVT Triesence, Iluvien or Ozurdex) in the eye administered the rAAV particles prior to administration of the unit dose of rAAV particles. In some embodiments, the individual has not had refractive surgery in the eye administered the rAAV particles within at least about 90 days prior to administration of the unit dose of rAAV particles. In some embodiments, the individual does not have previous penetrating keratoplasty, endothelial keratoplasty, or ocular radiation in the eye administered the rAAV particles prior to administration of the unit dose of rAAV particles. In some embodiments, the individual has not had any prior vitreoretinal surgery in the eye administered the rAAV particles prior to administration of the unit dose of rAAV particles. In some embodiments, the individual does not have a history of uveitis or intraocular inflammation, e.g., grade trace or above except mild anticipated post-operative inflammation that resolved, prior to administration of the unit dose of rAAV particles. In some embodiments, the individual does not have a history of IOP elevation that is related to topical steroid administration prior to administration of the unit dose of rAAV particles. In some embodiments, the individual does not have a history of ocular Herpes Simplex Virus (HSV), Varicella-zoster virus (VZV), or Cytomegalovirus (CMV), including viral uveitis, retinitis or keratitis prior to administration of the unit dose of rAAV particles. In some embodiments, the individual does not have evidence of any of external ocular infection, including conjunctivitis, chalazion, or significant blepharitis prior to administration of the unit dose of rAAV particles. In some embodiments, the individual does not have history of ocular toxoplasmosis prior to administration of the unit dose of rAAV particles.

In some embodiments, the unit dose is expressed as the number of vector genomes (vg). In some embodiments, the unit dose is about 6×10¹¹ vector genomes (vg) or less of the rAAV particles. In some embodiments, the unit dose is expressed as the number of vector genomes (vg) per eye (vg/eye). In some embodiments, the unit dose is about 6×10¹¹ vg/eye or less of the rAAV particles. In some embodiments, the unit dose of rAAV particles is about 6×10¹⁰ to about 2×10¹¹ vg/eye. In some embodiments, the unit dose of rAAV particles is about 2×10¹¹ or about 6×10¹⁰ vg/eye.

In some embodiments, the unit dose of rAAV particles is administered to one eye of the individual. In some embodiments, the one eye of the individual is the right eye or the left eye. In some embodiments, the one eye of the individual is the right eye. In some embodiments, the one eye of the individual is the left eye. In some embodiments, the methods provided herein further comprise administering a unit dose of rAAV particles to the contralateral eye of the individual. In some embodiments, the one eye of the individual is the right eye and the contralateral eye is the left eye. In some embodiments, the one eye of the individual is the left eye and the contralateral eye is the right eye.

In some embodiments, the administering the unit dose of rAAV particles to the contralateral eye of the individual is up to about 2 weeks (e.g., about 0 days, 1 day, 2 days, 3 days, 4 days, 5 days, 6 days, 7 days, 8 days, 9 days, 10 days, 11 days, 12 days, 13 days, or 14 days) after administering the unit dose of rAAV particles to the one eye. In some embodiments, the unit dose of rAAV particles administered to the contralateral eye of the individual is about the same as (e.g., less than 1% higher or lower, less than 5% higher or lower, less than 10% higher or lower, or less than 20% higher or lower) or lower (e.g., about 5%, about 10%, about 20%, about 30%, about 40%, about 50%, about 60%, about 70%, about 80%, or about 90% lower) than the unit dose of rAAV particles administered to the one eye of the individual.

In some embodiments, the administering the unit dose of rAAV particles to the contralateral eye is at least about 2 weeks (e.g., at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, or more) after administering the unit dose of rAAV particles to the one eye. In some embodiments, the unit dose of rAAV particles administered to the contralateral eye of the individual is higher (e.g., any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more, higher) than the unit dose of rAAV particles administered to the one eye of the individual.

In some embodiments, the rAAV particles comprise a) a nucleic acid encoding a polypeptide comprising an amino acid sequence with at least about 95%, at least about 96%, at least about 97%, at least about 98%, at least about 99%, at least about 99.99%, or 100% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. The sequence of SEQ ID NO: 35 is provided below:

(SEQ ID NO: 35)
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPG

In some embodiments, the rAAV particles comprise a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

In some embodiments, the rAAV particles comprise a nucleic acid encoding a polypeptide comprising an amino acid sequence with at least about 80%, at least about 85%, at least about 90%, at least about 95%, at least about 96%, at least about 97%, at least about 98%, at least about 99%, at least about 99.99%, or 100% identity to the amino acid sequence of SEQ ID NO: 35 and flanked by AAV2 inverted terminal repeats (ITRs). In some embodiments, the rAAV particles comprise a nucleic acid encoding a polypeptide comprising an amino acid sequence with at least about 95% identity to the amino acid sequence of SEQ ID NO: 35 and flanked by AAV2 inverted terminal repeats (ITRs). In some embodiments, the polypeptide comprises the amino acid sequence of SEQ ID NO: 35. In some embodiments, the polypeptide is aflibercept or a functional variant thereof or functional fragment thereof.

In some embodiments, the rAAV particles comprise a nucleic acid comprising a codon-optimized sequence encoding an amino acid sequence with at least about 80%, at least about 85%, at least about 90%, at least about 95%, at least about 96%, at least about 97%, at least about 98%, at least about 99%, at least about 99.99%, or 100% identity to the amino acid sequence of SEQ ID NO: 35 and flanked by AAV2 inverted terminal repeats (ITRs). In some embodiments, the rAAV particles comprise a nucleic acid comprising a codon-optimized sequence encoding an amino acid sequence with at least about 95% identity to the amino acid sequence of SEQ ID NO: 35 and flanked by AAV2 inverted terminal repeats (ITRs). In some embodiments, the rAAV particles comprise a nucleic acid comprising a codon-optimized sequence encoding an amino acid sequence with 100% identity to the amino acid sequence of SEQ ID NO: 35 and flanked by AAV2 inverted terminal repeats (ITRs).

In some embodiments, the rAAV particles comprise a nucleic acid comprising the cDNA sequence of aflibercept or a functional variant thereof or functional fragment thereof and flanked by AAV2 inverted terminal repeats (ITRs). In some embodiments, the rAAV particles comprise a nucleic acid comprising a codon-optimized cDNA sequence of aflibercept or a functional variant thereof or functional fragment thereof and flanked by AAV2 inverted terminal repeats (ITRs). In some embodiments, the rAAV particles comprise a nucleic acid comprising the nucleic acid sequence of SEQ ID NO: 36.

In some embodiments, the nucleic acid further comprises (a) a first enhancer region comprising a CMV sequence; (b) a promoter region comprising a CMV sequence; (c) a 5′UTR region comprising, in the 5′ to 3′ order, a TPL sequence and an eMLP sequence; (d) a second enhancer region comprising a full EES sequence; and (e) a HGH polyadenylation site. In some embodiments, the enhancer region comprising a CMV sequence comprises the sequence of SEQ ID NO: 22. In some embodiments, the promoter region comprising a CMV sequence comprises the sequence of SEQ ID NO: 23. In some embodiments, the TPL sequence comprises the sequence of SEQ ID NO: 24. In some embodiments, the eMLP sequence comprises the sequence of SEQ ID NO: 25. In some embodiments, the second enhancer region comprising a full EES sequence comprises the sequence of SEQ ID NO: 26. In some embodiments, the HGH polyadenylation site comprises the sequence of SEQ ID NO: 27.

In some embodiments, the rAAV particles comprise an AAV2 capsid protein comprising the amino acid sequence LGETTRP (SEQ ID NO: 14) inserted between positions 587 and 588 of the AAV2 VP1 comprising the sequence of SEQ ID NO: 13. The sequence of SEQ ID NO: 13 is provided below:

(SEQ ID NO: 13)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNR
QAATADVNTQGVLPGMVWQDRDVYLQGPIWAKIPHTDGHFHPSPLMGGF
GLKHPPPQILIKNTPVPANPSTTFSAAKFASFITQYSTGQVSVEIEWEL
QKENSKRWNPEIQYTSNYNKSVNVDFTVDINGVYSEPRPIGTRYLTRNL

In some embodiments, the rAAV particles comprise an AAV2 capsid protein comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise an AAV2 capsid protein comprising the amino acid sequence LALGETTRPA (SEQ ID NO: 1) inserted between positions 587 and 588 of the AAV2 VP1 comprising the sequence of SEQ ID NO: 13.

In some embodiments, the rAAV particles comprise an AAV2 capsid protein comprising any of the following amino acid sequences inserted between positions 587 and 588 of the capsid protein, wherein the amino acid residue numbering corresponds to an AAV2 VP1 capsid protein: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21). In some embodiments, the rAAV particles comprise an AAV2 capsid protein comprising any of the following amino acid sequences inserted between positions 587 and 588 of the AAV2 VP1 comprising the sequence of SEQ ID NO: 13: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21).

In some embodiments, the administration of the unit dose of rAAV particles to the one eye and/or to the contralateral eye of the individual is by intravitreal (IVT) injection, intraocular administration, or intraretinal injection. In some embodiments, the administration of the unit dose of rAAV particles to the one eye and/or to the contralateral eye of the individual is by intravitreal (IVT) injection.

In some embodiments, the unit dose of rAAV particles is in a pharmaceutical formulation. In some embodiments, the pharmaceutical formulation comprises the rAAV particles, one or more osmotic or ionic strength agents, one or more buffering agents, one or more surfactants, and one or more solvents. In some embodiments, the osmotic or ionic strength agent is sodium chloride. In some embodiments, the one or more buffering agents are sodium phosphate monobasic and/or sodium phosphate dibasic. In some embodiments, the surfactant is Poloxamer 188. In some embodiments, the solvent is water. In some embodiments, the pharmaceutical formulation comprises the rAAV particles, sodium chloride, sodium phosphate and a surfactant. In some embodiments, the pharmaceutical formulation comprises about 1×10¹⁰ vg/mL to about 1×10¹³ vg/mL of rAAV particles. In some embodiments, the pharmaceutical formulation comprises about 6×10¹¹ vg/mL to about 6×10¹² vg/mL of rAAV particles. In some embodiments, the pharmaceutical formulation comprises about 150 mM to about 200 mM sodium chloride (e.g., any of about 150 mM, about 160 mM, about 170 mM, about 180 mM, about 190 mM, or about 200 mM). In some embodiments, the pharmaceutical formulation comprises about 1 mM to about 10 mM monobasic sodium phosphate (e.g., about 1 mM, about 2 mM, about 3 mM, about 4 mM, about 5 mM, about 6 mM, about 7 mM, about 8 mM, about 9 mM, or about 10 mM). In some embodiments, the pharmaceutical formulation comprises about 1 mM to about 10 mM dibasic sodium phosphate (e.g., about 1 mM, about 2 mM, about 3 mM, about 4 mM, about 5 mM, about 6 mM, about 7 mM, about 8 mM, about 9 mM, or about 10 mM). In some embodiments, the pharmaceutical formulation comprises about 0.0005% (w/v) to about 0.005% (w/v) poloxamer 188 (e.g., any of about 0.0005% (w/v), 0.0006% (w/v), 0.0007% (w/v), 0.0008% (w/v), 0.0009% (w/v), 0.001% (w/v), 0.002% (w/v), 0.003% (w/v), 0.004% (w/v), or about 0.005% (w/v)). In some embodiments, the pharmaceutical formulation has a pH of about 7.0 to about 7.5 (e.g., any of about 7.0, about 7.1, about 7.2, about 7.3, about 7.4, or about 7.5). In some embodiments, the pharmaceutical formulation comprises about 6×10¹² vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3. In some embodiments, the pharmaceutical formulation comprises about 6×10¹¹ vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3.

In some embodiments, the unit dose of rAAV particles comprises a volume of between about 25 μL to about 250 μL (e.g., any of about 25 μL, about 30 μL, about 40 μL, about 50 μL, about 60 μL, about 70 μL, about 80 μL, about 90 μL, about 100 μL, about 10 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, or about 250 μL). In some embodiments, the concentration of rAAV particles in the pharmaceutical formulation is adjusted such that the volume of the unit dose of rAAV particles administered to an eye of the individual is between about 25 μL to about 250 μL. In some embodiments, the unit dose of rAAV particles comprises a volume of about 100 μL. In some embodiments, the unit dose of rAAV particles comprises a volume of about 30 μL.

In some embodiments, the unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is an ophthalmic steroid treatment. In some embodiments, the ophthalmic steroid treatment is a topical steroid treatment (e.g., a drop), a periocular steroid treatment (e.g., subtenons, subconjunctival), an intravitreal steroid treatment, or a superchoroidal steroid treatment. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. In some embodiments, the topical steroid treatment is a difluprednate treatment. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment (e.g., difluprednate). In some embodiments, the ophthalmic steroid treatment (e.g., difluprednate) is a daily steroid treatment for up to about 4 weeks, about 6 weeks, or about 8 weeks from administering the unit dose of rAAV particles. In some embodiments, the ophthalmic steroid treatment comprises about four administrations of ophthalmic steroid on about week 1, about three administrations of ophthalmic steroid on about week 2, about two administrations of ophthalmic steroid on about week 3, and about one administration of ophthalmic steroid on about week 4; timing starting with and following administration of the unit dose of rAAV particles. In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the steroid treatment is an ophthalmic steroid treatment (e.g., difluprednate). In some embodiments, the ophthalmic steroid treatment (e.g., difluprednate) is a daily topical steroid treatment for up to about 4 weeks, about 6 weeks, or about 8 weeks from administering the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid (i.e., QID) per day for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the topical steroid comprises difluprednate 0.05% at a dose of about 1 μg to about 3 μg. In some embodiments, the topical steroid comprises difluprednate 0.05% at a dose of about 2.5 μg. In some embodiments, the topical steroid is about 0.005% to about 0.5% difluprednate. In some embodiments, the topical steroid is any of about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% difluprednate. In some embodiments, the topical steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the administering the unit dose of rAAV particles to the one eye and/or to the contralateral eye of the individual results in maintenance or a decrease in macular volume compared to the macular volume prior to administration of the unit dose of rAAV particles. In some embodiments, the administering the unit dose of rAAV particles to the one eye and/or to the contralateral eye of the individual results in a decrease in macular volume compared to the macular volume prior to administration of the unit dose of rAAV particles. In some embodiments, the administering the unit dose of rAAV particles to the one eye and/or to the contralateral eye of the individual results in a decrease in macular volume of more than any of about 5%, about 10%, about 15%, about 20%, about 25%, about 30%, about 35%, about 40%, about 45%, or about 50% compared to the macular volume prior to administration of the unit dose of rAAV particles. In some embodiments, the administering the unit dose of rAAV particles to the one eye and/or to the contralateral eye of the individual results in a decrease in macular volume of at least about 10% compared to the macular volume prior to administration of the unit dose of rAAV particles. In some embodiments, the macular volume is determined by OCT or SD-OCT. In some embodiments, the administering the unit dose of rAAV particles to the one eye and/or to the contralateral eye of the individual results in a decrease in macular volume of at least about 10% compared to the macular volume prior to administration of the unit dose of rAAV particles. In some embodiments, the administering the unit dose of rAAV particles to the one eye and/or to the contralateral eye of the individual results in a decrease in macular volume of about 15% or more compared to the macular volume prior to administration of the unit dose of rAAV particles. In some embodiments, the macular volume is determined by OCT or SD-OCT.

In some embodiments, the maintenance or the decrease in macular volume compared to the macular volume prior to administration of the unit dose of rAAV particles is present at about 30 weeks or more after administration of the unit dose of rAAV particles to the one eye and/or to the contralateral eye of the individual. In some embodiments, the maintenance or the decrease in macular volume compared to the macular volume prior to administration of the unit dose of rAAV particles is present at any of about 30 weeks, about 34 weeks, about 44 weeks, about 6 months, about 1 year, about 1.5 years, about 2 years, about 3 years, about 5 years, about 10 years, or more after administration of the unit dose of rAAV particles to the one eye and/or to the contralateral eye of the individual.

In some embodiments, the administering the unit dose of rAAV particles to the one eye and/or to the contralateral eye of the individual results in maintenance or an improvement of visual acuity compared to the visual acuity prior to administration of the unit dose of rAAV particles. In some embodiments, the administering the unit dose of rAAV particles to the one eye and/or to the contralateral eye of the individual results in an improvement of visual acuity compared to the visual acuity prior to administration of the unit dose of rAAV particles. In some embodiments, the administering the unit dose of rAAV particles to the one eye and/or to the contralateral eye of the individual results in an improvement of visual acuity of more than any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more, compared to the visual acuity prior to administration of the unit dose of rAAV particles. In some embodiments, visual acuity is best corrected visual acuity (BCVA). In some embodiments, the administering the unit dose of rAAV particles to the one eye and/or to the contralateral eye of the individual results in an improvement of BCVA compared to the BCVA prior to administration of the unit dose of rAAV particles. In some embodiments, BCVA is expressed as an ETDRS score, which corresponds to the number of letters correctly read (Vitale et al., (2016) JAMA Opthalmol 134(9):1041:1047).

In some embodiments, the administering the unit dose of rAAV particles to the one eye and/or to the contralateral eye of the individual results in an improvement of BCVA of at least 15 ETDRS letters (Vitale et al., (2016) JAMA Opthalmol 134(9):1041:1047) (e.g., at least about 15, at least about 20, at least about 30, at least about 40, at least about 50, at least about 60, or about 70 letters) compared to the BCVA prior to administration of the unit dose of rAAV particles. In some embodiments, the administering the unit dose of rAAV particles to the one eye and/or to the contralateral eye of the individual results in an improvement of BCVA of between about 1 to about 15 (e.g., any of about 1, about 2, about 3, about 4, about 5, about 6, about 7, about 8, about 9, about 10, about 11, about 12, about 13, about 14, or about 15) ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 5 ETDRS letters compared to the BCVA prior to administration of the unit dose of rAAV particles. In some embodiments, the administering the unit dose of rAAV particles to the one eye and/or to the contralateral eye of the individual results in an improvement of BCVA of any of about 1, about 2, about 3, about 4, about 5, about 6, or about 7 ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 3 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 4 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 5.1 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 6.4 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 6.8 ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 8.8 ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 2.3 ETDRS letters compared to the BCVA prior to administration of the unit dose of rAAV particles.

In some embodiments, the administering the unit dose of rAAV particles to the one eye and/or to the contralateral eye of the individual results in maintenance of BCVA, wherein the individual loses fewer than 15 ETDRS letters (Vitale et al., (2016) JAMA Opthalmol 134(9):1041:1047) (e.g., any of 15 or less, 14 or less, 13 or less, 12 or less, 11 or less, 10 or less, 9 or less, 8 or less, 7 or less, 6 or less, 5 or less, 4 or less, 3 or less, 2 or less, 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses any of about 1, about 2, about 3, about 4, about 5, about 6, about 7, about 8, or about 9 ETDRS letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses 0 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 1 letter compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2.7 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2.8 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2 letters or less compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 3.2 letters or less compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 15 to about 0 letters (e.g., any of about 15, about 14, about 13, about 12, about 11, about 10, about 9, about 8, about 7, about 6, about 5, about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 10 to about 0 letters (e.g., any of about 10, about 9, about 8, about 7, about 6, about 5, about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 5 to about 0 letters (e.g., any of about 5, about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 4 to about 0 letters (e.g., any of about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 3 to about 0 letters (e.g., any of about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 2 to about 0 letters (e.g., any of about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 1 to about 0 letters compared to the BCVA prior to administration of the unit dose of rAAV particles.

In some embodiments, the administering the unit dose of rAAV particles to the one eye and/or to the contralateral eye of the individual results in a change in BCVA of between about −20 to +7 or more (e.g., any of −20, −19, −18, −17, −16, −15, −14, −13, −12, −11, −10, −9, −8, −7, −6, −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in an increase in BCVA of about any of 16, 7 or 5 ETDRS letters compared to the BCVA prior to administration of the unit dose of rAAV particles. In some embodiments, the administering the unit dose of rAAV particles to the one eye and/or to the contralateral eye of the individual results in a decrease in BCVA of about any of 19, 14, 7, 6, 5, 4, 3, 2, or 1 ETDRS letters compared to the BCVA prior to administration of the unit dose of rAAV particles. In some embodiments, the administering the unit dose of rAAV particles to the one eye and/or to the contralateral eye of the individual results in a decrease in BCVA of about 4.8 or about 0.8 ETDRS letters compared to the BCVA prior to administration of the unit dose of rAAV particles. In some embodiments, the administering the unit dose of rAAV particles to the one eye and/or to the contralateral eye of the individual results in a decrease in BCVA of about 2 ETDRS letters or less compared to the BCVA prior to administration of the unit dose of rAAV particles. In some embodiments, the administering the unit dose of rAAV particles to the one eye and/or to the contralateral eye of the individual results in a decrease in BCVA of about 3.2 ETDRS letters or less compared to the BCVA prior to administration of the unit dose of rAAV particles.

In some embodiments, the administering the unit dose of rAAV particles to the one eye and/or to the contralateral eye of the individual results in a change in BCVA of between about −15 to +7 or more (e.g., any of −15, −14, −13, −12, −11, −10, −9, −8, −7, −6, −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −10 to +7 or more (e.g., any of −10, −9, −8, −7, −6, −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −5 to +7 or more (e.g., any of −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −4 to +7 or more (e.g., any of −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −3 to +7 or more (e.g., any of −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −2 to +7 or more (e.g., any of −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −1 to +7 or more (e.g., any of −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about 0 to +7 or more (e.g., any of 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +1 to +7 or more (e.g., any of +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +2 to +7 or more (e.g., any of +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +3 to +7 or more (e.g., any of +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +4 to +7 or more (e.g., any of +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +5 to +7 or more (e.g., any of +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of about +6 or about +7 ETDRS letters, compared to the BCVA prior to administration of the unit dose of rAAV particles.

In some embodiments, administration of the unit dose of rAAV particles to the one eye and/or to the contralateral eye of the individual results in transient inflammation (e.g., inflammation driven by aqueous cells and/or vitreous cells, aqueous flare, posterior synchiae, poor pupil dilation). In some embodiments, administration of the unit dose of rAAV particles to the one eye and/or to the contralateral eye of the individual results in inflammation (e.g., inflammation driven by aqueous cells and/or vitreous cells, aqueous flare, posterior synchiae, poor pupil dilation) that is improved after administration of oral and/or topical steroid treatment and/or mydryatics. In some embodiments, administration of the unit dose of rAAV particles to the one eye and/or to the contralateral eye of the individual results in inflammation (e.g., inflammation driven by aqueous cells and/or vitreous cells) that resolves after administration of oral and/or topical steroid treatment. Inflammation (e.g., inflammation driven by aqueous cells and/or vitreous cells, aqueous flare, posterior synchiae, poor pupil dilation) may be measured using any method known in the art, such as the slit lamp exam.

In some embodiments, the maintenance or the improvement of visual acuity (e.g., BCVA) compared to the visual acuity prior to administration of the unit dose of rAAV particles is present at any of about 1 day, about 1 week, about 2 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, about 104 weeks, about 108 weeks, or more, after administration of the unit dose of rAAV particles to the one eye and/or to the contralateral eye of the individual. In some embodiments, the maintenance or the improvement of visual acuity (e.g., BCVA) compared to the visual acuity prior to administration of the unit dose of rAAV particles is present at about 30 weeks or more after administration of the unit dose of rAAV particles to the one eye and/or to the contralateral eye of the individual. In some embodiments, the maintenance or the improvement of visual acuity (e.g., BCVA) compared to the visual acuity prior to administration of the unit dose of rAAV particles is present at any of about 30 weeks, about 34 weeks, about 44 weeks, about 6 months, about 1 year, about 1.5 years, about 2 years, about 3 years, about 5 years, about 10 years, or more, after administration of the unit dose of rAAV particles to the one eye and/or to the contralateral eye of the individual.

In some embodiments, treatment (such as progress of treatment) of an ocular disease in an individual after administration of the unit dose of rAAV particles in the one eye and/or the contralateral eye is assessed based on best corrected visual acuity (BCVA) in the one eye and/or the contralateral eye. In some embodiments, BCVA is expressed as an ETDRS score, which corresponds to the number of letters correctly read (Vitale et al., (2016) JAMA Opthalmol 134(9):1041:1047). In some embodiments, an individual is determined to have maintenance of vision and/or visual acuity if the individual loses fewer than 15 letters in an ETDRS score (e.g., any of 15 or less, 14 or less, 13 or less, 12 or less, 11 or less, 10 or less, 9 or less, 8 or less, 7 or less, 6 or less, 5 or less, 4 or less, 3 or less, 2 or less, 1, or 0 letters) compared to prior to administration of the unit dose of rAAV particles in the one eye and/or the contralateral eye. In some embodiments, an individual is determined to have an improvement of vision and/or visual acuity if the individual gains at least 15 letters (e.g., any of at least about 15, at least about 20, at least about 30, at least about 40, at least about 50, at least about 60, or about 70 letters) comparted to prior to administration of the unit dose of rAAV particles in the one eye and/or the contralateral eye.

In some embodiments, treatment (such as progress of treatment) of an ocular disease in an individual after administration of the unit dose of rAAV particles in the one eye and/or the contralateral eye is assessed based on macular volume in the one eye and/or the contralateral eye. In some embodiments, macular volume is determined by SD-OCT. In some embodiments, treatment (such as progress of treatment) of an ocular disease in an individual after administration of the unit dose of rAAV particles in the one eye and/or the contralateral eye is determined if the macular volume assessed by SD-OCT is decreased after administration of the unit dose of rAAV particles in the one eye and/or the contralateral eye compared to prior to administration of the unit dose of rAAV particles in the one eye and/or the contralateral eye. In some embodiments, treatment (such as progress of treatment) of an ocular disease in an individual after administration of the unit dose of rAAV particles in the one eye and/or the contralateral eye is determined if the macular volume assessed by SD-OCT is maintained after administration of the unit dose of rAAV particles in the one eye and/or the contralateral eye compared to prior to administration of the unit dose of rAAV particles in the one eye and/or the contralateral eye.

In some embodiments, treatment (such as progress of treatment) of an ocular disease in an individual after administration of the unit dose of rAAV particles in the one eye and/or the contralateral eye is assessed based on decrease in iris rubeosis in the one eye and/or the contralateral eye. In some embodiments, decrease in iris rubeosis is determined by fluorescein angiography (FA). In some embodiments, treatment (such as progress of treatment) of an ocular disease in an individual after administration of the unit dose of rAAV particles in the one eye and/or the contralateral eye is determined if iris rubeosis (e.g., as assessed by FA) is decreased after administration of the unit dose of rAAV particles in the one eye and/or the contralateral eye compared to prior to administration of the unit dose of rAAV particles in the one eye and/or the contralateral eye. In some embodiments, treatment (such as progress of treatment) of an ocular disease in an individual after administration of the unit dose of rAAV particles in the one eye and/or the contralateral eye is determined if iris rubeosis (e.g., as assessed by FA) is maintained after administration of the unit dose of rAAV particles in the one eye and/or the contralateral eye compared to prior to administration of the unit dose of rAAV particles in the one eye and/or the contralateral eye.

In some embodiments, treatment (such as progress of treatment) of an ocular disease in an individual after administration of the unit dose of rAAV particles in the one eye and/or the contralateral eye is assessed based on decrease in intraocular pressure (IOP) in the one eye and/or the contralateral eye. In some embodiments, decrease in IOP is determined by Goldmann applanation tonometry test. In some embodiments, treatment (such as progress of treatment) of an ocular disease in an individual after administration of the unit dose of rAAV particles in the one eye and/or the contralateral eye is determined if IOP (e.g., determined by Goldmann applanation tonometry test) is decreased after administration of the unit dose of rAAV particles in the one eye and/or the contralateral eye compared to prior to administration of the unit dose of rAAV particles in the one eye and/or the contralateral eye. In some embodiments, treatment (such as progress of treatment) of an ocular disease in an individual after administration of the unit dose of rAAV particles in the one eye and/or the contralateral eye is determined if IOP (e.g., determined by Goldmann applanation tonometry test) is maintained after administration of the unit dose of rAAV particles in the one eye and/or the contralateral eye compared to prior to administration of the unit dose of rAAV particles in the one eye and/or the contralateral eye.

In some embodiments, treatment (such as progress of treatment) of an ocular disease in an individual after administration of the unit dose of rAAV particles in the one eye and/or the contralateral eye is assessed based on increase in the anterior chamber angle or anterior chamber depth in the one eye and/or the contralateral eye. In some embodiments, increase in the anterior chamber angle or anterior chamber depth is determined by gonioscopy, ultrasound biomicroscopy (UJBM), or anterior segment optical coherence tomography (OCT). In some embodiments, treatment (such as progress of treatment) of an ocular disease in an individual after administration of the unit dose of rAAV particles in the one eye and/or the contralateral eye is determined if the anterior chamber angle or anterior chamber depth (e.g., determined by gonioscopy, UBM, or OCT) is increased after administration of the unit dose of rAAV particles in the one eye and/or the contralateral eye compared to prior to administration of the unit dose of rAAV particles in the one eye and/or the contralateral eye. In some embodiments, treatment (such as progress of treatment) of an ocular disease in an individual after administration of the unit dose of rAAV particles in the one eye and/or the contralateral eye is determined if the anterior chamber angle or anterior chamber depth (e.g., determined by gonioscopy, UBM, or OCT) is maintained after administration of the unit dose of rAAV particles in the one eye and/or the contralateral eye compared to prior to administration of the unit dose of rAAV particles in the one eye and/or the contralateral eye.

In some embodiments, treatment (such as progress of treatment) of an ocular disease in an individual after administration of the unit dose of rAAV particles in the one eye and/or the contralateral eye is assessed based on the number of rescue therapy treatments (e.g., aflibercept injections) required by the individual after administration of the unit dose of rAAV particles in the one eye and/or the contralateral eye. In some embodiments, treatment (such as progress of treatment) of an ocular disease in an individual after administration of the unit dose of rAAV particles in the one eye and/or the contralateral eye is determined if an individual requires less than one rescue therapy treatment (e.g., aflibercept injection) any of every 4 weeks, every 5 weeks, every 6 weeks, every 7 weeks, every 8 weeks, every 9 weeks, every 10 weeks, or more, after administration of the unit dose of rAAV particles in the one eye and/or the contralateral eye.

In some embodiments, treatment (such as progress of treatment) of an ocular disease in an individual after administration of the unit dose of rAAV particles in the one eye and/or the contralateral eye is determined if an individual does not require any rescue therapy treatment (e.g., aflibercept injection) for any of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 15 weeks, at least 20 weeks, at least 30 weeks, at least 40 weeks, at least 50 weeks, at least 60 weeks, at least 70 weeks, at least 80 weeks, at least 90 weeks, at least 100 weeks, at least 110 weeks, or more, after administration of the unit dose of rAAV particles in the one eye and/or the contralateral eye.

In some embodiments, the individual does not require any rescue therapy treatment (e.g., aflibercept injection) for any of at least about 24 months, at least about 23 months, at least about 22 months, at least about 21 months, at least about 20 months, at least about 19 months, at least about 18 months, at least about 17 months, at least about 16 months, at least about 15 months, at least about 14 months, at least about 13 months, at least about 12 months, at least about 11 months, at least about 10 months, at least about 9 months, at least about 8 months, at least about 7 months, at least about 6 months, at least about 5 months, at least about 4 months, at least about 3 months, at least about 2 months, at least about 1 month, at least about 3 weeks, at least about 2 weeks, or at least about 1 week after administration of the unit dose of rAAV particles in the one eye and/or the contralateral eye. In some embodiments, the individual does not require any rescue therapy treatment (e.g., aflibercept injection) for at least about 12 months after administration of the unit dose of rAAV particles in the one eye and/or the contralateral eye. In some embodiments, the individual does not require any rescue therapy treatment (e.g., aflibercept injection) for at least about 10 months after administration of the unit dose of rAAV particles in the one eye and/or the contralateral eye. In some embodiments, the individual does not require any rescue therapy treatment (e.g., aflibercept injection) for at least about 7 months after administration of the unit dose of rAAV particles in the one eye and/or the contralateral eye. In some embodiments, the individual does not require any rescue therapy treatment (e.g., aflibercept injection) for at least about 6 months after administration of the unit dose of rAAV particles in the one eye and/or the contralateral eye. In some embodiments, the individual does not require any rescue therapy treatment (e.g., aflibercept injection) for at least about 2 months after administration of the unit dose of rAAV particles in the one eye and/or the contralateral eye. In some embodiments, the individual does not require any rescue therapy treatment (e.g., aflibercept injection) for at least about 1 month after administration of the unit dose of rAAV particles in the one eye and/or the contralateral eye.

In some embodiments, administration of a single unit dose of rAAV particles to the one eye and/or to the contralateral eye of a plurality of individuals results in at least about 50% (e.g., any of at least about 50%, at least about 55%, at least about 60%, at least about 65%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 67% (e.g., any of at least about 67%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 50% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 78% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 80% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 82% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection).

In some embodiments, administration of a single unit dose of rAAV particles to the one eye and/or to the contralateral eye of a plurality of individuals results in at least about 50% (e.g., any of at least about 50%, at least about 55%, at least about 60%, at least about 65%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection) for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more after administration of the rAAV particles. In some embodiments, administration of a single unit dose of rAAV particles to the one eye and/or to the contralateral eye of a plurality of individuals results in at least about 50% of the individuals in the plurality not requiring an anti-VEGF rescue treatment (e.g., aflibercept injection) for about 52 weeks or more, or about 56 weeks or more, after administration of the rAAV particles. In some embodiments, administration of a single unit dose of rAAV particles to the one eye and/or to the contralateral eye of a plurality of individuals results in at least about 67% (e.g., any of at least about 67%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a single unit dose of rAAV particles to the one eye and/or to the contralateral eye of a plurality of individuals results in at least about 78% of the individuals in the plurality not requiring an anti-VEGF rescue treatment (e.g., aflibercept injection) for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more, after administration of the rAAV particles. In some embodiments, administration of a single unit dose of rAAV particles to the one eye and/or to the contralateral eye of a plurality of individuals results in at least about 78% of the individuals in the plurality not requiring an anti-VEGF rescue treatment (e.g., aflibercept injection) for about 20 weeks or more, or about 36 weeks or more, after administration of the rAAV particles. In some embodiments, administration of a single unit dose of rAAV particles to the one eye and/or to the contralateral eye of a plurality of individuals results in at least about 80% of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a single unit dose of rAAV particles to the one eye and/or to the contralateral eye of a plurality of individuals results in at least about 82% of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection) for at least about 4 weeks after administration of the rAAV particles, e.g., any of any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more after administration of the rAAV particles. In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection) for any of about 64 weeks or more, 72 weeks or more, or 84 weeks or more, after administration of the rAAV particles.

In some embodiments, administration of a unit dose of rAAV particles in the one eye and/or the contralateral eye of a plurality of individuals results in about 78% or less (e.g., any of about 78% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2.5% or less, about 1% or less, or about 0.5% or less) of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in about 50% or less (e.g., any of about 50% or less, about 45% or less, about 40% or less, about 35% or less, about 30% or less, about 25% or less, about 20% or less, about 15% or less, about 10% or less, about 5% or less, about 2.5% or less, about 1% or less, or about 0.5% or less) of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in less than about 30% (e.g., less than any of about 30%, about 25%, about 20%, about 15%, about 10%, about 5%, about 2.5%, about 1%, or about 0.5%) of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in less than about 30% of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in less than about 20% of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in 0% of the individuals in the plurality requiring any rescue treatment (e.g., aflibercept injection) in the one eye and/or the contralateral eye.

In some embodiments, administration of a unit dose of rAAV particles in the one eye and/or the contralateral eye of a plurality of individuals results in about 78% or less (e.g., any of about 78% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2.5% or less, about 1% or less, or about 0.5% or less) of the individuals in the plurality requiring any rescue treatment (e.g., aflibercept injection) for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more, after administration of the rAAV particles. In some embodiments, administration of a unit dose of rAAV particles in the one eye and/or the contralateral eye of a plurality of individuals results in less than about 30% (e.g., less than any of about 30%, about 25%, about 20%, about 15%, about 10%, about 5%, about 2.5%, about 1%, or about 0.5%) of the individuals in the plurality requiring any rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a unit dose of rAAV particles in the one eye and/or the contralateral eye of a plurality of individuals results in less than about 30% of the individuals in the plurality requiring any rescue treatment (e.g., aflibercept injection) in the one eye and/or the contralateral eye for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a unit dose of rAAV particles in the one eye and/or the contralateral eye of a plurality of individuals results in less than about 20% of the individuals in the plurality requiring any rescue treatment (e.g., aflibercept injection) in the one eye and/or the contralateral eye for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a unit dose of rAAV particles in the one eye and/or the contralateral eye of a plurality of individuals results in 0% of the individuals in the plurality requiring any rescue treatment (e.g., aflibercept injection) in the one eye and/or the contralateral eye for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a unit dose of rAAV particles in the one eye and/or the contralateral eye of a plurality of individuals results in 0% of the individuals in the plurality requiring any rescue treatment (e.g., aflibercept injection) in the one eye and/or the contralateral eye for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more, after administration of the rAAV particles.

In some embodiments, administration of the unit dose of rAAV particles to the one eye and/or to the contralateral eye of a plurality of individuals results in a reduction in the mean annualized anti-VEGF injection rate of any of at least about 80%, at least about 85%, at least about 87%, at least about 90%, at least about 95%, at least about 99%, or 100%, compared to the mean annualized anti-VEGF injection rate prior to administration of the unit dose of rAAV particles. In some embodiments, administration of the unit dose of rAAV particles to the one eye and/or to the contralateral eye of a plurality of individuals results in a reduction in the mean annualized anti-VEGF injection rate of about 87% or more compared to the mean annualized anti-VEGF injection rate prior to administration of the unit dose of rAAV particles. In some embodiments, administration of the unit dose of rAAV particles to the one eye and/or to the contralateral eye of a plurality of individuals results in a reduction in the mean annualized anti-VEGF injection rate of 100% compared to the mean annualized anti-VEGF injection rate prior to administration of the unit dose of rAAV particles.

In some embodiments, the mean annualized anti-VEGF injection rate prior to administration of the unit dose of rAAV particles is calculated according to the formula:

Annualized rate prior to administration of the unit dose of rAAV particles=(number of anti-VEGF injections in 12 months prior to administration of the unit dose of rAAV particles)/(days from the first anti-VEGF injection in the past 12 months prior to administration of the unit dose of rAAV particles to the administration of the unit dose of rAAV particles/365.25)

In some embodiments, the mean annualized anti-VEGF injection rate after administration of the unit dose of rAAV particles is calculated according to the formula:

Annualized rate after administration of the unit dose of rAAV particles=(number of anti-VEGF injections since administration of the unit dose of rAAV particles)/(days from administration of the unit dose of rAAV particles/365.25).

In some embodiments, an individual is determined to require a rescue treatment (e.g., anti-VEGF intravitreal injection, such as aflibercept injection) after administration of the rAAV particles if the individual exhibits loss of 10 or more letters in BCVA (e.g., using the ETDRS protocol) in the one eye and/or the contralateral eye administered the rAAV particles compared to the BCVA in the one eye and/or the contralateral eye administered the rAAV particles prior to administration of the rAAV particles. In some embodiments, an individual is determined to require a rescue treatment (e.g., anti-VEGF intravitreal injection, such as aflibercept injection) after administration of the rAAV particles if the individual exhibits vision-threatening hemorrhage due to AMD in the one eye and/or the contralateral eye administered the rAAV particles.

In some embodiments, a rescue treatment comprises administration of a standard of care anti-VEGF therapy. Such standard of care anti-VEGF therapy comprises one or more anti-VEGF treatments (e.g., anti-VEGF intravitreal injections). In some embodiments, a rescue treatment comprises one or more aflibercept IVT injections. In some embodiments, a rescue treatment comprises one or more aflibercept IVT injections comprising about 2 mg of aflibercept.

In some embodiments, treatment (such as progress of treatment) of an ocular disease in an individual after administration of the unit dose of rAAV particles in the one eye and/or the contralateral eye is assessed based on the resolution of pigment epithelial detachment (PED) compared to PED prior to administration of the unit dose of rAAV particles in the one eye and/or the contralateral eye. In some embodiments, treatment of an ocular disease in an individual after administration of the unit dose of rAAV particles in the one eye and/or the contralateral eye is determined if resolution of PED after administration of the unit dose of rAAV particles in the one eye and/or the contralateral eye is observed, compared to PED prior to administration of the unit dose of rAAV particles in the one eye and/or the contralateral eye. In some embodiments, the ocular disease is glaucoma (e.g., neovascular glaucoma).

In some embodiments, treatment (such as progress of treatment) of an ocular disease in an individual after administration of the unit dose of rAAV particles in the one eye and/or the contralateral eye is assessed based on choroidal neovascularization (CNV) lesion growth as determined by fluorescein angiography. In some embodiments, treatment of an ocular disease in an individual after administration of the unit dose of rAAV particles in the one eye and/or the contralateral eye is determined if CNV lesions shrink (e.g., by more than any of about 5%, about 10%, about 20%, about 30%, about 40%, about 50%, about 60%, about 70%, about 80%, or 100%) after administration of the unit dose of rAAV particles in the one eye and/or the contralateral eye compared to CNV lesions present prior to administration of the unit dose of rAAV particles in the one eye and/or the contralateral eye. In some embodiments, treatment of an ocular disease in an individual after administration of the unit dose of rAAV particles in the one eye and/or the contralateral eye is determined if CNV lesions do not grow (e.g., grow less than any of about 1%, about 2%, about 3%, about 4%, about 5%, about 6%, about 7%, about 8%, about 9%, about 10%, about 15%, or about 20%) after administration of the unit dose of rAAV particles in the one eye and/or the contralateral eye compared to CNV lesions present prior to administration of the unit dose of rAAV particles in the one eye and/or the contralateral eye. In some embodiments, the ocular disease is glaucoma (e.g., neovascular glaucoma).

In some embodiments, treatment (such as progress of treatment) of an ocular disease in an individual after administration of the unit dose of rAAV particles in the one eye and/or the contralateral eye is assessed based on the anatomical features of the one eye and/or the contralateral eye based on any methods known in the art (e.g., SD-OCT, OCT, fluorescein angiography, digital color fundus photography, etc.). In some embodiments, treatment (such as progress of treatment) of an ocular disease in an individual after administration of the unit dose of rAAV particles in the one eye and/or the contralateral eye is determined if an improvement in anatomical features of the one eye and/or the contralateral eye is observed after administration of the unit dose of rAAV particles in the one eye and/or the contralateral eye. In some embodiments, the ocular disease is glaucoma (e.g., neovascular glaucoma).

In some embodiments, treatment (such as progress of treatment) of an ocular disease in an individual after administration of the unit dose of rAAV particles in the one eye and/or the contralateral eye is assessed based on ophthalmologic examination, intraocular pressure (e.g., using a Goldmann applanation tonometer or Tono-pen), indirect ophthalmoscopy, examination of the one eye and/or the contralateral eye and adnexa, eyelid and/or pupil responsiveness, belpharoptosis, abnormal pupil shape, unequal pupils, abnormal reaction to light, afferent pupillary defects, slit-lamp examination (including of the eyelids, conjunctiva, cornea, lens, iris, and anterior chamber), posterior segment abnormalities of the vitreous, optic nerve, peripheral retina, and retinal vasculature, SD-OCT, fluorescein angiography, digital color fundus photography (including images of the retina, optic disc, and/or macula), aqueous humor sampling, vitreous humor sampling, OCT-angiography (OCT-A), refraction and/or visual acuity (BCVA). In some embodiments, the ocular disease is glaucoma (e.g., neovascular glaucoma).

The unit dose of rAAV particles may be administered to the one eye and/or to the contralateral eye of the individual by any method known in the art. For example, the unit dose of rAAV particles may be administered to the one eye and/or to the contralateral eye of the individual intraocularly, or by intravitreal injection. In some embodiments, the administration of the unit dose of rAAV particles to the one eye and/or to the contralateral eye of the individual is intraocular. In some embodiments, the administration of the unit dose of rAAV particles to the one eye and/or to the contralateral eye of the individual is by intravitreal injection (IVT) or subretinal injection. In some embodiments, the administration of the unit dose of rAAV particles to the one eye and/or to the contralateral eye of the individual is by IVT injection. In some embodiments, aseptic technique is employed to administer a unit dose of rAAV particles by intravitreal injection. In some embodiments, aseptic technique with providone-iodine is employed to administer a unit dose of rAAV particles by intravitreal injection.

In some embodiments, the individual has not received a prior treatment for an ocular disease. In some embodiments, the individual has not received a prior treatment in the one eye and/or the contralateral eye for an ocular disease. In some embodiments, the individual has not received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept). In some embodiments, the individual has not received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or the contralateral eye. In some embodiments, the individual has not received a prior aflibercept treatment. In some embodiments, the individual has not received a prior aflibercept treatment in the one eye and/or the contralateral eye.

### Steroid Treatments

In some embodiments, the unit dose of rAAV particles is administered in combination with steroid treatment. In some embodiments, the steroid treatment is a corticosteroid treatment. Exemplary corticosteroids include, without limitation, aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone, and ulobetasol. In some embodiments, the steroid treatment is a systemic steroid treatment. In some embodiments, the steroid treatment is an oral steroid treatment. In some embodiments, the steroid treatment is an ophthalmic steroid treatment. In some embodiments, the ophthalmic steroid treatment is a topical steroid treatment (e.g. a drop), a periocular steroid treatment (e.g., subtenons, subconjunctival), an intravitreal steroid treatment, or a superchoroidal steroid treatment. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. In some embodiments, the ophthalmic steroid treatment is a difluprednate treatment. In some embodiments, the steroid treatment is a prednisone treatment. In some embodiments, the steroid treatment is a difluprednate treatment.

In some embodiments, the steroid treatment comprises a systemic steroid treatment and a topical steroid treatment. In some embodiments, the systemic steroid treatment is an oral steroid treatment. In some embodiments, the systemic steroid treatment is a prednisone treatment. In some embodiments, the topical steroid treatment is a difluprednate treatment. In some embodiments, the systemic steroid treatment and the topical steroid treatment are administered simultaneously (e.g., on the same day). In some embodiments, the systemic steroid treatment and the topical steroid treatment are administered separately (e.g., on different days).

In some embodiments, the steroid is administered before, during, and/or after administration of the unit dose of rAAV particles. In some embodiments, the steroid is administered before, during, and after administration of the unit dose of rAAV particles. In some embodiments, the steroid is administered during, and after administration of the unit dose of rAAV particles. In some embodiments, the steroid is administered before administration of the unit dose of rAAV particles. In some embodiments, the steroid is administered during administration of the unit dose of rAAV particles. In some embodiments, the steroid is administered before and during administration of the unit dose of rAAV particles. In some embodiments, the steroid is administered after administration of the unit dose of rAAV particles. In some embodiments, the steroid is administered during and after administration of the unit dose of rAAV particles. In some embodiments, the steroid is administered before and/or after administration of the unit dose of rAAV particles. In some embodiments, the steroid is administered before and after administration of the unit dose of rAAV particles.

In some embodiments, the steroid treatment is a systemic steroid treatment. In some embodiments, the systemic steroid treatment is an oral steroid treatment.

In some embodiments, the steroid treatment is an oral prednisone treatment. In some embodiments, the oral prednisone treatment is initiated prior to administration of the unit dose of rAAV particles. In some embodiments, an initial oral prednisone treatment is administered at a dose of any of about 40 mg, about 45 mg, about 50 mg, about 55 mg, about 60 mg, about 65 mg, or about 70 mg of prednisone per day any of about 7 days, about 6 days, about 5 days, about 4 days, about 3 days, about 2 days, about 1 day, or 0 days before administration of the unit dose of rAAV particles, and is continued for any of about 3 days, about 4 days, about 5 days, about 6 days, about 7 days, about 8 days, about 9 days, or about 10 days, or more. In some embodiments, an initial oral prednisone treatment is administered at a dose of about 60 mg of prednisone per day about 3 days before administration of the unit dose of rAAV, and is continued for about 3 days.

In some embodiments, the initial oral prednisone treatment is followed by an oral prednisone treatment dose taper. In some embodiments, the oral prednisone treatment dose taper is administered at a dose of any of about 20 mg, about 25 mg, about 30 mg, about 35 mg, about 40 mg, about 45 mg, or about 50 mg of prednisone per day for a total of any of about 1 day, about 2 days, about 3 days, about 4 days, about 5 days, about 6 days, or about 7 days, followed by a dose of about 10 mg, about 15 mg, about 20 mg or about 25 mg of prednisone per day for any of about 1 day, about 2 days, about 3 days, or about 4 days, followed by a dose of about 5 mg, about 10 mg, or about 15 mg of prednisone per day for about 1 day, about 2 days, about 3 days, or about 4 days. In some embodiments, the prednisone dose taper is administered at a dose of any of about 40 mg of prednisone per day for 3 days, followed by a dose of about 20 mg of prednisone per day for 2 days, followed by a dose of about 10 mg of prednisone per day for 2 days.

In some embodiments, an initial oral prednisone treatment is initiated 3 days before administration of the unit dose of rAAV particles at a dose of 60 mg of prednisone per day for a total of 6 days, followed by a dose of 40 mg of prednisone per day for a total of 3 days, followed by a dose of 20 mg of prednisone per day for 2 days, followed by a dose of 10 mg of prednisone per day for 2 days.

In some embodiments, the steroid treatment is an ophthalmic steroid treatment. In some embodiments, the ophthalmic steroid treatment is a difluprednate treatment. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment, e.g., a topical steroid treatment. In some embodiments, the ophthalmic steroid treatment, e.g., a topical steroid treatment, is a daily steroid treatment for up to 4 weeks, up to 6 weeks, up to 8 weeks, up to 3 months, up to 4 months, up to 5 months, or up to 6 months after administration of the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid per day (i.e., QID) for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the ophthalmic steroid treatment is extended at the discretion of the treating physician.

In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, or about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the topical steroid treatment comprises a 7-week topical steroid treatment, e.g., 0.05% difluprednate. In some embodiments, the topical steroid treatment comprises about four administrations of topical steroid per day (i.e., QID) for about four weeks, followed by about three administrations of topical steroid per day (i.e., TID) for about one week, followed by about two administrations of topical steroid per day (i.e., BID) for about one week, and followed by about one administration of topical steroid per day (i.e., QD) for about one week; timing starting at about one week prior to administration of the unit dose of rAAV particles. In some embodiments, the topical steroid treatment comprises about four administrations of topical steroid per day (i.e., QID) for about 28 days, followed by about three administrations of topical steroid per day (i.e., TID) for about 7 days, followed by about two administrations of topical steroid per day (i.e., BID) for about 7 days, and followed by about one administration of topical steroid per day (i.e., QD) for about 7 days; timing starting at about 7 days prior to administration of the unit dose of rAAV particles. In some embodiments, the topical steroid treatment comprises about four administrations of topical steroid per day (i.e., QID) on Day 1 to about Day 28, followed by about three administrations of topical steroid per day (i.e., TID) on about Day 29 to about Day 35, followed by about two administrations of topical steroid per day (i.e., BID) on about Day 36 to about Day 42, and followed by about one administration of topical steroid per day (i.e., QD) on about Day 43 to about Day 49; timing starting at Day 1. In some embodiments, the topical steroid treatment is continued if inflammation is present.

In some embodiments, the methods of treatment provided herein comprise administering an anti-VEGF agent (e.g., an aflibercept IVT injection) to one eye of the individual prior to administration of the unit dose of rAAV particles to the one eye of the individual. In some embodiments, the anti-VEGF agent is administered about 7 days or about 1 week prior to administration of the unit dose of rAAV particles. In some embodiments, the anti-VEGF agent is administered on about Day 1 and the unit dose of rAAV particles is administered on about Day 8. In some embodiments, the topical steroid treatment comprises a 7-week topical steroid treatment, e.g., 0.05% difluprednate. In some embodiments, the topical steroid treatment comprises about four administrations of topical steroid per day (i.e., QID) for about four weeks, followed by about three administrations of topical steroid per day (i.e., TID) for about one week, followed by about two administrations of topical steroid per day (i.e., BID) for about one week, and followed by about one administration of topical steroid per day (i.e., QD) for about one week; timing starting with and following administration of the anti-VEGF agent. In some embodiments, the topical steroid treatment comprises about four administrations of topical steroid per day (i.e., QID) for about 28 days, followed by about three administrations of topical steroid per day (i.e., TID) for about 7 days, followed by about two administrations of topical steroid per day (i.e., BID) for about 7 days, and followed by about one administration of topical steroid per day (i.e., QD) for about 7 days; timing starting with and following administration of the anti-VEGF agent. In some embodiments, the topical steroid treatment comprises about four administrations of topical steroid per day (i.e., QID) on Day 1 to about Day 28, followed by about three administrations of topical steroid per day (i.e., TID) on about Day 29 to about Day 35, followed by about two administrations of topical steroid per day (i.e., BID) on about Day 36 to about Day 42, and followed by about one administration of topical steroid per day (i.e., QD) on about Day 43 to about Day 49; timing starting at Day 1. In some embodiments, the topical steroid treatment is continued if inflammation is present.

In some embodiments, the topical steroid treatment comprises a 4-month topical steroid treatment, e.g., 0.05% difluprednate. In some embodiments, the topical steroid treatment comprises about four administrations of topical steroid per day (i.e., QID) for about one month, followed by about three administrations of topical steroid per day (i.e., TID) for about one month, followed by about two administrations of topical steroid per day (i.e., BID) for about one month, and followed by about one administration of topical steroid per day (i.e., QD) for about one month; timing starting at about one week prior to administration of the unit dose of rAAV particles. In some embodiments, the topical steroid treatment comprises about four administrations of topical steroid per day (i.e., QID) for about 30 days, followed by about three administrations of topical steroid per day (i.e., TID) for about 30 days, followed by about two administrations of topical steroid per day (i.e., BID) for about 30 days, and followed by about one administration of topical steroid per day (i.e., QD) for about 30 days; timing starting at about 7 days prior to administration of the unit dose of rAAV particles. In some embodiments, the topical steroid treatment comprises about four administrations of topical steroid per day (i.e., QID) on Day 1 to about Day 30, followed by about three administrations of topical steroid per day (i.e., TID) on about Day 31 to about Day 60, followed by about two administrations of topical steroid per day (i.e., BID) on about Day 61 to about Day 90, and followed by about one administration of topical steroid per day (i.e., QD) on about Day 91 to about Day 120; timing starting at Day 1. In some embodiments, the topical steroid treatment is continued if inflammation is present.

In some embodiments, the methods of treatment provided herein comprise administering an anti-VEGF agent (e.g., an aflibercept IVT injection) to one eye of the individual prior to administration of the unit dose of rAAV particles to the one eye of the individual. In some embodiments, the anti-VEGF agent is administered about 7 days or about 1 week prior to administration of the unit dose of rAAV particles. In some embodiments, the anti-VEGF agent is administered on about Day 1 and the unit dose of rAAV particles is administered on about Day 8. In some embodiments, the topical steroid treatment comprises a 4-month topical steroid treatment, e.g., 0.05% difluprednate. In some embodiments, the topical steroid treatment comprises about four administrations of topical steroid per day (i.e., QID) for about one month, followed by about three administrations of topical steroid per day (i.e., TID) for about one month, followed by about two administrations of topical steroid per day (i.e., BID) for about one month, and followed by about one administration of topical steroid per day (i.e., QD) for about one month; timing starting with and following administration of the anti-VEGF agent. In some embodiments, the topical steroid treatment comprises about four administrations of topical steroid per day (i.e., QID) for about 30 days, followed by about three administrations of topical steroid per day (i.e., TID) for about 30 days, followed by about two administrations of topical steroid per day (i.e., BID) for about 30 days, and followed by about one administration of topical steroid per day (i.e., QD) for about 30 days; timing starting with and following administration of the anti-VEGF agent. In some embodiments, the topical steroid treatment comprises about four administrations of topical steroid per day (i.e., QID) on Day 1 to about Day 30, followed by about three administrations of topical steroid per day (i.e., TID) on about Day 31 to about Day 60, followed by about two administrations of topical steroid per day (i.e., BID) on about Day 61 to about Day 90, and followed by about one administration of topical steroid per day (i.e., QD) on about Day 91 to about Day 120; timing starting at Day 1. In some embodiments, the topical steroid treatment is continued if inflammation is present.

Vectors for Delivering Transgenes to Target Cells

In some embodiments, the recombinant adeno-associated virus (rAAV) particles comprise a recombinant viral vector derived from adeno-associated virus (AAV) that has been altered so that it is replication-defective in the subject (e.g., a human or a non-human primate). In some embodiments, the adeno-associated virus (AAV) is a recombinant AAV (rAAV).

AAV or rAAV are small non-enveloped single-stranded DNA viruses. rAAVs are non-pathogenic human parvoviruses and can be made to be dependent on helper viruses, including adenovirus, herpes simplex virus, vaccinia virus and CMV, for replication.

Exposure to wild type (wt) AAV is not associated or known to cause any human pathologies and is common in the general population, making AAV or rAAV a suitable delivery system for gene therapy. AAV and rAAV used for gene therapy for delivery of an anti-VEGF agent, e.g., aflibercept, can be of any serotype. In some embodiments, the methods of the disclosure provide for use of any suitable AAV serotype, including AAV1, AAV2, AAV2.5, AAV3, AAV4, AAV5, AAV6, AAV7, AAV8, AAV9, AAV10, AAV11, AAV12, rh10, AAV-DJ, and any hybrid or chimeric AAV thereof. In some embodiments, the serotype used is based on tropism of the virus, or infectivity of a target cell of interest. In some embodiments, several AAV vectors may be generated to allow selection of the most optimal serotype for use with an anti-VEGF agent transgene (e.g., aflibercept transgene).

In some embodiments, the methods of the present disclosure provide for the use of pseudotyped AAV. Pseudotyped AAV particles comprise AAV genome inverted terminal repeats (ITRs) of one AAV serotype encapsidated by an AAV capsid of another AAV serotype. Typically, pseudotyped AAV is designated as “AAV #/#”, where the first “#” indicates the AAV ITR serotype and the second “#” indicates capsid serotype. For example, an AAV particle comprising AAV2 ITRs and an AAV1 capsid would be designated “AAV2/1”.

In some embodiments, the rAAV particles comprise a nucleic acid, e.g., a heterologous nucleic acid. In some embodiments, the nucleic acid encodes a transgene, e.g., an anti-VEGF agent (e.g., aflibercept). In some embodiments, the encoded transgene, e.g., anti-VEGF agent, is under the transcriptional control of a promoter that initiates transcription of the nucleic acid. In some embodiments, the promoter is a “ubiquitous” promoter. In some embodiments, the promoter is a “strong” or constitutively active promoter, e.g., a cytomegalovirus (CMV) promoter, an elongation factor 1 alpha (EFla) promoter, a glyceraldehyde 3-phosphate dehydrogenase (GAPDH) promoter, or a connexin36 (or “Cx36”) promoter. In some embodiments, the promoter is a tissue-specific promoter that is activated in specific tissues or cells, such as retinal cells, to reduce potential toxicity or undesirable effects to non-targeted cells. In some aspects, several AAV vectors may be generated to allow selection of the most optimal serotype and promoter for use with the anti-VEGF agent transgene (e.g., aflibercept transgene). In some embodiments, the nucleic acid is flanked by AAV inverted terminal repeats (ITRs). In some embodiments, the nucleic acid is flanked by AAV2 ITRs.

In some embodiments, the AAV vector comprises a polynucleotide cassette for enhanced expression of a transgene (e.g., an anti-VEGF agent such as aflibercept) in a target cell (e.g., a retinal cell). In some embodiments, the polynucleotide cassette comprises in 5′ to 3′ order: (a) a first enhancer region comprising a CMV sequence (SEQ ID NO: 22); (b) a promoter region, comprising a CMV sequence (SEQ ID NO: 23); (c) a 5′UTR region comprising, in 5′ to 3′ order, TPL and eMLP sequences (SEQ ID NO: 24 and SEQ ID NO: 25, respectively); (d) a coding sequence encoding a peptide or polypeptide (e.g., an anti-VEGF agent such as aflibercept); (e) a second enhancer region comprising a full EES sequence (SEQ ID NO: 26); and (f) a HGH polyadenylation site (SEQ ID NO: 27). In certain of these embodiments, the polynucleotide cassette comprises one or more sequences selected from SEQ ID NOs: 28-32, or a sequence with at least 85% identity thereto. In certain of these embodiments, the 5′ arm of the polynucleotide cassette comprises or consists of SEQ ID NO: 33 or a sequence with at least 85% identity thereto. In certain of these embodiments, the 3′ arm of the polynucleotide cassette comprises or consists of SEQ ID NO: 34 or a sequence with at least 85% identity thereto. The nucleic acid sequences of SEQ ID NOs: 22-34 are provided below:

(SEQ ID NO: 22)
ACTTACGGTA AATGGCCCGC CTGGCTGACC GCCCAACGAC CCCCGCCCAT
TGACGTCAAT AATGACGTAT GTTCCCATAG TAACGCCAAT AGGGACTTTC
CATTGACGTC AATGGGTGGA GTATTTACGG TAAACTGCCC ACTTGGCAGT
ACATCAAGTG TATCATATGC CAAGTCCGCC CCCTATTGAC GTCAATGACG
GTAAATGGCC CGCCTGGCAT TATGCCCAGT ACATGACCTT ACGGGACTTT
CCTACTTGGC AGTACATCTA CGTATTAGTC ATCGCTATTA CCA
(SEQ ID NO: 23)
TGCTGATGCG GTTTTGGCAG TACACCAATG GGCGTGGATA GCGGTTTGAC
TCACGGGGAT TTCCAAGTCT CCACCCCATT GACGTCAATG GGAGTTTGTT
TTGGCACCAA AATCAACGGG ACTTTCCAAA ATGTCGTAAT AACCCCGCCC
CGTTGACGCA AATGGGCGGT AGGCGTGTAC GGTGGGAGGT CTATATAAGC
AGAGCTCGTT TAGTGAACCG
(SEQ ID NO: 24)
CTCACTCTCT TCCGCATCGC TGTCTGCGAG GGCCAGCTGT TGGGCTCGCG
GTTGAGGACA AACTCTTCGC GGTCTTTCCA GTACTCTTGG ATCGGAAACC
CGTCGGCCTC CGAACGGTAC TCCGCCACCG AGGGACCTGA GCGAGTCCGC
ATCGACCGGA TCGGAAAACC TCTCGAGAAA GGCGTCTAAC CAGTCACAGT
CGCAAGGTAG GCTGAGCACC GTGGCGGGCG GCAGCGGGTG GCGGTCGGGG
TTGTTTCTGG CGGAGGTGCT GCTGATGATG TAATTAAAGT AGGCGGTCTT
GAGACGGCGG ATGGTCGA
(SEQ ID NO: 25)
CCAGCTGTTG GGGTGAGTAC TCCCTCTCAA AAGCGGGCAT TACTTCTGCG
CTAAGATTGT CAGTTTCCAA AAACGAGGAG GATTTGATAT TCACCTGGCC CG
(SEQ ID NO: 26)
CTGTTCTCAT CACATCATAT CAAGGTTATA TACCATCAAT ATTGCCACAG
ATGTTACTTA GCCTTTTAAT ATTTCTCTAA TTTAGTGTAT ATGCAATGAT
AGTTCTCTGA TTTCTGAGAT TGAGTTTCTC ATGTGTAATG ATTATTTAGA
GTTTCTCTTT CATCTGTTCA AATTTTTGTC TAGTTTTATT TTTTACTGAT TTGTAAGACT
TCTTTTTATA ATCTGCATAT TACAATTCTC TTTACTGGGG TGTTGCAAAT
ATTTTCTGTC ATTCTATGGC CTGACTTTTC TTAATGGTTT TTTAATTTTA
AAAATAAGTC TTAATATTCA TGCAATCTAA TTAACAATCT TTTCTTTGTG
GTTAGGACTT TGAGTCATAA GAAATTTTTC TCTACACTGA AGTCATGATG
GCATGCTTCT ATATTATTTT CTAAAAGATT TAAAGTTTTG CCTTCTCCAT
TTAGACTTAT AATTCACTGG AATTTTTTTG TGTGTATGGT ATGACATATG
GGTTCCCTTT TATTTTTTAC ATATAAATAT ATTTCCCTGT TTTTCTAAAA
AAGAAAAAGA TCATCATTTT CCCATTGTAA AATGCCATAT TTTTTTCATA
GGTCACTTAC ATATATCAAT GGGTCTGTTT CTGAGCTCTA CTCTATTTTA
TCAGCCTCAC TGTCTATCCC CACACATCTC ATGCTTTGCT CTAAATCTTG
ATATTTAGTG GAACATTCTT TCCCATTTTG TTCTACAAGA ATATTTTTGT
TATTGTCTTT GGGCTTTCTA TATACATTTT GAAATGAGGT TGACAAGTTA
(SEQ ID NO: 27)
CTGCCCGGGT GGCATCCCTG TGACCCCTCC CCAGTGCCTC TCCTGGCCCT
GGAAGTTGCC ACTCCAGTGC CCACCAGCCT TGTCCTAATA AAATTAAGTT
GCATCATTTT GTCTGACTAG GTGTCCTTCT ATAATATTAT GGGGTGGAGG
GGGGTGGTAT GGAGCAAGGG GCCCAAGTTG GGAAGAAACC TGTAGGGCCT GC
(SEQ ID NO: 28)
AGGCGGTCTT GAGACGGCGG ATGGTCGAGG TGAGGTGTGG CAGGCTTGAG
ATCCAGCTGT
TGGGGTGA
(SEQ ID NO: 29)
CGCTGTTTTG ACCTCCATAG TGGACACCGG GACCGATCCA GCCTCCGCGT
CTCAGGGGAG ATCTCGTTTA GTGAACCGTC AGATCCTCAC TCTCTTCCGC
ATCGCTGTCT GCGAGGGCCA
GCTGTTGGG
(SEQ OD NO: 30)
TTGATATTCA CCTGGCCCGA TCTGGCCATA CACTTG
(SEQ ID NO: 31)
CCCAGGTCCA AGTTTAAACG CC
(SEQ ID NO: 32)
TCTTTGGGCT TTCTATATAC ATTTTGAAAT GAGGTTGACA AGTTACCTAG
GAAAACTGTC TTCCTGCCCG GGTGGCA
(SEQ ID NO: 33)
CTCTGGAGAC GACTTACGGT AAATGGCCCG CCTGGCTGAC CGCCCAACGA
CCCCCGCCCA TTGACGTCAA TAATGACGTA TGTTCCCATA GTAACGCCAA
TAGGGACTTT CCATTGACGT CAATGGGTGG AGTATTTACG GTAAACTGCC
CACTTGGCAG TACATCAAGT GTATCATATG CCAAGTCCGC CCCCTATTGA
CGTCAATGAC GGTAAATGGC CCGCCTGGCA TTATGCCCAG TACATGACCT
TACGGGACTT TCCTACTTGG CAGTACATCT ACGTATTAGT CATCGCTATT
ACCATGCTGA TGCGGTTTTG GCAGTACACC AATGGGCGTG GATAGCGGTT
TGACTCACGG GGATTTCCAA GTCTCCACCC CATTGACGTC AATGGGAGTT
TGTTTTGGCA CCAAAATCAA CGGGACTTTC CAAAATGTCG TAATAACCCC
GCCCCGTTGA CGCAAATGGG CGGTAGGCGT GTACGGTGGG AGGTCTATAT
AAGCAGAGCT CGTTTAGTGA ACCGTCAGAT CGCCTGGAGA GGCCATCCAC
GCTGTTTTGA CCTCCATAGT GGACACCGGG ACCGATCCAG CCTCCGCGTC
TCAGGGGAGA TCTCGTTTAG TGAACCGTCA GATCCTCACT CTCTTCCGCA
TCGCTGTCTG CGAGGGCCAG CTGTTGGGCT CGCGGTTGAG GACAAACTCT
TCGCGGTCTT TCCAGTACTC TTGGATCGGA AACCCGTCGG CCTCCGAACG
GTACTCCGCC ACCGAGGGAC CTGAGCGAGT CCGCATCGAC CGGATCGGAA
AACCTCTCGA GAAAGGCGTC TAACCAGTCA CAGTCGCAAG GTAGGCTGAG
CACCGTGGCG GGCGGCAGCG GGTGGCGGTC GGGGTTGTTT CTGGCGGAGG
TGCTGCTGAT GATGTAATTA AAGTAGGCGG TCTTGAGACG GCGGATGGTC
GAGGTGAGGT GTGGCAGGCT TGAGATCCAG CTGTTGGGGT GAGTACTCCC
TCTCAAAAGC GGGCATTACT TCTGCGCTAA GATTGTCAGT TTCCAAAAAC
GAGGAGGATT TGATATTCAC CTGGCCCGAT CTGGCCATAC ACTTGAGTGA
CAATGACATC CACTTTGCCT TTCTCTCCAC AGGTGTCCAC TCCCAGGTCC
AAGTTTAAAC GCCGCCACCA TG
(SEQ ID NO: 34)
ACTGTTCTCA TCACATCATA TCAAGGTTAT ATACCATCAA TATTGCCACA
GATGTTACTT AGCCTTTTAA TATTTCTCTA ATTTAGTGTA TATGCAATGA
TAGTTCTCTG ATTTCTGAGA TTGAGTTTCT CATGTGTAAT GATTATTTAG
AGTTTCTCTT TCATCTGTTC AAATTTTTGT CTAGTTTTAT TTTTTACTGA TTTGTAAGAC
TTCTTTTTAT AATCTGCATA TTACAATTCT CTTTACTGGG GTGTTGCAAA TATTTTCTGT
CATTCTATGG CCTGACTTTT CTTAATGGTT TTTTAATTTT AAAAATAAGT
CTTAATATTC ATGCAATCTA ATTAACAATC TTTTCTTTGT GGTTAGGACT
TTGAGTCATA AGAAATTTTT CTCTACACTG AAGTCATGAT GGCATGCTTC
TATATTATTT TCTAAAAGAT TTAAAGTTTT GCCTTCTCCA TTTAGACTTA
TAATTCACTG GAATTTTTTT GTGTGTATGG TATGACATAT GGGTTCCCTT
TTATTTTTTA CATATAAATA TATTTCCCTG TTTTTCTAAA AAAGAAAAAG
ATCATCATTT TCCCATTGTA AAATGCCATA TTTTTTTCAT AGGTCACTTA
CATATATCAA TGGGTCTGTT TCTGAGCTCT ACTCTATTTT ATCAGCCTCA
CTGTCTATCC CCACACATCT CATGCTTTGC TCTAAATCTT GATATTTAGT
GGAACATTCT TTCCCATTTT GTTCTACAAG AATATTTTTG TTATTGTCTT TGGGCTTTCT
ATATACATTT TGAAATGAGG TTGACAAGTT ACCTAGGAAA ACTGTCTTCC
TGCCCGGGTG GCATCCCTGT GACCCCTCCC CAGTGCCTCT CCTGGCCCTG
GAAGTTGCCA CTCCAGTGCC CACCAGCCTT GTCCTAATAA AATTAAGTTG
CATCATTTTG TCTGACTAGG TGTCCTTCTA TAATATTATG GGGTGGAGGG
GGGTGGTATG GAGCAAGGGG CCCAAGTTGG GAAGAAACCT GTAGGGCCTG
CGAAGACAGT CAG

In some embodiments, the polynucleotide cassette comprises or consists of SEQ ID NO: 39 or a sequence with at least 85% identity thereto.

(SEQ ID NO: 39)
gcgcgctcgctcgctcactgaggccgcccgggcaaagcccgggcgtcgggcgacctttggtcgcccggcctcagtgagcgagcgagcgcgc
agagagggagtggccaactccatcactaggggttccttgtagttaatgattaacccgccatgctacttatctacgtactctggagacgacttacg
gtaaatggcccgcctggctgaccgcccaacgacccccgcccattgacgtcaataatgacgtatgttcccatagtaacgccaatagggactttcca
ttgacgtcaatgggtggagtatttacggtaaactgcccacttggcagtacatcaagtgtatcatatgccaagtccgccccctattgacgtcaatg
acggtaaatggcccgcctggcattatgcccagtacatgaccttacgggactttcctacttggcagtacatctacgtattagtcatcgctattacc
atgctgatgcggttttggcagtacaccaatgggcgtggatagcggtttgactcacggggatttccaagtctccaccccattgacgtcaatgggag
tttgttttggcaccaaaatcaacgggactttccaaaatgtcgtaataaccccgccccgttgacgcaaatgggcggtaggcgtgtacggtgggagg
tctatataagcagagctcgtttagtgaaccgtcagatcgcctggagaggccatccacgctgttttgacctccatagtggacaccgggaccgatcc
agcctccgcgtctcaggggagatctcgtttagtgaaccgtcagatcctcactctcttccgcatcgctgtctgcgagggccagctgttgggctcgc
ggttgaggacaaactcttcgcggtctttccagtactcttggatcggaaacccgtcggcctccgaacggtactccgccaccgagggacctgagcga
gtccgcatcgaccggatcggaaaacctctcgagaaaggcgtctaaccagtcacagtcgcaaggtaggctgagcaccgtggcgggcggcagcgggt
ggcggtcggggttgtttctggcggaggtgctgctgatgatgtaattaaagtaggcggtcttgagacggcggatggtcgaggtgaggtgtggcagg
cttgagatccagctgttggggtgagtactccctctcaaaagcgggcattacttctgcgctaagattgtcagtttccaaaaacgaggaggatttga
tattcacctggcccgatctggccatacacttgagtgacaatgacatccactttgcctttctctccacaggtgtccactcccaggtccaagtttaa
acgccgccaccatggtgtcatactgggatactggagtcttgctttgtgccctgctgtcctgcctcctcctgactggctccagctcgggctcagat
accggtcgccccttcgtggagatgtactccgagatcccggaaattatccacatgactgaggggcgcgaacttgtgatcccctgccgggtcaccag
cccgaacattactgtgactttgaagaagttccccctggacaccctgattccggatgggaagagaattatctgggattcacggaagggattcatca
tcagcaacgcgacctacaaggaaattggcctcctcacttgcgaagccactgtgaacggacacttgtacaagaccaactacctgacccaccgccag
accaacaccatcatcgacgtcgtcctgtccccttcgcacgggatcgagctctcggtgggagagaagttggtgcttaactgcaccgcccggacgga
actgaatgtgggaatcgacttcaactgggaatacccgtccagcaagcatcagcataagaagctggtgaaccgggacctcaagactcagtccggca
gcgaaatgaagaagttcctgtcgaccctcactattgacggagtgaccagatccgaccagggcctctacacttgcgccgcttccagcggactcatg
accaagaagaacagcactttcgtgagggtgcatgagaaggacaagacccacacgtgtccgccgtgcccagccccagagctgctgggaggcccttc
cgtgttcctgtttccgcccaagccaaaggataccctgatgatctcaaggacccctgaggtcacatgcgtcgtggtggatgtgtcgcacgaggacc
ctgaagtcaaattcaattggtatgtggacggagtggaagtccacaacgcgaaaaccaagccgagagaagaacagtacaattccacctaccgggtg
gtgtcggtgctgactgtgctgcaccaggactggctcaacggaaaggagtacaagtgcaaggtgtccaacaaggctctgcccgcacctattgaaaa
gaccatctccaaggccaagggtcaacctcgcgagcctcaggtgtacactctgcctccaagccgggacgaactgactaagaaccaagtctctctga
cctgtttggtgaagggcttctacccgtcagacatcgcagtggagtgggagtcaaacggtcagccggagaacaactacaaaacaaccccccccgtg
ctggactccgacggctccttcttcctgtactccaagcttaccgtggataagagccgctggcaacagggcaacgtgttttcctgctccgtcatgca
cgaagccctgcacaaccattatacccagaagtccctgtcgctgtcccccgggaaatagtgactgttctcatcacatcatatcaaggttatatacc
atcaatattgccacagatgttacttagccttttaatatttctctaatttagtgtatatgcaatgatagttctctgatttctgagattgagtttct
catgtgtaatgattatttagagtttctctttcatctgttcaaatttttgtctagttttattttttactgatttgtaagacttctttttataatct
gcatattacaattctctttactggggtgttgcaaatattttctgtcattctatggcctgacttttcttaatggttttttaattttaaaaataagt
cttaatattcatgcaatctaattaacaatcttttctttgtggttaggactttgagtcataagaaatttttctctacactgaagtcatgatggcat
gcttctatattattttctaaaagatttaaagttttgccttctccatttagacttataattcactggaatttttttgtgtgtatggtatgacatat
gggttcccttttattttttacatataaatatatttccctgtttttctaaaaaagaaaaagatcatcattttcccattgtaaaatgccatattttt
ttcataggtcacttacatatatcaatgggtctgtttctgagctctactctattttatcagcctcactgtctatccccacacatctcatgctttgc
tctaaatcttgatatttagtggaacattctttcccattttgttctacaagaatatttttgttattgtctttgggctttctatatacattttgaaa
tgaggttgacaagttacctaggaaaactgtcttcctgcccgggtggcatccctgtgacccctccccagtgcctctcctggccctggaagttgcca
ctccagtgcccaccagccttgtcctaataaaattaagttgcatcattttgtctgactaggtgtccttctataatattatggggtggaggggggtg
gtatggagcaaggggcccaagttgggaagaaacctgtagggcctgcgtacgtagataagtagcatggcgggttaatcattaactacaaggaaccc
ctagtgatggagttggccactccctctctgcgcgctcgctcgctcactgaggccgggcgaccaaaggtcgcccgacgcccgggctttgcccgggc
ggcctcagtgagcgagcgagcgcgc

SEQ ID NO: 39 shown above comprises, in the 5′ to 3′ direction, an inverted terminal repeat (ITR) of AAV serotype 2 comprising nucleotides 1-145 of SEQ ID NO: 39; a CMV promoter comprising nucleotides 180-693 of SEQ ID NO: 39; a 5′ Untranslated Region (UTR), including an Adenovirus Tripartite Leader Sequence and Synthetic Intron, and comprising nucleotides 694-1314 of SEQ ID NO: 39; a Kozak sequence comprising nucleotides 1329-1340 of SEQ ID NO: 39; a codon-optimized aflibercept cDNA sequence comprising nucleotides 1338-2714 of SEQ ID NO: 39; a 3′ UTR including a human scaffold attachment region and comprising nucleotides 2717-3527 of SEQ ID NO: 39; a human growth hormone polyadenylation/transcription stop signal comprising nucleotides 3546-3748 of SEQ ID NO: 39; and an inverted terminal repeat (ITR) of AAV serotype 2 comprising nucleotides 3772-3916 of SEQ ID NO: 39.

Additional polynucleotide cassettes for enhanced expression of a transgene (e.g., a transgene encoding an anti-VEGF agent such as aflibercept) in a target cell (such as a retinal cell) are disclosed in WO2018/170473, the contents of which related to polynucleotide cassettes for enhanced expression of a transgene in a target cell are incorporated herein by reference.

In some aspects, the invention provides methods for treating glaucoma in an individual, the method comprising administering a unit dose of recombinant adeno-associated virus (rAAV) particles to one eye of the individual, wherein the individual is a human, and wherein the rAAV particles comprise a nucleic acid encoding a polypeptide comprising an amino acid sequence with at least about 95% identity to the amino acid sequence of SEQ ID NO: 35 and flanked by AAV2 inverted terminal repeats (ITRs). In some aspects, the invention provides a method for reducing intraocular pressure in an individual, the method comprising administering a unit dose of recombinant adeno-associated virus (rAAV) particles to one eye of the individual, wherein the individual is a human, and wherein the rAAV particles comprise a nucleic acid encoding a polypeptide comprising an amino acid sequence with at least about 95% identity to the amino acid sequence of SEQ ID NO: 35 and flanked by AAV inverted terminal repeats (ITRs). In some embodiments, the methods of the disclosure provide for use of any suitable AAV serotype, including AAV1, AAV2, AAV2.5, AAV3, AAV4, AAV5, AAV6, AAV7, AAV8, AAV9, AAV10, AAV11, AAV12, rh10, AAV-DJ, and any hybrid or chimeric AAV thereof.

In some embodiments, the rAAV particles comprise a variant capsid protein having increased infectivity of target cells, e.g. retinal cells, are used to increase transduction of retinal cells or to increase targeting of gene delivery to retinal cells in an individual. In some embodiments, the rAAV particle comprises an amino acid modification in a capsid protein GH loop/loop IV of the AAV capsid protein. In some embodiments, the site of modification is a solvent-accessible portion of the GH loop/loop IV of the AAV capsid protein. For a description of the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. Several AAV capsid variants are known, including the 7m8 variant. In some embodiments, a rAAV particle comprises a variant AAV capsid protein that comprises an insertion of from 5 amino acids to 11 amino acids, e.g., 7 amino acid sequence, in the GH loop of a capsid protein relative to a corresponding parental AAV capsid protein, and wherein the variant capsid protein confers increased infectivity of a retinal cell compared to the infectivity of the retinal cell by an AAV particle comprising the corresponding parental or unmodified AAV capsid protein. In some embodiments, any one of the following amino acid sequences can be inserted in the GH loop of a capsid protein: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21). In some embodiments, any one of the amino acid sequences set forth in SEQ ID NOs: 1-12 and 14-21 is inserted in the solvent-exposed GH loop of VP1 capsid protein in a rAAV. Additional details regarding amino acid sequences that can be inserted into the GH loop of a capsid protein, e.g., to facilitate transduction of a nucleic acid of interest to a retinal cell following IVT injection, are provided in WO2012145601, U.S. Pat. No. 9,587,282, U.S. Ser. No. 10/202,657, and U.S. Ser. No. 10/214,785, the contents of which related to amino acid sequences that can be inserted into the GH loop of a capsid protein are incorporated herein by reference.

In some embodiments, the rAAV particles comprise an AAV capsid protein, e.g., an AAV2 capsid protein, that includes any one of the following amino acid sequences: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21) inserted at the following positions: between positions 587 and 588 of the AAV2 capsid protein; between amino acids 590 and 591 of the AAV1 capsid protein; between amino acids 575 and 576 of the AAV5 capsid protein; between amino acids 590 and 591 of the AAV6 capsid protein; between amino acids 589 and 590 of the AAV7 capsid protein; between amino acids 590 and 591 of the AAV8 capsid protein; between amino acids 588 and 589 of the AAV9 capsid protein; or between amino acids 589 and 590 of the AAV10 capsid protein. In some embodiments, the rAAV particles comprise AAV2 capsid proteins comprising an amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise AAV2 capsid proteins comprising the amino acid sequence LALGETTRPA (SEQ ID NO: 1) inserted between positions 587 and 588 of the AAV2 VP1 comprising the sequence of SEQ ID NO: 13.

In some embodiments, rAAV particles comprise the 7m8 variant capsid protein from AAV2 comprising the amino acid sequence LALGETTRPA (SEQ ID NO: 1) inserted in the GH loop of the AAV2 VP1 protein between positions 587 and 588 of the AAV2 VP1. In some embodiments, the rAAV particles comprise an AAV2 VP1 capsid protein comprising a GH loop that comprises the amino acid sequence of SEQ ID NO: 38 or an amino acid sequence having at least 90% sequence identity to SEQ ID NO: 38. In some embodiments, the rAAV particles comprise an AAV2 VP1 capsid protein comprising a GH loop that comprises an amino acid sequence having any of at least 90%, at least 91%, at least 92%, at least 93%, at least 94%, at least 95%, at least 96%, at least 97%, at least 98%, at least 99%, or 100% sequence identity to SEQ ID NO: 38.

(SEQ ID NO: 38)
FSYTFEDVPFHSSYAHSQSLDRLMNPLIDQYLYYLSRTNTPSGTTTQSRL
QFSQAGASDIRDQSRNWLPGPCYRQQRVSKTSADNNNSEYSWTGATKYHL
NGRDSLVNPGPAMASHKDDEEKFFPQSGVLIFGKQGSEKTNVDIEKVMIT
DEEEIRTTNPVATEQYGSVSTNLQRGNLALGETTRPARQAATADVNTQGV
LPGMVWQDRDVYLQGPIWAKIPHTDGHFHPSPLMGGFGLKHPPPQILIKN

In some embodiments, rAAV particles comprise the 7m8 variant capsid protein from AAV2 comprising the amino acid sequence LALGETTRPA (SEQ ID NO: 1) inserted between positions 587 and 588 of the AAV2 VP1. The sequence of the 7m8 variant capsid protein from AAV2 comprising the amino acid sequence LALGETTRPA (SEQ ID NO: 1) is inserted between positions 587 and 588 of the AAV2 VP1 is provided below:

(SEQ ID NO: 37)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNLALGETTRPARQA
ATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKEN
SKRWNPEIQYTSNYNKSVNVDFTVDINGVYSEPRPIGTRYLTRNL

In some embodiments, the rAAV particles comprise a capsid protein VP1 comprising the amino acid sequence LGETTRP (SEQ ID NO: 14) inserted between positions 587 and 588 of the capsid protein, wherein the amino acid residue numbering corresponds to an AAV2 VP1 capsid protein. In some embodiments, the rAAV particles comprise a capsid protein VP2 comprising the amino acid sequence LGETTRP (SEQ ID NO: 14) inserted between positions 587 and 588 of the capsid protein, wherein the amino acid residue numbering corresponds to an AAV2 VP1 capsid protein. In some embodiments, the rAAV particles comprise a capsid protein VP3 comprising the amino acid sequence LGETTRP (SEQ ID NO: 14) inserted between positions 587 and 588 of the capsid protein, wherein the amino acid residue numbering corresponds to an AAV2 VP1 capsid protein. In some embodiments, the rAAV particles comprise capsid proteins VP1, VP2, and VP3, wherein each of VP1, VP2, and VP3 comprise the amino acid sequence LGETTRP (SEQ ID NO: 14) inserted between positions 587 and 588 of the capsid protein, wherein the amino acid residue numbering corresponds to an AAV2 VP1 capsid protein.

In some embodiments, the rAAV particles comprise a capsid protein VP1 comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise a capsid protein VP2 comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise a capsid protein VP3 comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise capsid proteins VP1, VP2, and VP3, wherein each of VP1, VP2, and VP3 comprise the amino acid sequence LALGETTRPA (SEQ ID NO: 1) inserted between positions 587 and 588 of the capsid protein, wherein the amino acid residue numbering corresponds to an AAV2 VP1 capsid protein.

In some embodiments, a recombinant virus and/or plasmid used to generate a rAAV virus comprises other transcriptional or regulatory elements, such as a poly A (polyadenylation) sequence, untranslated regions (UTRs), 3′ UTRs, or termination sequences. In some embodiments, more than one gene is expressed from the vector or plasmid using internal ribosome entry site (IRES) or similar element that allows co-expression of two or more proteins or create multigene, or polycistronic mRNA.

In some embodiments, the rAAV and/or plasmid used to generate the rAAV comprises one or more of the following nucleic acid elements: a first ITR sequence; a promoter sequence; an intron sequence; a first UTR sequence; a heterologous nucleic acid encoding an anti-VEGF agent (e.g., aflibercept); a second UTR sequence; a polyA sequence; and a second ITR sequence. In some embodiments, linker sequence(s) are inserted between two or more of the nucleic acid elements. In some embodiments, the heterologous nucleic acid encodes a therapeutic polypeptide, e.g., encodes aflibercept (or a functional fragment or functional variant thereof).

In some embodiments, the vector is a targeted vector, especially a targeted rAAV (e.g., AAV2.7m8) that shows higher infectivity of a specific cell, such as a retinal cell (e.g., a photoreceptor, a retinal ganglion cell, a Müller cell, a bipolar cell, an amacrine cell, a horizontal cell, or a retinal pigmented epithelium cell). Viral vectors for use in the disclosure can include those that exhibit low toxicity and/or low immunogenicity in an individual and expresses therapeutically effective quantities of the anti-VEGF agent (e.g., aflibercept) in an individual, e.g., a human. Any suitable method known in the art can be used in the biochemical purification of recombinant viruses (e.g., rAAV), e.g., for the preparation of pharmaceutical compositions described elsewhere herein. Recombinant AAV viruses can be harvested directly from cells, or from the culture media comprising cells. Virus can be purified using various biochemical means, such as gel filtration, filtration, chromatography, affinity purification, gradient ultracentrifugation, or size exclusion methods. In some embodiments, the virus is lyophilized.

In some embodiments, the rAAV particles comprise a 7m8 variant capsid protein, e.g., rAAV2.7m8, and a nucleic acid sequence that encodes an anti-VEGF agent (e.g., aflibercept, or a functional fragment or functional variant thereof). In some embodiments, the rAAV particles (e.g., the 7m8 variant) have an increase in retinal cell infectivity of any of at least 5%, at least 10%, at least 20%, at least 30%, at least 40%, at least 50%, at least 60%, at least 70%, at least 80%, at least 90%, or at least 100% as compared to an AAV particle comprising the corresponding parental or unmodified AAV capsid protein. In some embodiments, the increase in infectivity of retinal cells is an increase of any of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, or between 5% to 10%, as compared to an AAV particle comprising the corresponding parental or unmodified AAV capsid protein.

In some embodiments, the increase in retinal cell infectivity of a rAAV variant, e.g., rAAV2.7m8, is any of at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV particle comprising the corresponding parental or unmodified AAV capsid protein. In some embodiments, the increase in infectivity is any of at least 2-fold, at least 3-fold, at least 4-fold, at least 5-fold, at least 6-fold, at least 7-fold, at least 8-fold, at least 9-fold, or at least 10-fold, as compared to an AAV particle comprising the corresponding parental AAV capsid protein. In some embodiments, the increase in infectivity is any of at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV particle comprising the corresponding parental or unmodified AAV capsid protein.

In some embodiments, the increase in retinal cell infectivity of a rAAV variant, e.g., rAAV2.7m8, is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between 10-fold to 25-fold, between 10-fold to 20-fold, or between 10-fold to 15-fold, as compared to an AAV particle comprising the corresponding parental or unmodified AAV capsid protein.

In some embodiments, the increase in retinal cell infectivity is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold, as compared to an AAV particle comprising the corresponding parental or unmodified AAV capsid protein.

In some embodiments, an amino acid modification of a capsid protein described herein can confer an increase in an ability to cross an internal limiting membrane (ILM) in an eye of an individual, e.g., a human, as compared to the ability of an AAV particle comprising the corresponding parental or unmodified AAV capsid protein to cross the ILM in the eye of the subject. In some embodiments, the increase in the ability to cross the TLM of a rAAV variant, e.g., rAAV2.7m8, is an increase of any of at least 5%, at least 10%, at least 20%, at least 30%, at least 40%, at least 50%, at least 60%, at least 70%, at least 80%, at least 90%, or at least 100% as compared to an AAV particle comprising the corresponding parental or unmodified AAV capsid protein. In some embodiments, the increase in the ability to cross the ILM is an increase of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, or between 5% to 10%, as compared to the parental or unmodified AAV capsid protein.

In some embodiments, the increase in the ability to cross the TLM of a rAAV variant, e.g., rAAV2.7m8, is any of at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV particle comprising the corresponding parental AAV capsid protein. In some embodiments, the increase in the ability to cross the ILM is any of at least 2-fold, at least 3-fold, at least 4-fold, at least 5-fold, at least 6-fold, at least 7-fold, at least 8-fold, at least 9-fold, or at least 10-fold, as compared to an AAV particle comprising the corresponding parental AAV capsid protein. In some embodiments, the increase in the ability to cross the ILM is any of at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV particle comprising the corresponding parental or unmodified AAV capsid protein.

In some embodiments, the increase in the ability to cross the ILM of a rAAV variant, e.g., rAAV2.7m8, is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between 10-fold to 25-fold, between 10-fold to 20-fold, or between 10-fold to 15-fold as compared to an AAV particle comprising the corresponding parental or unmodified AAV capsid protein.

In some embodiments, the increase in the ability to cross the ILM of a rAAV variant, e.g., rAAV2.7m8, is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold, as compared to an AAV particle comprising the corresponding parental or unmodified AAV capsid protein.

In some embodiments, rAAV.7m8 comprising nucleic acid encoding aflibercept is used for gene therapy. In some embodiments, AAV2 or rAAV2 is used to deliver a nucleic acid sequence encoding an anti-VEGF agent (e.g., aflibercept) into an eye or retinal cells of a subject via intravitreal or subretinal injection. In some embodiments, AAV2 or rAAV2 is used to deliver a nucleic acid sequence encoding an anti-VEGF agent (e.g., aflibercept) into an eye or retinal cells of a subject via intravitreal injection. In some embodiments, rAAV2.7m8 is used to deliver the nucleic acid sequence of the anti-VEGF agent (e.g., aflibercept) into the retinal cells of a subject. In some embodiments, the heterologous nucleic acid (e.g., a nucleic acid that encodes an anti-VEGF agent such as aflibercept) integrates into the target cell genome (e.g., retinal cell genome), resulting in long-term expression of, e.g., the anti-VEGF agent (such as aflibercept), in the target cell. In some embodiments, the viral vector delivers a plasmid or other extrachromosomal genetic element that comprises the heterologous nucleic acid (e.g., a nucleic acid that encodes an anti-VEGF agent such as aflibercept) to the target cell (e.g., retinal cell).

In some embodiments, the rAAV particles comprise a nucleic acid encoding a polypeptide comprising an amino acid sequence with any of at least about 95%, at least about 96%, at least about 97%, at least about 98%, at least about 99%, or at least about 100% identity to the amino acid sequence of SEQ ID NO: 35 and flanked by AAV2 inverted terminal repeats (ITRs). In some embodiments, the rAAV particles comprise a nucleic acid encoding a polypeptide comprising an amino acid sequence with at least about 95% identity to the amino acid sequence of SEQ ID NO: 35 and flanked by AAV2 inverted terminal repeats (ITRs). In some embodiments, the rAAV particles comprise a nucleic acid encoding a polypeptide comprising the amino acid sequence of SEQ ID NO: 35 and flanked by AAV2 inverted terminal repeats (ITRs). In some embodiments, the rAAV particles comprise a nucleic acid encoding a polypeptide comprising the amino acid sequence of SEQ ID NO: 35. In some embodiments, the rAAV particles comprise a nucleic acid encoding aflibercept and flanked by AAV2 inverted terminal repeats (ITRs). The sequence of SEQ ID NO: 35 is provided below:

(SEQ ID NO: 35)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG

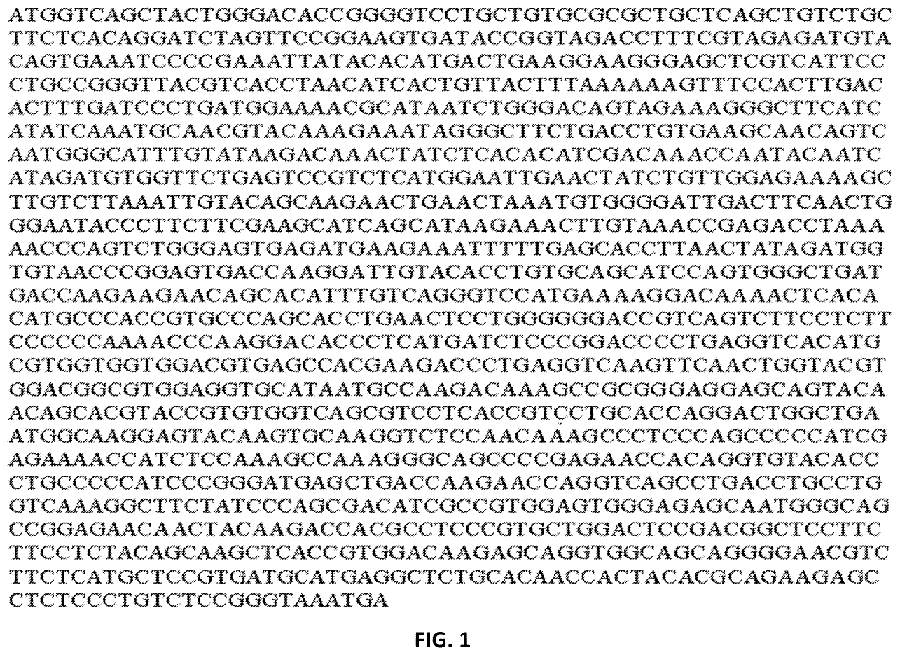

In some embodiments, the rAAV particles comprise a nucleic acid with any of at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.9%, or at least about 100% sequence homology to the nucleic acid sequence of SEQ ID NO: 36, and wherein the nucleic acid is flanked by AAV2 inverted terminal repeats (ITRs). The sequence of SEQ ID NO: 36 is provided in FIG. 1. In some embodiments, the rAAV particles comprise a nucleic acid with any of at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.9%, or at least about 100% sequence homology to the nucleic acid sequence of aflibercept (e.g., SEQ ID NO: 36), and wherein the nucleic acid is flanked by AAV2 inverted terminal repeats (ITRs). In some embodiments, the nucleic acid sequence of aflibercept is derived from its amino acid sequence. In some embodiments, the nucleic acid sequence of aflibercept is codon optimized to improve its expression in a subject. In some embodiments, the rAAV particles comprise a nucleic acid with any of at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.9%, or 100% sequence homology to the nucleic acid sequence of SEQ ID NO: 40, and wherein the nucleic acid is flanked by AAV2 inverted terminal repeats (ITRs). In some embodiments, the rAAV particles comprise a nucleic acid comprising the nucleic acid sequence of SEQ ID NO: 40. In some embodiments, the rAAV particles comprise a nucleic acid comprising the nucleic acid sequence of SEQ ID NO: 40, and wherein the nucleic acid is flanked by AAV2 inverted terminal repeats (ITRs).

(SEQ ID NO: 40)
atggtgtcatactgggatactggagtcttgctttgtgccctgctgtcctgcctcctcctgactggctccagctcgggctcagataccggtcgccc
cttcgtggagatgtactccgagatcccggaaattatccacatgactgaggggcgcgaacttgtgatcccctgccgggtcaccagcccgaacatta
ctgtgactttgaagaagttccccctggacaccctgattccggatgggaagagaattatctgggattcacggaagggattcatcatcagcaacgcg
acctacaaggaaattggcctcctcacttgcgaagccactgtgaacggacacttgtacaagaccaactacctgacccaccgccagaccaacaccat
catcgacgtcgtcctgtccccttcgcacgggatcgagctctcggtgggagagaagttggtgcttaactgcaccgcccggacggaactgaatgtgg
gaatcgacttcaactgggaatacccgtccagcaagcatcagcataagaagctggtgaaccgggacctcaagactcagtccggcagcgaaatgaag
aagttcctgtcgaccctcactattgacggagtgaccagatccgaccagggcctctacacttgcgccgcttccagcggactcatgaccaagaagaa
cagcactttcgtgagggtgcatgagaaggacaagacccacacgtgtccgccgtgcccagccccagagctgctgggaggcccttccgtgttcctgt
ttccgcccaagccaaaggat
accctgatgatctcaaggacccctgaggtcacatgcgtcgtggtggatgtgtcgcacgaggaccctgaagtcaaattcaattggtatgtggacgg
agtggaagtccacaacgcgaaaaccaagccgagagaagaacagtacaattccacctaccgggtggtgtcggtgctgactgtgctgcaccaggac
tggctcaacggaaaggagtacaagtgcaaggtgtccaacaaggctctgcccgcacctattgaaaagaccatctccaaggccaagggtcaacctc
gcgagcctcaggtgtacactctgcctccaagccgggacgaactgactaagaaccaagtctctctgacctgtttggtgaagggcttctacccgtca
gacatcgcagtggagtgggagtcaaacggtcagccggagaacaactacaaaacaaccccccccgtgctggactccgacggctccttcttcctgta
ctccaagcttaccgtggataagagccgctggcaacagggcaacgtgttttcctgctccgtcatgcacgaagccctgcacaaccattatacccaga
agtccctgtcgctgtcccccgggaaatag

In some embodiments, the rAAV particles comprise a nucleic acid encoding a polypeptide comprising an amino acid sequence with any of at least about 95%, at least about 96%, at least about 97%, at least about 98%, at least about 99%, or 100% identity to the amino acid sequence of SEQ ID NO: 41 and flanked by AAV2 inverted terminal repeats (ITRs). In some embodiments, the rAAV particles comprise a nucleic acid encoding a polypeptide comprising an amino acid sequence with at least about 95% identity to the amino acid sequence of SEQ ID NO: 41 and flanked by AAV2 inverted terminal repeats (ITRs). In some embodiments, the rAAV particles comprise a nucleic acid encoding a polypeptide comprising the amino acid sequence of SEQ ID NO: 41 and flanked by AAV2 inverted terminal repeats (ITRs). In some embodiments, the rAAV particles comprise a nucleic acid encoding a polypeptide comprising the amino acid sequence of SEQ ID NO: 41.

(SEQ ID NO: 41)
MVSYWDTGVLLCALLSCLLLTGSSSGSDTGRPFVEMYSEIPEIIHMTEGR
ELVIPCRVTSPNITVTLKKFPLDTLIPDGKRIIWDSRKGFIISNATYKEI
GLLTCEATVNGHLYKTNYLTHRQTNTIIDVVLSPSHGIELSVGEKLVLNC
TARTELNVGIDENWEYPSSKHQHKKLVNRDLKTQSGSEMKKFLSTLTIDG
VTRSDQGLYTCAASSGLMTKKNSTFVRVHEKDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

In some embodiments, the nucleic acid sequence of aflibercept is codon-optimized for expression in a primate or a human subject. Construction of a synthetic gene corresponding to the aflibercept amino acid sequence has been described in literature, e.g., Kanda A, Noda K, Saito W, Ishida S. Aflibercept Traps Galectin-1, an Angiogenic Factor Associated with Diabetic Retinopathy. Scientific Reports 5:17946 (2015) (describing “VEGF-TrapR1R2 (corresponding to aflibercept) cDNA was generated as a synthetic gene by IDT (Coralville, IA)”). Given the available amino acid sequence of aflibercept, any method known in the art can be used to generate the cDNA of aflibercept for use in a gene therapy or a rAAV described herein.

Codon optimization can be achieved with any method known in the art. Codon optimization refers to a process of modifying a nucleic acid sequence for enhanced expression of a gene in target or host cells of interest, e.g., human retinal cells, by replacing at least one codon (e.g., about or more than 1, 2, 3, 4, 5, 10, 15, 20, 25, 50, 100 or more codons) of a native sequence with codons that are used more frequently or are most frequently used in the host cell while maintaining the native amino acid sequence. Codon usage tables are readily available, including for examples, GenScript Codon Usage Frequency Table Tool at www(dot)genscript(dot)com/tools/codon-frequency-table; Codon Usage Database at www(dot)kazusa(dot)or(dot)jp/codon/; and Nakamura, Y., et al. “Codon usage tabulated from the international DNA sequence databases: status for the year 2000” Nucl. Acids Res. 28:292 (2000).

Homology refers to the percent conservation of residues of an alignment between two sequences, including, but not limited to functional fragments, sequences comprising insertions, deletions, substitutions, pseudofragments, pseudogenes, splice variants or artificially optimized sequences.

In some embodiments, the rAAV particles comprise a nucleic acid encoding aflibercept. In some embodiments, the polypeptide is aflibercept.

As used herein, “aflibercept” refers to a polypeptide or protein sequence, or a functional fragment or variant or mutant thereof, with any of at least 75%, 80%, 81%, 82%, 83%, 84%, 85%, 86%, 87%, 88%, 89%, 90%, 91%, 92%, 93%, 94%, 95%, 96%, 97%, 98%, 99% or more, or 100% homology to the aflibercept amino acid sequence identified above (SEQ ID NO: 35). Homology refers to the percent conservation of residues of an alignment between two sequences, including, but not limited to functional fragments, sequences comprising insertions, deletions, substitutions, pseudofragments, pseudogenes, splice variants or artificially optimized sequences.

In some embodiments, the amino acid sequence of aflibercept is any of at least 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% homologous to the aflibercept amino acid sequence of SEQ ID NO: 35. In some embodiments, the nucleic acid sequence encoding aflibercept disclosed herein is compared to the corresponding cDNA sequence of the aflibercept amino acid sequence identified above, and shows any of at least 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence homology between the nucleic acid sequences of aflibercept (e.g., SEQ ID NO: 36). In some embodiments, aflibercept is any of at least 80%, 85%, 90%, 91%, 92%, 93%, 94%, 95%, 96%, 97%, 98%, 99%, 99.9%, or 100% spatially homologous to aflibercept (e.g., in terms of its secondary, tertiary, and quaternary structure or conformation). In some embodiments, aflibercept is any of at most 80%, 85%, 90%, 91%, 92%, 93%, 94%, 95%, 96%, 97%, 98%, 99%, 99.9%, or 100% spatially homologous to the aflibercept used in the standard of care (e.g., secondary, tertiary, and quaternary structure or conformation).

In some embodiments, the aflibercept gene product, or aflibercept transgene, as included in a gene therapy based on a rAAV, comprises a capsid variant as disclosed herein (e.g., the 7m8 variant), encodes a protein, fusion protein, or polypeptide that has any of at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% homology to the above amino acid sequence of SEQ ID NO: 35, or between the corresponding cDNA sequences of aflibercept (e.g., cDNA of aflibercept sequence used in a gene therapy compared to SEQ ID NO: 36). In some embodiments, the methods compositions disclosed herein comprise a functional fragment of aflibercept, or a variant or mutant thereof. In some embodiments, the nucleic acid sequence of aflibercept is modified or codon-optimized to enhance its activity, expression, stability, and/or solubility in vivo.

Aflibercept is a 115 kDa fusion protein, which can be glycosylated. Aflibercept comprises an IgG backbone fused to extracellular VEGF receptor sequences of the human VEGFR-1 and VEGFR-2, and functions like a soluble decoy receptor by binding VEGF-A with a greater affinity than its natural or endogenous receptors. See, for example, Stewart MW. Aflibercept (VEGF Trap-eye): the newest anti-VEGF drug. Br. J. Ophthalmol. 2012 September; 96(9):1157-8. Aflibercept's high affinity for VEGF interferes or disrupts subsequent binding and activation of native or endogenous VEGF receptors. Reduced VEGF activity can lead to decreased angiogenesis and vascular permeability. Inhibition of placental growth factor PIGF and VEGF-B by aflibercept may also contribute to the treatment of ocular diseases or disorders characterized by abnormal (e.g., excessive) angiogenesis and/or neovascularization. PIGF has been associated with angiogenesis and certain ocular diseases or disorders, such as wet AMD, may be associated with elevated levels of PIGF. VEGF-B overexpression can be associated with breakdown of the blood-retinal barrier and retinal angiogenesis. Thus, inhibition of VEGF-A, VEGF-B, and PIGF may all contribute to the efficacy of aflibercept.

### Methods for Preparation of Vectors for Delivering Transgenes to Target Cells

In some embodiments, the rAAV particles are manufactured using any method known in the art. In some embodiments, the rAAV particles are manufactured using a baculovirus expression vector system in Sf9 cells. Sf9 cells are an insect cell culture cell line commonly used for recombinant protein production using baculovirus. In some embodiments, the rAAV particles are manufactured using two baculoviruses in Sf9 cells. In some embodiments, the rAAV particles are manufactured using two baculoviruses in Sf9 cells, wherein a first baculovirus encodes the genes for AAV2 Rep and AAV2.7m8 Cap proteins and a second baculovirus encodes an anti-VEGF agent. In some embodiments, the rAAV particles are manufactured using two baculoviruses in Sf9 cells, wherein a first baculovirus encodes the genes for AAV2 Rep and AAV2.7m8 Cap proteins and a second baculovirus encodes an aflibercept (e.g., human aflibercept) cDNA expression cassette. In some embodiments, the rAAV particles are manufactured using two baculoviruses in Sf9 cells, wherein a first baculovirus encodes the genes for AAV2 Rep and AAV2.7m8 Cap proteins and a second baculovirus comprises a nucleic acid encoding a polypeptide comprising an amino acid sequence with at least about 95% identity to the amino acid sequence of SEQ ID NO: 35 and flanked by AAV2 inverted terminal repeats (ITRs). In some embodiments, the polypeptide comprises the amino acid sequence of SEQ ID NO: 35. In some embodiments, the polypeptide is aflibercept.

### Doses

In some embodiments, the unit dose of rAAV particles is administered to one eye of the individual. In some embodiments, the one eye of the individual is the right eye or the left eye. In some embodiments, the one eye of the individual is the right eye. In some embodiments, the one eye of the individual is the left eye. In some embodiments, the methods provided herein further comprise administering a unit dose of rAAV particles to the contralateral eye of the individual. In some embodiments, the one eye of the individual is the right eye and the contralateral eye is the left eye. In some embodiments, the one eye of the individual is the left eye and the contralateral eye is the right eye.

In some embodiments, the administering the unit dose of rAAV particles to the contralateral eye is at least about 2 weeks (e.g., at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, or more) after administering the unit dose of rAAV particles to the one eye. In some embodiments, the administering the unit dose of rAAV particles to the contralateral eye is at least about 2 weeks after administering the unit dose of rAAV particles to the one eye and the unit dose of rAAV particles administered to the contralateral eye of the individual is higher (e.g., any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more, higher) than the unit dose of rAAV particles administered to the one eye of the individual.

In some embodiments, the administering the unit dose of rAAV particles to the contralateral eye of the individual is up to about 1 week, up to about 2 weeks, up to about 3 weeks, or up to about 4 weeks after administering the unit dose of rAAV particles to the one eye. In some embodiments, the administering the unit dose of rAAV particles to the contralateral eye of the individual is up to about 2 weeks (e.g., about 0 days, 1 day, 2 days, 3 days, 4 days, 5 days, 6 days, 7 days, 8 days, 9 days, 10 days, 11 days, 12 days, 13 days, or 14 days) after administering the unit dose of rAAV particles to the one eye. In some embodiments, the administering the unit dose of rAAV particles to the contralateral eye of the individual is up to about 2 weeks (e.g., about 0 days, 1 day, 2 days, 3 days, 4 days, 5 days, 6 days, 7 days, 8 days, 9 days, 10 days, 11 days, 12 days, 13 days, or 14 days) after administering the unit dose of rAAV particles to the one eye and the unit dose of rAAV particles administered to the contralateral eye of the individual is about the same as (e.g., less than 1% higher or lower, less than 5% higher or lower, less than 10% higher or lower, or less than 20% higher or lower), or lower (e.g., about 5%, about 10%, about 20%, about 30%, about 40%, about 50%, about 60%, about 70%, about 80%, or about 90% lower) than the unit dose of rAAV particles administered to the one eye of the individual. In some embodiments, the administering the unit dose of rAAV particles to the contralateral eye of the individual is up to about 2 weeks after administering the unit dose of rAAV particles to the one eye and the unit dose of rAAV particles administered to the contralateral eye of the individual is about the same (e.g., less than 1% higher or lower, less than 5% higher or lower, less than 10% higher or lower, or less than 20% higher or lower) as the unit dose of rAAV particles administered to the one eye of the individual. In some embodiments, the administering the unit dose of rAAV particles to the contralateral eye of the individual is up to about 2 weeks after administering the unit dose of rAAV particles to the one eye and the unit dose of rAAV particles administered to the contralateral eye of the individual is lower (e.g., about 5%, about 10%, about 20%, about 30%, about 40%, about 50%, about 60%, about 70%, about 80%, or about 90% lower) than the unit dose of rAAV particles administered to the one eye of the individual.

In some embodiments, the unit dose of rAAV particles is administered to one eye and/or to the contralateral eye of the individual. In some embodiments, the unit dose of rAAV particles is expressed as the number of vector genomes (vg). In some embodiments, the unit dose is about 6×10¹¹ vector genomes (vg) or less of the rAAV particles. In some embodiments, the unit dose is about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×10¹⁰ to about 6×10¹⁰, between about 6×10¹⁰ to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, or between about 5×10¹¹ to about 6×10¹¹ vg of the rAAV particles, including any value within these ranges, of the rAAV particles. In some embodiments, the unit dose is about 1×10¹¹ to about 5×10¹¹, between about 5×10¹¹ to about 1×10¹², between about 1×10¹² to about 5×10¹², between about 5×10¹² to about 1×10¹³, between about 1×10¹³ to about 5×10¹³, between about 5×10¹³ to about 1×10¹⁴ vg of the rAAV particles, including any value within these ranges, of the rAAV particles. In some embodiments, the unit dose is about 6×10¹⁰ vector genomes (vg) to about 2×10¹¹ vg of the rAAV particles. In some embodiments, the unit dose is about 6×10¹⁰ vg to about 2×10¹¹ vg, about 7×10¹⁰ vg to about 2×10¹¹ vg, about 8×10¹⁰ vg to about 2×10¹¹ vg, about 9×10¹⁰ vg to about 2×10¹¹ vg, about 10×10¹⁰ vg to about 2×10¹¹ vg, or about 1×10¹¹ vg to about 2×10¹¹ vg of the rAAV particles. In some embodiments, the unit dose is about 6×10¹⁰ vg to about 2×10¹¹ vg of the rAAV particles. In some embodiments, the unit dose is about 6×10¹⁰ vg to about 7×10¹⁰ vg, about 7×10¹⁰ vg to about 8×10¹⁰ vg, about 8×10¹⁰ vg to about 9×10¹⁰ vg, about 9×10¹⁰ vg to about 10×10¹⁰ vg, about 10×10¹⁰ vg to about 1×10¹¹ vg, or about 1×10¹¹ vg to about 2×10¹¹ vg of the rAAV particles. In some embodiments, the unit dose is about 6×10¹⁰ vg, about 7×10¹⁰ vg, about 8×10¹⁰ vg, about 9×10¹⁰ vg, about 10×10¹⁰ vg, about 1×10¹¹ vg, or about 2×10¹¹ vg of the rAAV particles. In some embodiments, the unit dose is about 6×10¹⁰ vg or about 2×10¹¹ vg of the rAAV particles. In some embodiments, the unit dose is about 6×10¹⁰ vg of the rAAV particles. In some embodiments, the unit dose is about 6×10¹⁰ vg, about 2×10¹¹ vg, or about 6×10¹¹ vg. In some embodiments, the unit dose is about 6×10¹⁰ vg. In some embodiments, the unit dose is about 2×10¹¹ vg. In some embodiments, the unit dose is about 6×10¹¹ vg.

In some embodiments, the unit dose of rAAV particles is administered to one eye and/or to the contralateral eye of the individual. In some embodiments, the unit dose is expressed as the number of vector genomes (vg) per eye (vg/eye). In some embodiments, the unit dose is about 6×10¹¹ vg/eye or less of the rAAV particles. In some embodiments, the unit dose is about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×10¹⁰ to about 6×10¹⁰, between about 6×10¹⁰ to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, or between about 5×10¹¹ to about 6×10¹¹ vg/eye of the rAAV particles, including any value within these ranges, of the rAAV particles. In some embodiments, the unit dose is about 1×10¹¹ to about 5×10¹¹, between about 5×10¹¹ to about 1×10¹², between about 1×10¹² to about 5×10¹², between about 5×10¹² to about 1×10¹³, between about 1×10¹³ to about 5×10¹³, between about 5×10¹³ to about 1×10¹⁴ vg of the rAAV particles, including any value within these ranges, of the rAAV particles. In some embodiments, the unit dose is about 6×10¹⁰ vg/eye to about 2×10¹¹ vg/eye of the rAAV particles. In some embodiments, the unit dose is about 6×10¹⁰ vg/eye to about 2×10¹¹ vg/eye, about 7×10¹⁰ vg/eye to about 2×10¹¹ vg/eye, about 8×10¹⁰ vg/eye to about 2×10¹¹ vg/eye, about 9×10¹⁰ vg/eye to about 2×10¹¹ vg/eye, about 10×10¹⁰ vg/eye to about 2×10¹¹ vg/eye, or about 1×10¹¹ vg/eye to about 2×10¹¹ vg/eye of the rAAV particles. In some embodiments, the unit dose is about 6×10¹⁰ vg/eye to about 2×10¹¹ vg/eye of the rAAV particles. In some embodiments, the unit dose is about 6×10¹⁰ vg/eye to about 7×10¹⁰ vg/eye, about 7×10¹⁰ vg/eye to about 8×10¹⁰ vg/eye, about 8×10¹⁰ vg/eye to about 9×10¹⁰ vg/eye, about 9×10¹⁰ vg/eye to about 10×10¹⁰ vg/eye, about 10×10¹⁰ vg/eye to about 1×10¹¹vg/eye, or about 1×10¹¹ vg/eye to about 2×10¹¹ vg/eye of the rAAV particles. In some embodiments, the unit dose is about 6×10¹⁰ vg/eye, about 7×10¹⁰ vg/eye, about 8×10¹⁰ vg/eye, about 9×10¹⁰ vg/eye, about 10×10¹⁰ vg/eye, about 1×10¹¹ vg/eye, or about 2×10¹¹ vg/eye of the rAAV particles. In some embodiments, the unit dose is about 6×10¹⁰ vg/eye or about 2×10¹¹ vg/eye of the rAAV particles. In some embodiments, the unit dose is about 6×10¹⁰ vg/eye of the rAAV particles. In some embodiments, the unit dose is about 6×10¹⁰ vg/eye, about 2×10¹¹ vg/eye, or about 6×10¹¹ vg/eye. In some embodiments, the unit dose is about 6×10¹⁰ vg/eye. In some embodiments, the unit dose is about 2×10¹¹ vg/eye. In some embodiments, the unit dose is about 6×10¹¹ vg/eye.

In some embodiments, the unit dose of rAAV particles is administered to one eye and/or to the contralateral eye of the individual. In some embodiments, E is a shorthand for base 10 for exponentiation, and xEy refers to x multiplied by base 10 to the y power/exponent. In some embodiments, the unit dose is expressed as the number of vector genomes (vg). In some embodiments, the unit dose is about 6E¹¹ vector genomes (vg) or less of the rAAV particles. In some embodiments, the unit dose is about 1E¹⁰ to about 2E¹⁰, between about 2E¹⁰ to about 3E¹⁰, between about 3E¹⁰ to about 4E¹⁰, between about 4E¹⁰ to about 5E¹⁰, between about 5E¹⁰ to about 6E¹⁰, between about 6E¹⁰ to about 7E¹⁰, between about 7E¹⁰ to about 8E¹⁰, between about 8E¹⁰ to about 9E¹⁰, between about 9E¹⁰ to about 10E¹⁰, between about 1E¹¹ to about 2E¹¹, between about 2E¹¹ to about 3E¹¹, between about 3E¹¹ to about 4E¹¹, between about 4E¹¹ to about 5E¹¹, or between about 5E¹¹ to about 6E¹¹ vg of the rAAV particles, including any value within these ranges, of the rAAV particles. In some embodiments, the unit dose is about 6E¹⁰ vector genomes (vg) to about 2E¹¹ vg of the rAAV particles. In some embodiments, the unit dose is about 6E¹⁰ vg to about 2E¹¹ vg, about 7E¹⁰ vg to about 2E¹¹ vg, about 8E¹⁰ vg to about 2E¹¹ vg, about 9E¹⁰ vg to about 2E¹¹ vg, about 10E¹⁰ vg to about 2E¹¹ vg, or about 1E¹¹ vg to about 2E¹¹ vg of the rAAV particles. In some embodiments, the unit dose is about 6E¹⁰ vg to about 2E¹¹ vg of the rAAV particles. In some embodiments, the unit dose is about 6E¹⁰ vg to about 7E¹⁰ vg, about 7E¹⁰ vg to about 8E¹⁰ vg, about 8E¹⁰ vg to about 9E¹⁰ vg, about 9E¹⁰ vg to about 10E¹⁰ vg, about 10E¹⁰ vg to about 1E¹¹ vg, or about 1E¹¹ vg to about 2E¹¹ vg of the rAAV particles. In some embodiments, the unit dose is about 6E¹⁰ vg, about 7E¹⁰ vg, about 8E¹⁰ vg, about 9E¹⁰ vg, about 10E¹⁰ vg, about 1E¹¹ vg, or about 2E¹¹ vg of the rAAV particles. In some embodiments, the unit dose is about 6E¹⁰ vg or about 2E¹¹ vg of the rAAV particles. In some embodiments, the unit dose is about 6E¹⁰ vg of the rAAV particles. In some embodiments, the unit dose is about 6E¹⁰ vg, about 2E¹¹ vg, or about 6E¹¹ vg. In some embodiments, the unit dose is about 6E¹⁰ vg. In some embodiments, the unit dose is about 2E¹¹ vg. In some embodiments, the unit dose is about 6E¹ vg.

In some embodiments, the unit dose of rAAV particles is administered to one eye and/or to the contralateral eye of the individual. In some embodiments, the unit dose is expressed as the number of vector genomes (vg) per eye (vg/eye). In some embodiments, the unit dose is about 6E¹¹ vg/eye or less of the rAAV particles. In some embodiments, the unit dose is about 1E¹⁰ to about 2E¹⁰, between about 2E¹⁰ to about 3E¹⁰, between about 3E¹⁰ to about 4E¹⁰, between about 4E¹⁰ to about 5E¹⁰, between about 5E¹⁰ to about 6E¹⁰, between about 6E¹⁰ to about 7E¹⁰, between about 7E¹⁰ to about 8E¹⁰, between about 8E¹⁰ to about 9E¹⁰, between about 9E¹⁰ to about 10E¹⁰, between about 1E¹¹ to about 2E¹¹, between about 2E¹¹ to about 3E¹¹, between about 3E¹¹ to about 4E¹¹, between about 4E¹¹ to about 5E¹¹, or between about 5E¹¹ to about 6E¹¹ vg/eye of the rAAV particles, including any value within these ranges, of the rAAV particles. In some embodiments, the unit dose is about 6E¹⁰ vg/eye to about 2E¹¹ vg/eye of the rAAV particles. In some embodiments, the unit dose is about 6E¹⁰ vg/eye to about 2E¹¹ vg/eye, about 7E¹⁰ vg/eye to about 2E¹¹ vg/eye, about 8E¹⁰ vg/eye to about 2E¹¹ vg/eye, about 9E¹⁰ vg/eye to about 2E¹¹ vg/eye, about 10E¹⁰ vg/eye to about 2E¹¹ vg/eye, or about 1E¹¹ vg/eye to about 2E¹¹ vg/eye of the rAAV particles. In some embodiments, the unit dose is about 6E¹⁰ vg/eye to about 2E¹¹ vg/eye of the rAAV particles. In some embodiments, the unit dose is about 6E¹⁰ vg/eye to about 7E¹⁰ vg/eye, about 7E¹⁰ vg/eye to about 8E¹⁰ vg/eye, about 8E¹⁰ vg/eye to about 9E¹⁰ vg/eye, about 9E¹⁰ vg/eye to about 10E¹ vg/eye, about 10E¹ vg/eye to about 1E¹¹ vg/eye, or about 1E¹¹ vg/eye to about 2E¹¹ vg/eye of the rAAV particles. In some embodiments, the unit dose is about 6E¹⁰ vg/eye, about 7E¹⁰ vg/eye, about 8E¹⁰ vg/eye, about 9E¹⁰ vg/eye, about 10E¹⁰ vg/eye, about 1E¹¹ vg/eye, or about 2E¹¹ vg/eye of the rAAV particles. In some embodiments, the unit dose is about 6E¹⁰ vg/eye or about 2E¹¹ vg/eye of the rAAV particles. In some embodiments, the unit dose is about 6E¹⁰ vg/eye of the rAAV particles. In some embodiments, the unit dose is about 6E¹⁰ vg/eye, about 2E¹¹ vg/eye, or about 6E¹¹ vg/eye. In some embodiments, the unit dose is about 6E¹⁰ vg/eye. In some embodiments, the unit dose is about 2E¹¹ vg/eye. In some embodiments, the unit dose is about 6E¹¹ vg/eye.

In some embodiments, the unit dose of rAAV particles is administered to one eye and/or to the contralateral eye of the individual. In some embodiments, the unit dose of rAAV particles is a unit dose sufficient to cause expression of the therapeutic protein (e.g., an anti-VEGF agent such as aflibercept) in the vitreous fluid. In some embodiments, the unit dose of rAAV particles is a unit dose sufficient to achieve a concentration of the therapeutic protein (e.g., an anti-VEGF agent such as aflibercept) at about any one of 3, 3.5, 4, 4.5, 5, 5.5, 6, 6.5, 7, 7.5, 8, 8.5, 9, 9.5, 10 μg/ml, or more, including any range in between these values, in the vitreous fluid. In some embodiments, the unit dose of rAAV particles is a unit dose sufficient to cause expression of aflibercept in the vitreous fluid. In some embodiments, the unit dose of rAAV particles is a unit dose sufficient to achieve a concentration of aflibercept at about any one of 3, 3.5, 4, 4.5, 5, 5.5, 6, 6.5, 7, 7.5, 8, 8.5, 9, 9.5, 10 μg/ml, or more, including any range in between these values, in the vitreous fluid.

In some embodiments, the unit dose of rAAV particles administered to the one eye and/or to the contralateral eye of the individual is a unit dose sufficient to cause expression of the therapeutic protein (e.g., an anti-VEGF agent such as aflibercept) in the aqueous fluid. In some embodiments, the unit dose of rAAV particles is a unit dose sufficient to achieve a concentration of the therapeutic protein (e.g., an anti-VEGF agent such as aflibercept) of at least about 0.5, 0.55, 0.6, 0.65, 0.7, 0.75, 0.8, 0.85, 0.9, 0.95, 1.0 μg/ml, or more, including any range in between these values, in the aqueous fluid. In some embodiments, the unit dose of rAAV particles is a unit dose sufficient to cause expression of aflibercept in the aqueous fluid. In some embodiments, the unit dose of rAAV particles is a unit dose sufficient to achieve a concentration of aflibercept of at least about 0.5, 0.55, 0.6, 0.65, 0.7, 0.75, 0.8, 0.85, 0.9, 0.95, 1.0 μg/ml, or more, including any range in between these values, in the aqueous fluid.

In some embodiments, the unit dose of rAAV particles is administered to one eye and/or to the contralateral eye of the individual. In some embodiments, the unit dose of rAAV particles is a unit dose sufficient to cause expression of the therapeutic protein (e.g., an anti-VEGF agent such as aflibercept) in the retina. In some embodiments, the unit dose of rAAV particles is a unit dose sufficient to achieve a concentration of the therapeutic protein (e.g., an anti-VEGF agent such as aflibercept) of at least about 3, 3.5, 4, 4.5, 5, 5.5, 6, 6.5, 7, 7.5, 8, 8.5, 9, 9.5, 10 μg/g, or more, including any range in between these values, in the retina. In some embodiments, the unit dose of rAAV particles is a unit dose sufficient to cause expression of aflibercept in the retina. In some embodiments, the unit dose of rAAV particles is a unit dose sufficient to achieve a concentration of aflibercept of at least about 3, 3.5, 4, 4.5, 5, 5.5, 6, 6.5, 7, 7.5, 8, 8.5, 9, 9.5, 10 μg/g, or more, including any range in between these values, in the retina.

In some embodiments, the unit dose of rAAV particles is administered to one eye and/or to the contralateral eye of the individual. In some embodiments, the unit dose of rAAV particles is a unit dose sufficient to cause expression of the therapeutic protein (e.g., an anti-VEGF agent such as aflibercept) in the choroid. In some embodiments, the unit dose of rAAV particles is a unit dose sufficient to achieve a concentration of the therapeutic protein (e.g., an anti-VEGF agent such as aflibercept) at about any one of 3, 3.5, 4, 4.5, 5, 5.5, 6, 6.5, 7, 7.5, 8, 8.5, 9, 9.5, 10 μg/g, or more, including any range in between these values, in the choroid. In some embodiments, the unit dose of rAAV particles is a unit dose sufficient to cause expression of aflibercept in the choroid. In some embodiments, the unit dose of rAAV particles is a unit dose sufficient to achieve a concentration of aflibercept at about any one of 3, 3.5, 4, 4.5, 5, 5.5, 6, 6.5, 7, 7.5, 8, 8.5, 9, 9.5, 10 μg/g, or more, including any range in between these values, in the choroid.

In some embodiments, the unit dose of rAAV particles is administered to one eye and/or to the contralateral eye of the individual. In some embodiments, the unit dose of rAAV particles is a therapeutically effective dose.

In some embodiments, the unit dose of rAAV particles is a therapeutically effective dose if the unit dose is sufficient to cause maintenance or an improvement of visual acuity compared to the visual acuity prior to administration of the unit dose of rAAV particles. In some embodiments, the unit dose of rAAV particles is a therapeutically effective dose if the unit dose is sufficient to cause an improvement of visual acuity compared to the visual acuity prior to administration of the unit dose of rAAV particles. In some embodiments, the unit dose of rAAV particles is a therapeutically effective dose if the unit dose is sufficient to cause an improvement of visual acuity of more than any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more compared to the visual acuity prior to administration of the unit dose of rAAV particles. In some embodiments, visual acuity is best corrected visual acuity (BCVA). In some embodiments, the unit dose of rAAV particles is a therapeutically effective dose if the unit dose is sufficient to cause an improvement of BCVA compared to the BCVA prior to administration of the unit dose of rAAV particles. In some embodiments, BCVA is expressed as an ETDRS score, which corresponds to the number of letters correctly read (Vitale et al., (2016) JAMA Opthalmol 134(9):1041:1047). In some embodiments, the unit dose of rAAV particles is a therapeutically effective dose if the unit dose is sufficient to cause an improvement of BCVA of at least 15 ETDRS letters (Vitale et al., (2016) JAMA Opthalmol 134(9):1041:1047) (e.g., at least about 15, at least about 20, at least about 30, at least about 40, at least about 50, at least about 60, or about 70 letters) compared to the BCVA prior to administration of the unit dose of rAAV particles. In some embodiments, the unit dose of rAAV particles is a therapeutically effective dose if the unit dose is sufficient to cause maintenance of BCVA, wherein the individual loses fewer than 15 ETDRS letters (Vitale et al., (2016) JAMA Opthalmol 134(9):1041:1047) (e.g., 15 or less, 14 or less, 13 or less, 12 or less, 11 or less, 10 or less, 9 or less, 8 or less, 7 or less, 6 or less, 5 or less, 4 or less, 3 or less, 2 or less, 1, or 0 letters) compared to the BCVA prior to administration of the unit dose of rAAV particles.

In some embodiments, the unit dose of rAAV particles is a therapeutically effective dose if, after administration of the unit dose of rAAV particles, the individual is determined to have maintenance of vision. In some embodiments, the unit dose of rAAV particles is a therapeutically effective dose if, after administration of the unit dose of rAAV particles, the individual is determined have an improvement of vision.

In some embodiments, the unit dose of rAAV particles is a therapeutically effective dose if, after administration of the unit dose of rAAV particles, the individual requires less than one rescue therapy treatment (e.g., aflibercept injection) about any of every 4 weeks, every 5 weeks, every 6 weeks, every 7 weeks, every 8 weeks, every 9 weeks, every 10 weeks, or more after administration of the unit dose of rAAV particles in the one eye and/or the contralateral eye. In some embodiments, the unit dose of rAAV particles is a therapeutically effective dose if, after administration of the unit dose of rAAV particles, the individual does not require any rescue therapy treatment (e.g., aflibercept injection) for at least about any of 1 week, at least 2 weeks, at least 3 weeks, at least 4 weeks, at least 5 weeks, at least 6 weeks, at least 7 weeks, at least 8 weeks, at least 9 weeks, at least 10 weeks, at least 15 weeks, at least 20 weeks, at least 30 weeks, at least 40 weeks, at least 50 weeks, at least 60 weeks, at least 70 weeks, at least 80 weeks, at least 90 weeks, at least 100 weeks, at least 110 weeks, or more.

In some embodiments, the unit dose of rAAV particles is a therapeutically effective dose if, after administration of the unit dose of rAAV particles, the individual is determined to have a resolution of pigment epithelial detachment (PED) compared to PED prior to administration of the unit dose of rAAV particles in the one eye and/or the contralateral eye.

In some embodiments, the unit dose of rAAV particles is a therapeutically effective dose if, after administration of the unit dose of rAAV particles, CNV lesions shrink compared to CNV lesions present prior to administration of the unit dose of rAAV particles in the one eye and/or the contralateral eye. In some embodiments, the unit dose of rAAV particles is a therapeutically effective dose if, after administration of the unit dose of rAAV particles, CNV lesions shrink by more than any of about 5%, 10%, 20%, 30%, 40%, 50%, 60%, 70%, 80%, 90%, or 100% compared to CNV lesions present prior to administration of the unit dose of rAAV particles in the one eye and/or the contralateral eye. In some embodiments, the unit dose of rAAV particles is a therapeutically effective dose if, after administration of the unit dose of rAAV particles, CNV lesions do not grow compared to CNV lesions present prior to administration of the unit dose of rAAV particles in the one eye and/or the contralateral eye. In some embodiments, the unit dose of rAAV particles is a therapeutically effective dose if, after administration of the unit dose of rAAV particles, CNV lesions do not grow by more than about any of about 1%, 2%, 3%, 4%, 5%, 6%, 7%, 8%, 9%, 10%, 15%, or 20% compared to CNV lesions present prior to administration of the unit dose of rAAV particles in the one eye and/or the contralateral eye.

In some embodiments, the unit dose of rAAV particles is a therapeutically effective dose if, after administration of the unit dose of rAAV particles, the individual is determined to have an improvement in anatomical features of the one eye and/or the contralateral eye compared to the anatomical features prior to administration of the unit dose of rAAV particles. In some embodiments, the unit dose of rAAV particles is a therapeutically effective dose if, after administration of the unit dose of rAAV particles, the individual is determined to have a stabilization and/or maintenance of anatomical features of the one eye and/or the contralateral eye compared to the anatomical features prior to administration of the unit dose of rAAV particles.

In some embodiments, the unit dose of rAAV particles is therapeutically effective if administration of the dose to the one eye and/or the contralateral eye of the individual reduces, stops, or prevents at least one symptom of the ocular disease or disorder. In some embodiments, such symptoms include, but are not limited to, e.g., visual distortions (such as impaired color vision, blurred vision, deterioration of central vision), vision loss, change of degree of iris rubeosis, change in intraocular pressure (IOP), change in number of additional anti-glaucoma interventions (anti-glaucomatous medications, surgeries, etc.), and/or change in gonioscopy of the anterior chamber angle.

In some embodiments, the unit dose of rAAV particles administered to the one eye and/or to the contralateral eye of the individual is a therapeutically effective dose if administration of the unit dose to the one eye and/or to the contralateral eye of the individual results in the maintenance, partial resolution, or complete resolution of one or more clinical features of the ocular disease (e.g., glaucoma, such as neovascular glaucoma). For example, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular disease (e.g., glaucoma, such as neovascular glaucoma) as measured by any method known in the art. In some embodiments, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular disease as assessed by best corrected visual acuity (BCVA) (e.g., based on an ETDRS score; Vitale et al., (2016) JAMA Opthalmol 134(9):1041:1047), the number of rescue therapy treatments (e.g., aflibercept injections) required by the individual after administration of the unit dose of rAAV particles in the one eye and/or the contralateral eye, the resolution of pigment epithelial detachment (PED), choroidal neovascularization (CNV) lesion growth, iris rubeosis, anatomical features based on any methods known in the art (e.g., SD-OCT, OCT, fluorescein angiography, digital color fundus photography, etc.). In some embodiments, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular disease as assessed by ophthalmologic examination, intraocular pressure (e.g., using a Goldmann applanation tonometer or Tono-pen), indirect ophthalmoscopy, examination of the one eye and/or the contralateral eye and adnexa, eyelid and/or pupil responsiveness, belpharoptosis, abnormal pupil shape, unequal pupils, abnormal reaction to light, afferent pupillary defects, slit-lamp examination (including of the eyelids, conjunctiva, cornea, lens, iris, and anterior chamber), posterior segment abnormalities of the vitreous, optic nerve, peripheral retina, and retinal vasculature, SD-OCT, fluorescein angiography, digital color fundus photography (including images of the retina, optic disc, and/or macula), aqueous humor sampling, vitreous humor sampling, OCT-angiography (OCT-A), refraction, and visual acuity (BCVA). In some embodiments, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular disease (e.g. glaucoma or neovascular glaucoma) as assessed by gonioscopy of the anterior chamber angle. Gonioscopy is typically performed during the eye exam to evaluate the internal drainage system of the eye, also referred to as the anterior chamber angle. The “angle” is where the cornea and the iris meet. This is the location where fluid inside the eye (aqueous humor) drains out of the eye and into the venous system.

In some embodiments, the unit dose of rAAV particles administered to the one eye of the individual is the same as the unit dose of rAAV particles administered to the contralateral eye of the individual. In some embodiments, the unit dose of rAAV particles administered to the one eye of the individual is different from the unit dose of rAAV particles administered to the contralateral eye of the individual. In some embodiments, the unit dose of rAAV particles administered to the one eye of the individual is higher, e.g., more than any of about 10%, about 20%, about 30%, about 40%, about 50%, about 60%, about 70%, about 80%, about 90%, about 100%, about 125%, about 150%, about 175%, about 200%, about 225%, about 250%, about 275%, about 300% or more, than the unit dose of rAAV particles administered to the contralateral eye of the individual. In some embodiments, the unit dose of rAAV particles administered to the contralateral eye of the individual is higher, e.g., more than any of about 10%, about 20%, about 30%, about 40%, about 50%, about 60%, about 70%, about 80%, about 90%, about 100%, about 125%, about 150%, about 175%, about 200%, about 225%, about 250%, about 275%, about 300% or more, than the unit dose of rAAV particles administered to the one eye of the individual. In some embodiments, the unit dose of rAAV particles is expressed as the number of vector genomes (vg) per eye (vg/eye). In some embodiments, the unit dose of rAAV particles is about 6×10¹¹ vg/eye or less of the rAAV particles. In some embodiments, the unit dose of rAAV particles is about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×10¹⁰ to about 6×10¹⁰, between about 6×10¹⁰ to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, or between about 5×10¹¹ to about 6×10¹¹ vg/eye of the rAAV particles, including any value within these ranges, of the rAAV particles. In some embodiments, the unit dose of rAAV particles is about 6×10¹⁰ vg/eye to about 2×10¹¹ vg/eye of the rAAV particles. In some embodiments, the unit dose of rAAV particles is about 6×10¹⁰ vg/eye to about 2×10¹¹ vg/eye, about 7×10¹⁰ vg/eye to about 2×10¹¹ vg/eye, about 8×10¹⁰ vg/eye to about 2×10¹¹ vg/eye, about 9×10¹⁰ vg/eye to about 2×10¹¹ vg/eye, about 10×10¹⁰ vg/eye to about 2×10¹¹vg/eye, or about 1×10¹¹ vg/eye to about 2×10¹¹ vg/eye of the rAAV particles. In some embodiments, the unit dose of rAAV particles is about 6×10¹⁰ vg/eye to about 2×10¹¹ vg/eye of the rAAV particles. In some embodiments, the unit dose of rAAV particles is about 6×10¹⁰ vg/eye to about 7×10¹ vg/eye, about 7×10¹⁰ vg/eye to about 8×10¹⁰ vg/eye, about 8×10¹⁰ vg/eye to about 9×10¹⁰ vg/eye, about 9×10¹⁰ vg/eye to about 10×10¹⁰ vg/eye, about 10×10¹⁰ vg/eye to about 1×10¹¹ vg/eye, or about 1×10¹¹ vg/eye to about 2×10¹¹ vg/eye of the rAAV particles. In some embodiments, the unit dose of rAAV particles is about 6×10¹⁰ vg/eye, about 7×10¹⁰ vg/eye, about 8×10¹⁰ vg/eye, about 9×10¹⁰ vg/eye, about 10×10¹⁰ vg/eye, about 1×10¹¹ vg/eye, or about 2×10¹¹ vg/eye of the rAAV particles. In some embodiments, the unit dose of rAAV particles is about 6×10¹⁰ vg/eye or about 2×10¹¹ vg/eye of the rAAV particles. In some embodiments, the unit dose of rAAV particles is about 6×10¹⁰ vg/eye of the rAAV particles. In some embodiments, the unit dose of rAAV particles is about 6×10¹⁰ vg/eye, about 2×10¹¹ vg/eye, or about 6×10¹¹ vg/eye. In some embodiments, the unit dose of rAAV particles is about 6×10¹⁰ vg/eye. In some embodiments, the unit dose of rAAV particles is about 2×10¹¹ vg/eye. In some embodiments, the unit dose of rAAV particles is about 6×10¹¹ vg/eye.

In some embodiments, the unit dose of rAAV particles administered to the one eye of the individual and the unit dose of rAAV particles administered to the contralateral eye of the individual are administered at the same time. In some embodiments, the unit dose of rAAV particles administered to the one eye of the individual and the unit dose of rAAV particles administered to the contralateral eye of the individual are administered at different times. In some embodiments, the unit dose administered to the contralateral eye is administered any of at least about 1 hour, at least about 2 hours, at least about 4 hours, at least about 8 hours, at least about 12 hours, at least about 24 hours, at least about 1 day, at least about 2 days, at least about 3 days, at least about 4 days, at least about 5 days, at least about 6 days, at least about 7 days, at least about 1 week, at least about 2 weeks, at least about 3 weeks, at least about 4 weeks, or more after administering of the unit dose to the one eye. In some embodiments, the unit dose administered to the contralateral eye is administered at least about 2 weeks after administering of the unit dose to the one eye.

In some embodiments, a single unit dose of rAAV particles is administered to the one eye and/or the contralateral eye of the individual. In some embodiments, the single unit dose of rAAV particles administered to the one eye and/or to the contralateral eye is a therapeutically effective dose. In some embodiments, more than one dose of rAAV particles (e.g., more than any of about 2, 3, 4, 5, or more unit doses) are administered to the one eye and/or the contralateral eye of the individual. In some embodiments, the more than one doses of rAAV particles administered to the one eye and/or to the contralateral are therapeutically effective doses.

In some embodiments, an anti-VEGF treatment, e.g., an IVT injection with an anti-VEGF agent such as aflibercept, is administered to the one eye and/or to the contralateral eye administered the rAAV particles at least about one week, e.g. at least about 7 days, prior to administration of the unit dose of rAAV particles (e.g., a unit dose of between about 2×10¹¹ vg/eye to about 6×10¹¹ vg/eye of rAAV particles). In some embodiments, an anti-VEGF treatment, e.g., an IVT injection with an anti-VEGF agent such as aflibercept, is administered to an eye on about Day 1 and the unit dose of rAAV particles, e.g., a unit dose of between about 2×10¹¹ vg/eye to about 6×10¹¹ vg/eye of rAAV particles, is administered to the eye on about Day 8. In some embodiments, the unit dose of rAAV particles is about 2×10¹¹ vg/eye or about 6×10¹¹ vg/eye of rAAV particles. In some embodiments, the ocular disease is diabetic macular edema.

### Pharmaceutical Formulations

In some embodiments, the unit dose of rAAV particles is in a pharmaceutical formulation. In some embodiments, the pharmaceutical formulation comprises the rAAV particles, one or more osmotic or ionic strength agents, one or more buffering agents, one or more surfactants, and one or more solvents. In some embodiments, the osmotic or ionic strength agent is sodium chloride. In some embodiments, the one or more buffering agents are sodium phosphate monobasic and/or sodium phosphate dibasic. In some embodiments, the surfactant is Poloxamer 188. In some embodiments, the solvent is water. In some embodiments, the pharmaceutical formulation comprises the rAAV particles, sodium chloride, sodium phosphate monobasic, sodium phosphate dibasic, and a surfactant.

In some embodiments, the pharmaceutical formulation comprises about 1×10¹⁰ vg/mL to about 1×10¹³ vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5. In some embodiments, the pharmaceutical formulation comprises about 6×10¹¹ vg/mL to about 6×10¹² vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5. In some embodiments, the pharmaceutical formulation comprises about 6×10¹¹ vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5. In some embodiments, the pharmaceutical formulation comprises about 6×10¹² vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5.

In some embodiments, the rAAV particles in the pharmaceutical formulation are present at a concentration of about 1×10¹⁰ vg/ml to about 1×10¹³ vg/ml. In some embodiments, the rAAV particles in the pharmaceutical formulation are present at a concentration of about 1×10⁰⁹ vg/ml to about 6×10¹⁴ vg/ml. In certain embodiments, the rAAV particles in the pharmaceutical formulation are present at a concentration of about 1×10° ⁹ vg/ml to about 2×10⁰9 vg/ml, about 2×10⁰9 vg/ml to about 3×10⁰⁹, about 3×10⁰9 vg/ml to about 4×10⁰⁹, about 4×10⁰9 vg/ml to about 5×10⁰⁹, about 5×10⁰⁹ vg/ml to about 6×10⁰⁹, about 6×10⁰9 vg/ml to about 7×10⁰⁹, about 7×10⁰⁹ vg/ml to about 8×10⁰⁹, about 8×10⁰9 vg/ml to about 9×10⁰⁹, about 9×10⁰9 vg/ml to about 10×10⁰⁹, about 10×1⁰° 9 vg/ml to about 1×10¹⁰, about 1×10¹⁰ vg/ml to about 2×10¹⁰, about 2×10¹⁰ vg/ml to about 3×10¹⁰, about 3×10¹⁰ vg/ml to about 4×10¹⁰, about 4×10¹⁰ vg/ml to about 5×10¹⁰, about 5×10¹⁰ vg/ml to about 6×10¹⁰, about 6×10¹⁰ vg/ml to about 7×10¹⁰, about 7×10¹⁰ vg/ml to about 8×10¹⁰, about 8×10¹⁰ vg/ml to about 9×10¹⁰, about 9×10¹⁰ vg/ml to about 10×10¹⁰, about loxl¹⁰1° vg/ml to about 1×10¹¹, about 1×10¹¹ vg/ml to about 2×10¹¹, about 2×10¹¹ vg/ml to about 3×10¹¹, about 3×10¹¹ vg/ml to about 4×10¹¹, about 4×10¹¹ vg/ml to about 5×10¹¹, about 5×10¹¹ vg/ml to about 6×10¹¹, about 6×10¹¹ vg/ml to about 7×10¹¹, about 7×10¹¹ vg/ml to about 8×10¹¹, about 8×10¹¹ vg/ml to about 9×10¹¹, about 9×10¹¹ vg/ml to about 10×10¹¹, about 1×10¹² vg/ml to about 2×10¹², about 2×10¹² vg/ml to about 3×10¹², about 3×10¹² vg/ml to about 4×10¹², about 4×10¹² vg/ml to about 5×10¹², about 5×10¹² vg/ml to about 6×10¹², about 6×10¹² vg/ml to about 7×10¹², about 7×10¹² vg/ml to about 8×10¹², about 8×10¹² vg/ml to about 9×10¹², about 9×10¹² vg/ml to about 10×10¹², about 1×10¹³ vg/ml to about 2×10¹³, about 2×10¹³ vg/ml to about 3×10¹³, about 3×10¹³ vg/ml to about 4×10¹³, about 4×10¹³ vg/ml to about 5×10¹³, about 5×10¹³ vg/ml to about 6×10¹³, about 6×10¹³ vg/ml to about 7×10¹³, about 7×10¹³ vg/ml to about 8×10¹³, about 8×10¹³ vg/ml to about 9×10¹³, about 9×10¹³ vg/ml to about 10×10¹³, about 1×10⁴ vg/ml to about 2×10¹⁴, about 2×10¹⁴ vg/ml to about 3×10¹⁴, about 3×10¹⁴ vg/ml to about 4×10¹⁴, about 4×10¹⁴ vg/ml to about 5×10¹⁴, or about 5×10¹⁴ vg/ml to about 6×10¹⁴ vg/mL. In some embodiments, the pharmaceutical formulation comprises about 6×10¹¹ vg/mL to about 6×10¹² vg/mL of rAAV particles. In some embodiments, the pharmaceutical formulation comprises about 6×10¹² vg/mL of rAAV particles. In some embodiments, the pharmaceutical formulation comprises about 6×10¹¹ vg/mL of rAAV particles.

In some embodiments, the sodium chloride in the pharmaceutical formulation is present at a concentration of about 150 mM to about 200 mM. In certain embodiments, the sodium chloride in the pharmaceutical formulation is present at a concentration of about 150 mM, about 160 mM, about 170 mM, about 180 mM, about 190 mM, or about 200 mM. In certain embodiments, the sodium chloride in the pharmaceutical formulation is present at a concentration of about 180 mM.

In some embodiments, the sodium phosphate monobasic is present in the pharmaceutical formulation at a concentration of about 1 mM to about 10 mM. In some embodiments, the sodium phosphate monobasic is present in the pharmaceutical formulation at a concentration of any of about 1 mM, about 2 mM, about 3 mM, about 4 mM, about 5 mM, about 6 mM, about 7 mM, about 8 mM, about 9 mM, or about 10 mM. In certain embodiments, the sodium phosphate monobasic is present in the pharmaceutical formulation at a concentration of about 5 mM.

In some embodiments, the sodium phosphate dibasic is present in the pharmaceutical formulation at a concentration of about 1 mM to about 10 mM. In some embodiments, the sodium phosphate dibasic is present in the pharmaceutical formulation at a concentration of any of about 1 mM, about 2 mM, about 3 mM, about 4 mM, about 5 mM, about 6 mM, about 7 mM, about 8 mM, about 9 mM, or about 10 mM. In certain embodiments, the sodium phosphate dibasic is present in the pharmaceutical formulation at a concentration of about 5 mM.

In some embodiments, the Poloxamer 188 is present in the pharmaceutical formulation at a concentration of about 0.0005% (w/v) to about 0.005% (w/v). In some embodiments, the Poloxamer 188 is present in the pharmaceutical formulation at a concentration of any of about 0.0005% (w/v), about 0.0006% (w/v), about 0.0007% (w/v), about 0.0008% (w/v), about 0.0009% (w/v), about 0.001% (w/v), about 0.002% (w/v), about 0.003% (w/v), about 0.004% (w/v), or about 0.005% (w/v). In certain embodiments, the Poloxamer 188 is present in the pharmaceutical formulation at a concentration of about 0.001% (w/v).

In some embodiments, the pharmaceutical formulation has a pH of about 7.0 to about 7.5. In some embodiments, the pharmaceutical formulation has a pH of about 7.0, about 7.1, about 7.2, about 7.3, about 7.4, or about 7.5. In certain embodiments, the pharmaceutical formulation has a pH of about 7.3. In some embodiments, hydrochloric acid and sodium hydroxide are used to adjust the pH of the pharmaceutical formulation.

In some embodiments, the pharmaceutical formulation comprises about 6×10¹² vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3. In some embodiments, the pharmaceutical formulation comprises about 6×10¹¹ vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3.

In some embodiments, the pharmaceutical formulations are suitable for administration to the one eye and/or the contralateral eye of the individual, e.g., a human patient, via intravitreal (IVT) injection to achieve a desired therapeutic or prophylactic effect. In some embodiments, the pharmaceutical formulation is supplied as a reconstituted homogenous solution. In some embodiments, the solution is a suspension. In some embodiments, the pharmaceutical formulation is supplied as a frozen suspension, and is thawed prior to administration to the one eye and/or the contralateral eye of the individual. In some embodiments, the solution is isotonic.

In other embodiments, the pharmaceutical composition comprising e.g., an AAV2.7m8 vector that comprises a nucleic acid sequence encoding the anti-VEGF agent (e.g., aflibercept or a functional fragment or variant thereof), is supplied in a lyophilized form, and is reconstituted prior to administration to the one eye and/or the contralateral eye of the individual. In some embodiments, the methods provided herein further comprise the steps of reconstituting, dissolving, or solubilizing a lyophilized pharmaceutical composition comprising rAAV (e.g., AAV2.7m8) and encoding the anti-VEGF agent (e.g., aflibercept or a functional fragment or variant thereof) in a buffer prior to administration to the subject. In some embodiments, such lyophilized pharmaceutical composition comprises one or more of the following: a cryoprotectant, a surfactant, a salt, a stabilizer, or any combination thereof.

In some embodiments, the pharmaceutical formulation is a homogenous solution. In some embodiments, the homogenous solution is supplied in a pre-filled syringe. In some embodiments, the pharmaceutical formulation is supplied as a suspension. In some embodiments, a suspension is a solution. In some embodiments, the suspension is refrigerated. In some embodiments, the suspension is frozen. In some embodiments, methods provided herein further comprise the step of warming the refrigerated suspension to room temperature and/or agitating the suspension to ensure that the active ingredient(s) are dissolved and/or evenly distributed in solution prior to administering to the one eye and/or the contralateral eye of the individual (e.g., via IVT injection). In some embodiments, methods provided herein further comprise the step of thawing the frozen suspension and warming to room temperature and/or agitating the suspension to ensure that the active ingredient(s) are dissolved and/or evenly distributed in solution prior to administering to the one eye and/or the contralateral eye of the individual (e.g., via IVT injection). In some embodiments, the suspension is diluted prior to administration to the subject (e.g., via IVT injection). In some embodiments, the suspension is supplied as a pre-filled syringe.

In some embodiments, the pharmaceutical formulation is provided as a frozen suspension. In some embodiments, the suspension comprises a pharmaceutically acceptable excipient, e.g., surfactant, glycerol, non-ionic surfactant, buffer, glycol, salt, and any combination thereof.

In some embodiments, the suspension is a solution. In some embodiments, the suspension comprises micelles.

In some embodiments, for storage stability and convenience of handling, a pharmaceutical formulation, comprising rAAV (e.g., AAV2.7m8) and a nucleic acid sequence that encodes the anti-VEGF agent (e.g., aflibercept or a functional fragment or variant thereof), is formulated as a lyophilized, freeze dried, or vacuum dried powder that is reconstituted with saline, buffer, or water prior to administration to the one eye and/or the contralateral eye of the individual. Alternately, the pharmaceutical formulation is formulated as an aqueous solution, such as a suspension or a homogeneous solution. A pharmaceutical formulation can contain rAAV particles comprising a nucleic acid sequence that encodes aflibercept. Various excipients, such as phosphate, PBS, or Tris buffer, glycol, glycerol, saline, surfactant (e.g., pluronic or polysorbate), or any combination thereof, can be used to stabilize a pharmaceutical formulation. Additionally, cryoprotectants, such as alcohols can be used as a stabilizer under freezing or drying conditions. In some embodiments, the gene therapy is provided as a suspension, a refrigerated suspension, or a frozen suspension.

In some embodiments, a suspension of the pharmaceutical formulation as disclosed herein has a volume of any of about 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, or 1000 μL. In some embodiments, a suspension of the pharmaceutical formulation as disclosed herein has a volume of about 250 μL. In some embodiments, the suspension of the pharmaceutical formulation as disclosed herein has a volume of between 0.1 to 0.5 mL, between 0.1 to 0.2 mL, between 0.3 to 0.5 mL, between 0.5-1.0 mL, between 0.5-0.7 mL, between 0.6 to 0.8 mL, between 0.8 to 1 mL, between 0.9 to 1.1 mL, between 1.0 to 1.2 mL, or between 1.0 to 1.5 mL. In other embodiments, the volume is no more than 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL, or 1.5 mL. In some embodiments, the suspension of the pharmaceutical formulation as disclosed herein has a volume of about 0.25 mL.

In some embodiments, a suspension of the pharmaceutical formulation as disclosed herein is provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal). In some embodiments, a suspension of the pharmaceutical formulation as disclosed herein is provided as a sterile-filtered, frozen suspension in a sterile, ready-to-use vial (e.g., a 0.5 mL vial; e.g., a Crystal Zenith® vial) with, a ready-to-use stopper (e.g., a stopper made of chlorobutyl), and sealed (e.g., with a sterile aluminum tear-off seal), wherein the vial contains a volume of between 0.1 to 0.5 mL, between 0.1 to 0.2 mL, between 0.2 to 0.3 mL, between 0.3 to 0.4 mL, or between 0.4 mL to 0.5 mL of the suspension of the pharmaceutical formulation. In some embodiments, a suspension of the pharmaceutical formulation as disclosed herein is provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal), wherein the vial contains a volume of about 0.25 mL of the suspension of the pharmaceutical formulation.

In some embodiments, pharmaceutical formulations disclosed herein are designed, engineered, or adapted for administration to a primate (e.g., non-human primate and human subjects) via intravitreal or subretinal injection. In some embodiments, a pharmaceutical formulation comprising rAAV particles comprising a nucleic acid sequence that encodes the anti-VEGF agent (e.g., aflibercept) is formulated for intravitreal injection into an eye of an individual. In some embodiments, the pharmaceutical composition is formulated to or reconstituted to a concentration that allows intravitreal injection of a volume not more than about or not more than any of 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL. In some embodiments, a unit dose of the pharmaceutical formulation comprises a volume not more than about or not more than any of 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL. In some embodiments, methods disclosed herein comprise intravitreal injection of a volume of any of about 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL of a solution or suspension of a pharmaceutical formulation comprising a rAAV (e.g., AAV2.7m8) and a nucleic acid sequence that encodes the anti-VEGF agent (e.g., aflibercept). In some embodiments, methods disclosed herein comprise intravitreal injection of a volume of about 30 μL or about 100 μL of a solution or suspension of a pharmaceutical formulation comprising a rAAV (e.g., AAV2.7m8) and a nucleic acid sequence that encodes the anti-VEGF agent (e.g., aflibercept). In some embodiments, methods disclosed herein comprise intravitreal injection of a volume of about 30 μL of a solution or suspension of a pharmaceutical formulation comprising a rAAV (e.g., AAV2.7m8) and a nucleic acid sequence that encodes the anti-VEGF agent (e.g., aflibercept). In some embodiments, methods disclosed herein comprise intravitreal injection of a volume of about 100 μL of a solution or suspension of a pharmaceutical formulation comprising a rAAV (e.g., AAV2.7m8) and a nucleic acid sequence that encodes the anti-VEGF agent (e.g., aflibercept).

In some embodiments, an AAV2.7m8 particle comprising a nucleic acid sequence of the anti-VEGF agent (e.g., aflibercept) transgene described herein is a component of a gene therapy pharmaceutical formulation. In some embodiments, a rAAV particle of any serotype comprising the 7m8 variant capsid protein as described herein is used to make a frozen suspension or a freeze-dried or lyophilized formulation composition. In some embodiments, the gene therapy is formulated as a refrigerated or frozen suspension. In some embodiments, the rAAV particle is rAAV2. In some embodiments, the lyophilized or suspension of the pharmaceutical formulation comprises rAAV2 comprising the 7m8 variant capsid protein and a DNA sequence that encodes the anti-VEGF agent (e.g., aflibercept). In some embodiments, the suspension is refrigerated or frozen.

In some embodiments, the administration of the unit dose of rAAV particles to the one eye and/or to the contralateral eye of the individual is by intravitreal (IVT) injection. For IVT injection, the rAAV particles can be delivered in the form of a suspension of a pharmaceutical formulation (e.g., as described herein). Initially, topical anesthetic is applied to the surface of the eye followed by an ophthalmic antiseptic solution. The eye is held open, with or without instrumentation, and the rAAV particles are injected through the sclera with a short, narrow needle, e.g., a 30-gauge needle, into the vitreous cavity of the one eye and/or the contralateral eye of the individual under direct observation. Typically, a volume of between about 25 μL to about 250 μL (e.g., any of about 25 μL, about 30 μL, about 40 μL about 50 μL, about 60 μL, about 70 μL, about 80 μL, about 90 μL, about 100 μL, about 10 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, or about 250 μL) of an rAAV particle suspension may be delivered to the eye by IVT injection. In some embodiments, the unit dose of rAAV particles comprises a volume of about 100 μL. In some embodiments, the unit dose of rAAV particles comprises a volume of about L. In some embodiments, the IVT injection is performed in combination with removal of vitreous fluid. In some embodiments, a vitrectomy may be performed, and the entire volume of vitreous gel is replaced by an infusion of the rAAV particle suspension (e.g., about 4 mL of the rAAV particle suspension). A vitrectomy is performed using a cannula of appropriate bore size (e.g., 20 gauge to 27 gauge), wherein the volume of vitreous gel that is removed is replaced by infusion of fluid, e.g., saline, an isotonic solution, a rAAV particle suspension, from the infusion cannula. IVT administration is generally well tolerated. At the conclusion of the procedure, there is sometimes mild redness at the injection site. There is occasional tenderness, but most patients do not report any pain. No eye patch or eye shield is necessary after this procedure, and activities are not restricted. Sometimes, an antibiotic eye drop is prescribed for several days to help prevent infection.

In some embodiments, the pharmaceutical formulation is a unit dose (e.g., a therapeutically effective dose) to be administered to the one eye and/or the contralateral eye of an individual (e.g., a human or non-human primate) via IVT injection for the treatment of an ocular disease or disorder characterized by abnormal (e.g., excessive) angiogenesis or neovascularization. In some embodiments, the pharmaceutical formulation comprises a unit dose (e.g., a therapeutically effective dose) as described in further detail elsewhere herein. In some embodiments, the volume of the unit dose (e.g., a therapeutically effective dose) of a viral vector (e.g., an rAAV vector disclosed herein) administered to the subject is no more than any one of about 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL, including any range in between these values. Minimizing the volume of the unit dose to be administered to the subject may obviate or mitigate changes in ocular pressure and other adverse effects associated with IVT injection (e.g., elevated intraocular pressure, inflammation, irritation, or pain).

Pharmaceutical formulations suitable for ocular use include sterile aqueous solutions or dispersions and sterile powders for the extemporaneous preparation of sterile injectable solutions, suspension, or dispersion. For intravitreal administration, suitable carriers include physiological saline, bacteriostatic water, phosphate buffered saline (PBS), and/or an isotonic agent, e.g., glycerol. In certain embodiments, the pharmaceutical formulation is sterile and fluid to the extent that easy syringability or injectability exists. In certain embodiments, the pharmaceutical formulation is stable under the conditions of manufacture and storage and is preserved against the contaminating action of microorganisms such as bacteria and fungi. In some embodiments, the pharmaceutical composition can include an isotonic agent, such as a salt or glycerol. In some embodiments, a surfactant or a stabilizer is added to the pharmaceutical composition to prevent aggregation.

In some embodiments, the pharmaceutical formulation contains an excipient or a carrier. A carrier is a solvent or dispersion medium containing, for example, water, saline, ethanol, a polyol (for example, glycerol, propylene glycol, and liquid polyethylene glycol, and the like), and any combination thereof. The proper fluidity can be maintained, for example, by the use of a coating such as lecithin, by the maintenance of the required particle size in the case of dispersion and by the use of surfactants such as polysorbates (e.g., Tween™, polysorbate 20, polysorbate 80), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™) N,N-dimethyldodecylamine-N-oxide, hexadecyltrimethylammonium bromide (HTAB), polyoxyl 10 lauryl ether, Brij 721™, bile salts (sodium deoxycholate, sodium cholate), pluronic acids (F-68, F-127), polyoxyl castor oil (Cremophor™) nonylphenol ethoxylate (Tergitol™), cyclodextrins, and ethylbenzethonium chloride (Hyamine™). Prevention of the action of microorganisms can be achieved by various antibacterial and antifungal agents, for example, parabens, chlorobutanol, phenol, ascorbic acid, cresol, thimerosal, and the like. In many embodiments, isotonic agents are included in the pharmaceutical formulation, for example, sugars, polyalcohols such as mannitol, sorbitol, and/or sodium chloride. Prolonged absorption of the internal compositions can be brought about by including in the composition an agent that delays absorption, for example, aluminum monostearate and gelatin. In some embodiments, the pharmaceutical carrier includes sodium phosphate, sodium chloride, polysorbate, and sucrose. In some embodiments, a pharmaceutical formulation comprises a surfactant, e.g., non-ionic surfactant such as polysorbate, poloxamer, or pluronic. In some embodiments, the addition of a non-ionic surfactant reduces aggregation in the pharmaceutical composition.

Also provided herein are kits comprising at least one pharmaceutical formulation described herein. In some embodiments, the kit comprises a frozen suspension of a pharmaceutical formulation (e.g., one unit dose in a vial). In some embodiments, the kit comprises a lyophilized or freeze-dried pharmaceutical formulation (e.g., one unit dose in a vial) disclosed herein and a solution for dissolving, diluting, and/or reconstituting the lyophilized pharmaceutical composition. In some embodiments, the solution for reconstituting or dilution is supplied as a pre-filled syringe. In some embodiments, a kit comprises a freeze-dried or lyophilized pharmaceutical composition comprising rAAV (e.g., AAV2.7m8) and a solution for reconstituting the pharmaceutical composition to a desired concentration or volume. In some embodiments, the kit includes a buffer that helps to prevent aggregation upon reconstituting the pharmaceutical composition disclosed herein. In some embodiments, the pharmaceutical composition is provided in a pre-filled syringe. In some embodiments, a kit comprises a dual-chamber syringe or container wherein one of the chambers contains a buffer for dissolving or diluting the pharmaceutical composition. In some embodiments, the kit comprises a syringe for injection. In some embodiments, the reconstituted solution is filtered before administration. In some embodiments, the kit comprises a filter or a filter syringe for filtering the reconstituted pharmaceutical composition before administration to a patient. In some embodiments, the kit comprises a suspension of the pharmaceutical formulation comprising the rAAV particles as disclosed herein provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal). In some embodiments, the kit comprises a suspension of the pharmaceutical formulation comprising the rAAV particles as disclosed herein provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal), wherein the vial contains a volume of between 0.1 to 0.5 mL, between 0.1 to 0.2 mL, between 0.2 to 0.3 mL, between 0.3 to 0.4 mL, or between 0.4 mL to 0.5 mL of the suspension of the pharmaceutical formulation. In some embodiments, the kit comprises a suspension of the pharmaceutical formulation comprising the rAAV particles as disclosed herein provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal), wherein the vial contains a volume of about 0.25 mL of the suspension of the pharmaceutical formulation. In some embodiments, the kit further comprises instructions for use, e.g., instructions for treating an ocular disease with the rAAV particles disclosed herein.

### Ocular Diseases

In one aspect, the present disclosure provides methods for treating an ocular disease in an individual. In another aspect, the present disclosure provides methods for reducing intraocular pressure (IOP) in the eye of an individual with an ocular disease.

In some embodiments, the ocular disease glaucoma. As used herein, “glaucoma” refers to eye conditions that damage the optic nerve and cause vision loss. Typically, this damage is caused by an abnormally high pressure in the eye. Glaucoma is one of the leading causes of blindness for people over the age of 60. It can occur at any age but is more common in older adults. In some embodiments, the glaucoma is a primary glaucoma, also known as chronic glaucoma. Primary glaucoma is caused by excess pressure in the eye, known as intraocular pressure (IOP). This increase in pressure is usually due to improper drainage of fluid within the eye. In some embodiments, the glaucoma is a secondary glaucoma. Secondary glaucoma refers to any form of glaucoma in which there is an identifiable cause of increased eye pressure, resulting in optic nerve damage and vision loss. In some embodiments, the glaucoma is neovascular glaucoma, which is typically caused by the abnormal formation of new blood vessels on the iris and over the eye's drainage channels. The new blood vessels block the eye's fluid from exiting through the trabecular meshwork, causing an increase in eye pressure. Neovascular glaucoma is always associated with other abnormalities, most often diabetes. In some embodiments, the glaucoma is open-angle (wide angle, chronic simple) glaucoma, in which the drainage angle for fluid within the eye remains open, with less common types including closed-angle (narrow angle, acute congestive) glaucoma and normal-tension glaucoma. Open-angle glaucoma develops slowly over time and there is no pain. Peripheral vision may begin to decrease, followed by central vision, resulting in blindness if not treated. In some embodiments, the glaucoma is closed angle glaucoma. Closed-angle glaucoma may present gradually or suddenly. The sudden presentation of closed-angle glaucoma may involve severe eye pain, blurred vision, mid-dilated pupil, redness of the eye, and nausea. Vision loss from glaucoma, once it has occurred, is permanent. If treated early, it is possible to slow or stop the progression of disease with medication, laser treatment, or surgery. The goal of treatment is to decrease eye pressure

In some embodiments, the individual received at least one prior treatment (e.g., at least one, at least two, at least three, at least four, at least 5 or more treatments) with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in about the last 12 weeks (e.g., about 3 or about 4 months) prior to administration of the unit dose of rAAV particles. In some embodiments, the individual received 2 or 3 prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye during about the last 12 weeks (e.g., about 3 or about 4 months) prior to administration of the unit dose of rAAV particles to the one eye and/or the contralateral eye. In some embodiments, the individual received at least about 1, at least about 5, at least about 10, at least about 20, at least about 30, at least about 40, at least about 50, at least about 60, at least about 70, at least about 80, at least about 90, at least about 100, at least about 10, at least about 120, or more prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or the contralateral eye. In some embodiments, the individual had a calculated anti-VEGF agent (e.g., bevacizumab, brolucizumab, ranibizumab, and/or aflibercept) injection interval in the one eye and/or the contralateral eye of about 2 weeks, about 3 weeks, 4 weeks, about 5 weeks, about 6 weeks, about 7 weeks, about 8 weeks, about 9 weeks, about 10 weeks, about 11 weeks, about 12 weeks, or more. In some embodiments, the individual had a calculated anti-VEGF (e.g., bevacizumab, brolucizumab, ranibizumab, and/or aflibercept) injection interval in the one eye and/or the contralateral eye of about 5-7 weeks, about 4-10 weeks, about 4-7 weeks, or about 4-6 weeks. In some embodiments, the individual received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye any of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days prior to administration of the unit dose of rAAV particles to the one eye and/or the contralateral eye. In some embodiments, the individual received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or the contralateral eye about 7 days, about 10 days, or about 14 days prior to administration of the unit dose of rAAV particles to the one eye and/or the contralateral eye. In some embodiments, the prior treatment comprises an intraocular, subretinal or intravitreal injection with an anti-VEGF agent. In some embodiments, the anti-VEGF agent is bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept. In some embodiments, the anti-VEGF agent is aflibercept. In some embodiments, the individual received between 1 and 20 (e.g., any of 1, 2, 3, 4, 5, 6, 7, 8, 9, 10, 11, 12, 13, 14, 15, 16, 17, 18, 19, or 20) prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye during the last about 12 months prior to administration of the unit dose of rAAV particles to the one eye and/or the contralateral eye. In some embodiments, the individual received about 9 or about 10 prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye during the last about 12 months prior to administration of the unit dose of rAAV particles to the one eye and/or the contralateral eye.

In some embodiments, the individual has not received a prior treatment for an ocular disease. In some embodiments, the individual has not received a prior treatment in the one eye and/or the contralateral eye for an ocular disease. In some embodiments, the individual has not received a prior anti-VEGF treatment. In some embodiments, the individual has not received a prior anti-VEGF treatment in the one eye and/or the contralateral eye. In some embodiments, the individual has not received a prior aflibercept treatment. In some embodiments, the individual has not received a prior aflibercept treatment in the one eye and/or the contralateral eye.

In some embodiments, methods described herein are used to prevent or treat an ocular disease or disorder in a subject who has received prior treatment with bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept. In some embodiments, methods described herein are used to prevent or treat an ocular disease or disorder that is responsive to treatment with bevacizumab, brolucizumab, ranibizumab, and/or aflibercept.

In some embodiments, the individual was diagnosed with the ocular disease at least 1 day, at least 1 week, at least 1 month, at least 2 months, at least 4 months, at least 6 months, at least 12 months, at least 18 months, at least 24 months, at least 30 months, at least 36 months, at least 42 months, at least 48 months, at least 54 months, at least 60 months, at least 66 months, at least 72 months, at least 78 months, at least 84 months, at least 90 months, 96 months, at least 102 months, at least 108 months, at least 114 months, at least 120 months, at least 126 months, at least 132 months, or more, prior to administration of the unit dose of rAAV particles to the one eye and/or the contralateral eye.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: A Phase 2, Multi-Center, Randomized, Double-Masked, Active Controlled Study of AAV2.7m8-aflibercept in Subjects with Diabetic Macular Edema

This Example describes a Phase 2, multi-center, randomized, double-masked, active controlled study designed to evaluate the durability of a single intravitreal (IVT) injection of AAV2.7m8-aflibercept in subjects with diabetic macular edema.

### I. Study Objectives and Endpoints

### A. Primary Objective

The primary objective of this study was to assess the durability of a single IVT injection of AAV2.7m8-aflibercept.

### B. Secondary Objectives

Secondary objectives of this study included:
Assessment of the safety and tolerability of AAV2.7m8-aflibercept.
Evaluation of the effect of AAV2.7m8-aflibercept on macular edema.
Evaluation of the effect of AAV2.7m8-aflibercept on Best Corrected Visual Acuity (BCVA).
Evaluation of the effect of AAV2.7m8-aflibercept on Diabetic Retinopathy Severity Scale (DRSS) score.
Assessment of the need for rescue aflibercept (2 mg IVT).
Assessment of the effect of a preceding dose of aflibercept (2 mg IVT) prior to AAV2.7m8-aflibercept administration.
Evaluation of the effect of AAV2.7m8-aflibercept on development of vision threatening complications (anterior segment neovascularization, vitreous hemorrhage, or tractional retinal detachment).

### C. Primary Endpoints

The primary endpoint of this study was the time to worsening of diabetic macular edema (DME) disease activity in the study eye, as defined by the occurrence of either of:
An increase in central subfield thickness (CST) >50 m as assessed by SD-OCT compared to the lower of the two CST measurements recorded at Day 1 or Week 4.
A loss of >5 letters in BCVA due to worsening DME disease activity compared to the higher of the two BCVA measurements recorded at Day 1 or Week 4.

### D. Secondary Endpoints

The secondary endpoints for this study were based on outcome measures for the study eye (unless otherwise specified) and included:
Incidence and severity of ocular and non-ocular adverse events (AEs).
Change from Baseline in CST and macular volume over time through Week 48.
Change from Baseline in BCVA over time through Week 48.
Frequency of rescue aflibercept (2 mg IVT) in the study eye over time during the study.
Incidence of 2-step and 3-step improvement in DRSS score over time through Week 48.
Incidence of 2-step and 3-step worsening in DRSS score over time through Week 48.
Occurrence of vision threatening complication (anterior segment neovascularization, vitreous hemorrhage, or any other high-risk proliferative diabetic retinopathy (DR), or tractional retinal detachment) over time through Week 48.
Incidence of CST <300 m over time through Week 48.
Incidence of clinically significant findings via physical examinations, ocular examinations, imaging, and laboratory evaluation over time through Week 48.

### II. Study Population

### A. Inclusion Criteria

Subjects with newly diagnosed DME (i.e., DME diagnosis within 6 months of screening) that received up to 2 prior injections of anti-VEGF therapy in the study eye were included in this study if they met the following inclusion criteria:
Age >18 years of age.
Type 1 or 2 diabetes mellitus.
Vision impairment due to center involving diabetic macular edema.
Vision at Screening:
   Study Eye: BCVA 78 to 50 ETDRS letters, inclusive (approximate Snellen equivalent 20/32 to 20/100).
   Non-study eye: BCVA 35 ETDRS letters or more (approximate Snellen equivalent of 20/200 or better).
CST of study eye at Screening visit of ≥325 m using Heidelberg Spectralis® with center-involving IRF (center 1 mm).
A decrease in vision in the study eye determined to be primarily due to DME.
Initial DME diagnosis within 6 months from screening.
Up to 2 prior injections (0, 1, or 2) of anti-VEGF in the study eye.
   If a prior anti-VEGF has been administered to the study eye, there must have been a meaningful CST response (e.g., >10% reduction) and no adverse reaction to anti-VEGF (e.g., inflammation).
A minimum 60-day interval between the last anti-VEGF injection in the study eye and randomization on Day 1.

### B. Exclusion Criteria

Subjects meeting any of the following criteria were excluded from this study:
Documented anti-AAV2.7m8 neutralizing antibody titer >1:125 within 6 months prior to randomization.
Prior ocular gene therapy.
History of allergy to aflibercept, corticosteroid, or fluorescein dye or sodium fluorescein used in angiography (mild allergy amenable to treatment was allowable).
History or evidence of any of the following cardiovascular disease within 6 months of dosing:
   Severe cardiac disease (e.g., New York Heart Association [NYHA] Functional Class III or IV) or clinical evidence of unstable angina.
   Acute coronary syndrome, myocardial infarction or coronary artery revascularization, cerebrovascular accident (CVA), transient ischemic attack (TIA).
   Ventricular tachyarrhythmias requiring ongoing treatment, or uncontrolled arrhythmia.
   Uncontrolled hypertension defined as systolic blood pressure (SBP) >160 mmHg or a diastolic blood pressure (DBP) >100 mmHg, despite using BP-lowering medication within the screening period. If BP-lowering medications were required, subject should have been on a stable dose of the same medication continuously for 30 days prior to randomization.
Any history of ongoing bleeding disorders. The use of aspirin or other anticoagulants (e.g., Factor Xa inhibitors) was not an exclusion criterion.
Uncontrolled diabetes defined as HbA1C >10%; or history of diabetic ketoacidosis within 3 months prior to randomization; or subjects who, within the last 3 months, initiated intensive insulin treatment (a pump or multiple daily injection) or planned to do so in the next 3 months.
History of systemic autoimmune disease requiring treatment with systemic steroids or immunosuppressive treatments (e.g. methotrexate, adalimumab).
Systemic drugs known to cause macular edema (e.g., fingolomod, tamoxifen, chloroquine/hydroxychloroquine) or any prior systemic anti-VEGF therapy.
Known to be positive for HIV or active viral hepatitis (unless documented cure after treatment for Hepatitis C); known history of syphilis
Known severe renal impairment, as indicated by estimated CrCl<30 mL/min (by Cockcroft-Gault calculation); need or anticipated need for hemodialysis during the study period.
Any febrile illness within 1 week prior to randomization.

In addition, subjects meeting any of the following ocular exclusion criteria in the study eye were excluded from this study:
High-risk proliferative diabetic retinopathy (PDR) at time of screening, defined as: any vitreous or preretinal hemorrhage, neovascularization elsewhere >½-disc area within an area equivalent to standard ETDRS 7-field on clinical examination, or neovascularization of disc >⅓-disc area on clinical examination.
Any prior focal or grid laser photocoagulation or any prior pan retinal photocoagulation (PRP) in the study eye.
Any anti-VEGF therapy in the preceding 60 days prior to randomization (up to 2 prior anti-VEGF injections were allowed but they cannot have occurred in the prior 60 days).
History of anterior segment neovascularization (e.g., neovascularization of the iris [NVI] or neovascular glaucoma [NVG]), significant vitreous hemorrhage, fibrovascular proliferation or tractional retinal detachment.
Examination evidence of structural abnormalities at the fovea (e.g., dense hard exudates, pigment abnormalities, foveal atrophy, vitreomacular traction or epiretinal membrane), either on clinical examination or OCT, thought to be contributing to macular edema or visual impairment.
History of retinal disease in the study eye other than diabetic retinopathy including age-related macular degeneration (in either eye), retinal vein occlusion, retinal arterial occlusion, pathologic myopia, etc.
Any current or history of ocular disease other than DME that could reduce the potential for visual improvement, confound assessment of the macula or require medical or surgical intervention during the study (e.g. significant cataract, macular traction) or any evidence of posterior subcapsular cataract.
History of cataract extraction or Yttrium Aluminum Garnet (YAG) capsulotomy within 3 months before Day 1.
History of retinal detachment (with or without repair) in the study eye.
History of trabeculectomy or glaucoma shunt or minimally invasive glaucoma surgery (MIGS).
History of vitrectomy or other filtration surgery.
Aphakia or presence of an anterior chamber intraocular lens.
Uncontrolled ocular hypertension or glaucoma in the study eye at time of randomization (defined as IOP >22 mmHg despite treatment with anti-glaucoma medication) or current use of >2 IOP lowering medications.
Any history of intraocular or periocular steroid treatment for any ocular condition (e.g., IVT Triesence, Iluvien or Ozurdex).
Refractive surgery within the 90 day period prior to Screening.
Previous penetrating keratoplasty, endothelial keratoplasty, or ocular radiation.
Any prior vitreoretinal surgery.

In addition, subjects meeting any of the following ocular exclusion criteria in the study eye or in the non-study eye (i.e., the “fellow eye”) were excluded from this study:
Any history of uveitis or intraocular inflammation (grade trace or above) except mild anticipated post-operative inflammation that resolved.
History of IOP elevation related to topical steroid administration.
Known history of ocular Herpes Simplex Virus (HSV), Varicella-zoster virus (VZV), or Cytomegalovirus (CMV) including viral uveitis, retinitis or keratitis.
Evidence of external ocular infection, including conjunctivitis, chalazion or significant blepharitis.
History of ocular toxoplasmosis.

### III. Study Design

### A. Study Treatment

This was a multi-center, randomized, double-masked, controlled, parallel-group study designed to evaluate the efficacy, safety and tolerability of a single 0.10 mL IVT injection of AAV2.7m8-aflibercept. Two doses of AAV2.7m8-aflibercept were investigated.

Subjects with initial diagnosis of DME within 6 months of screening and that have received up to 2 prior injections of anti-VEGF therapy were eligible for enrollment.

Approximately 33 eligible subjects were randomized to receive one of two doses of AAV2.7m8-aflibercept (6×10¹¹ vg/eye or 2×10¹¹ vg/eye), or to a control arm to receive a sham ocular injection with a preceding aflibercept injection. Subjects who were assigned to receive AAV2.7m8-aflibercept were further randomized to receive a preceding aflibercept or sham ocular injection. The study arms are summarized below:
Arm 1 (n=6): Subjects receive AAV2.7m8-aflibercept at a dose of 6×10¹¹ vg/eye with a preceding aflibercept dose.
Arm 2 (n=6): Subjects receive AAV2.7m8-aflibercept at a dose of 6×10¹¹ vg/eye without a preceding aflibercept dose.
Arm 3 (n=6): Subjects receive AAV2.7m8-aflibercept at a dose of 2×10¹¹ vg/eye with a preceding aflibercept dose.
Arm 4 (n=6): Subjects receive AAV2.7m8-aflibercept at a dose of 2×10¹¹ vg/eye without a preceding aflibercept dose.
Arm 5 (n=9): Subjects receive aflibercept only (active control).

To maintain masking of the treatment assignment, subjects assigned to the arms with no preceding aflibercept on Day 1 or to the arm with no AAV2.7m8-aflibercept on Day 8 received a sham ocular injection on the corresponding visit. Only one eye per subject was selected as the study eye. If both eyes were eligible, the eye with the worse BCVA was selected as the study eye.

Both AAV2.7m8-aflibercept and aflibercept were administered via IVT injection. IVT injections were not performed if active inflammation was present. Aseptic technique with povidone-iodine was used with topical or subconjunctival anesthesia. The sham ocular injection procedure was done under the same conditions but with an empty syringe without a needle (using the blunt end) pressed against the eye to mimic an injection.

A summary of the arms in this study is provided in Table 17.

TABLE 17
Study Arms.

| | | | Day 8 |
|---|---|---|---|
| | | Day 1 | AAV2.7m8- |
| | | IVT Aflibercept | aflibercept Dose |
| Arm | N | or Sham | (vg/eye) |
| 1 | 6 | Aflibercept | 6 × 10¹¹ |
| 2 | 6 | Sham | 6 × 10¹¹ |
| 3 | 6 | Aflibercept | 2 × 10¹¹ |
| 4 | 6 | Sham | 2 × 10¹¹ |
| 5 | 9 | Aflibercept | Sham |

After the assigned IVT injections on Day 1 and Day 8, all subjects were followed up on Week 2, Week 4, and then every 4 weeks up to Week 48 after Day 1 (e.g., Week 8, Week 12, Week 16, etc.). To maintain masking, both subjects and personnel conducting assessments were masked to the treatment assignments throughout the study.

All subjects were followed for 48 weeks after randomization.

### B. Prophylactic Topical Steroid Regimen

All subjects were administered a prophylactic 7-week topical corticosteroid regimen of difluprednate (0.05%; e.g., Durezol) starting on Day 1. Subjects were instructed to self-administer difluprednate four times per day (QTD) for 4 weeks (i.e., from Day 1 to Day 28), followed by three times per day (TID) for 1 week (i.e., for 7 days), followed by two times per day (BID) for 1 week (i.e., 7 days), and finally once per day (QD) for 1 week (i.e., 7 days). Tapering was not commenced in the presence of active inflammation. This regimen was prolonged should signs of inflammation have occurred. A summary of the difluprednate regimen is provided in Table 2.

TABLE 2
Difluprednate Regimen.

| | Total | Difluprednate |
|---|---|---|
| | Number | Administrations |
| Study Days | of Days | per Day |
| Days 1 to 28 | 28 | 4 times |
| Days 29 to 42 | 14 | 3 times |
| Days 43 to 56 | 14 | 2 times |
| Days 57 to 70 | 14 | 1 time |
| Day 71 | 0 | STOP - if no signs |
| | | of inflammation |

### C. Rescue Treatment

Starting at Week 8, subjects received rescue aflibercept (2 mg IVT) if they met any of the following:
Increase in CST >50 m as assessed by SD-OCT compared to the lower of the two CST measurements recorded at Day 1 or Week 4.
Loss of >5 letters in BCVA due to worsening DME disease activity compared to the higher of the two BCVA measurements recorded at Day 1 or Week 4.

Aflibercept was not injected in eyes with active inflammation. A minimum of 21 days was required between rescue aflibercept injections.

### D. Medications and Treatments

The following medications were prohibited during the study:
Any systemic anti-VEGF agent, including bevacizumab.
Systemic drugs known to cause macular edema (e.g., fingolomod, tamoxifen, chloroquine/hydroxychloroquine).
Any anti-VEGF agent in the study eye other than the study drug or aflibercept IVT 2 mg.
IVT steroids in the study eye (e.g., Ozurdex or Illuvien Triesence).
Systemic immunosuppressive drugs (e.g., intravenous steroids, methotrexate, azathioprine, ciclosporin, adalimumab, infliximab, etanercept). Inhaled or topical steroids and NSAIDs were allowed.

Subjects who developed high-risk PDR in the study eye received panretinal photocoagulation (PRP) after receiving rescue aflibercept.

Subjects with visually significant cataract were not enrolled in the study, but if a cataract developed during the study, cataract surgery in the study eye was permitted if clinically indicated and was scheduled >90 days after AAV2.7m8-aflibercept administration and/or >7 days after the last injection of aflibercept.

Subjects who developed DME in the fellow (non-study) eye were permitted to receive standard of care therapy.

### IV. Study Assessments

### A. General Physical Examination and Vital Signs

A general physical examination (PE) was conducted at the screening and End of Study (EOS) or Early Termination visit. The PE consisted of body system examination for general appearance, neurologic, HEENT (head, eyes, ears, nose, and throat), neck, cardiovascular, respiratory, abdomen, extremities, skin, weight, and height. At the EOS or Early Termination visit, the physical examination assessed if any changes in the subject's physical condition had occurred since the Screening examination. A targeted physical examination was conducted as needed for the evaluation of AEs.

Vital signs consisted of blood pressure, pulse rate, body temperature, and respiratory rate. A 12-lead Electrocardiogram (ECG) was taken for each subject at Screening and EOS or Early Termination Visit.

### B. Laboratory Tests, Vector Expression and Immune Response

The following Clinical Laboratory Tests were conducted for the study: chemistry, complete blood count, HbAlC, urinalysis, and HLA-B27 genotyping.

Subjects' samples (both blood and/or aqueous humor) were collected to measure the following:
Total antibodies to AAV2.7m8: serum for total anti-AAV2.7m8 antibodies were measured in an ELISA assay.
Neutralizing antibodies to AAV2.7m8: serum for neutralizing anti-AAV2.7m8 antibodies were measured in a cell-based assay.
Anti-aflibercept antibodies: serum for the humoral immune response against aflibercept was measured in an ELISA assay.
Aflibercept protein expression: Serum and aqueous humor samples were collected for the presence of aflibercept protein and measured in a MesoScale Discovery assay.
Cell-mediated immune response: cellular immunity against AAV2.7m8 capsid and aflibercept protein were measured in an ELISPOT assay.

### C. Full Ophthalmic Examination

Study assessments included an ophthalmologic exam, Intraocular Pressure (IOP), and indirect ophthalmoscopy.

The ophthalmic examination consisted of an external examination of the eye and adnexa, routine screening for eyelid/pupil responsiveness (including but not limited to blepharoptosis, abnormal pupil shape, unequal pupils, abnormal reaction to light, and afferent pupillary defect), and slit-lamp examination (eyelids, conjunctiva, cornea, lens, iris, anterior chamber). The slit-lamp examination examined the anterior ocular structures and was used for grading any findings. If any finding was noted during the slit-lamp examination, at any visit, the severity was graded and the finding was described as clinically significant or not clinically significant.

IOP measurements were performed using a Goldmann applanation tonometer or Tono-pen™. The same method of IOP measurement was used throughout the study for each individual subject. IOP measurements were performed prior to any IVT injection and prior to dilating eyes, using the same method throughout the study. Day 1 and Day 8 visits required pre-injection and post-injection (30 minutes after injection) IOP measurements.

The dilated indirect ophthalmoscopy examination included an evaluation of posterior segment abnormalities of the vitreous, optic nerve, peripheral retina, and retinal vasculature. If any finding was noted during the ophthalmoscopy, at any visit, the severity was graded and the finding was described as clinically significant or not clinically significant. Day 1 and Day 8 visits required pre-injection and post-injection indirect ophthalmoscopy assessments.

### D. Refraction and Visual Acuity

Refraction and BCVA were measured. Visual acuity measurements were measured at a starting distance of 4 meters, prior to dilating eyes.

### E. Imaging

Spectral Domain Optical Coherence Tomography (SD-OCT) is an interferometric technique that provides depth-resolved tissue structure information encoded in the magnitude and delay of the back-scattered light by spectral analysis of the interference fringe pattern. If a subject received anti-VEGF injections at visits prior to study randomization, OCTs from those visits were collected and delivered to the central reading center.

Optical Coherence Tomography Angiography (OCT-A) is an imaging technology that provides volumetric, three-dimensional maps of the retina and choroid as well as information on blood flow. There are two types of OCT-A, swept-source and spectral-domain. Swept-source imaging was used where available. If a swept-source instrument was not available and a spectral-domain instrument was available, then the spectral domain instrument was used.

Standardized procedures for the collection of ultra-wide field fundus digital photographic images of the retina, optic disc, and macula were followed. In addition, photographs of the iris were taken prior to dilation.

A standardized procedure for examining the retinal circulation and vessel permeability using a dye-tracing method was followed. This involved injection of sodium fluorescein into the systemic circulation, after which an angiogram was obtained by digitally photographing the fluorescence emitted after illumination of the retina with blue light at a wavelength of 490 nm.

### F. Laboratory, Biomarker and Other Biological Specimens

Aqueous humor samples were collected and analyzed for levels of aflibercept, VEGF-A, neutralizing antibodies (NAbs), and additional biomarkers. Vitreous humor samples were obtained and analyzed for aflibercept concentrations and other biomarkers.

### G. Safety

After study treatment administration, all clinically significant adverse events (AEs) were reported. Each subject was followed until a) the end of the AE reporting period at 30 days after the last study visit or b) for any ongoing study treatment related AEs and/or serious AEs (SAEs) until resolved or stable. Any clinically significant safety assessment that was associated with DME was not reported as an AE or SAE, unless judged to be more severe than expected for the subject's condition. Progression of the disease under study was captured as an efficacy outcome.

Adverse events of special interest for this study included:
Sight-threatening adverse events: an adverse event was considered to be sight-threatening if it met one or more of the following criteria:
   Caused a decrease of >30 letters in BCVA compared with the prior visit.
   Required surgical or medical intervention (i.e., conventional surgery, vitrectomy) to prevent permanent loss of sight.
   Caused severe intraocular inflammation (i.e., endophthalmitis, 4+ anterior chamber cell/flare, or 4+ vitreous cells).

All of the above-listed sight-threatening adverse events were reported as serious adverse events, listing the underlying cause (if known) of the event as the primary event term.

### H. Efficacy

The efficacy of AAV2.7m8-aflibercept in the treatment of DME was assessed by the following measures. Baseline values for BCVA and SD-OCT endpoints referred to pre-treatment measurements taken on the Day 1 visit when aflibercept IVT or sham ocular injection were administered.
BCVA: Vision was assessed primarily through BCVA expressed as an ETDRS score (number of letters correctly read). Maintenance of vision was classified if the subject has lost fewer than 15 letters in the ETDRS score compared to Baseline. Calculated endpoints included the mean change from Baseline, the percent gaining at least 15 letters compared to Baseline, and the percent losing 15 or more letters compared to Baseline.
Central subfield thickness: SD-OCT was performed using approved equipment and standard techniques to evaluate thickness and fluid compared to Baseline values. Endpoints included CST and macular volume.
Aflibercept re-treatments: The incidence and timing of aflibercept injections given post-AAV2.7m8-aflibercept treatment over time.
For each timepoint, Diabetic Retinopathy Severity Scale (DRSS) was determined using ultra-wide field color fundus photography and compared to Day 1.
Vision threatening complications (anterior segment neovascularization, diabetic macular edema, high-risk PDR development, vitreous hemorrhage, or tractional retinal detachment), as determined by ultra-wide field imaging and clinical examination.

### I. Statistical Analyses

The main analysis population included all randomized subjects who received the study treatment on Day 8 (AAV2.7m8-aflibercept IVT or Sham ocular injection). All safety and efficacy variables were summarized descriptively by treatment arm. Mean, standard deviation (SD), median and range were provided for continuous variables; and frequency counts and percentages were provided for categorical variables. Confidence intervals of the means and percentages were provided at both 90% and 95% levels. Kaplan-Meier survival analysis was utilized to derive median time to the first occurrence of DME disease worsening. All rescue aflibercept (2 mg IVT) received by each subject during the study were summarized using statistical models for recurrent events. Mean cumulative function (MCF) curve over time was plotted for the mean cumulative number of injections. Mixed-effect models for repeated measures (MMRM) were employed to explore the treatment effect on the change over time in BCVA and CST. The treatment effect on DRSS changes over time was explored using generalized mixed models for categorical outcomes. An interim analysis (IA) occurred after all subjects have been followed for 24 weeks.

### V. Results

One subject, a 56-year-old American Indian or Alaskan Native female with Diabetic Macular Edema (OU) in Arm 1 (e.g., who received treatment with a dose of 6E11 vector genomes of AAV2.7m8-aflibercept) experienced a serious adverse event of hypotony approximately 30 weeks after being randomized to study drug vs. sham in this study. Per the current Investigator Brochure (edition 4.0), hypotony is unexpected for this study drug. The patient remained on study and continued to be treated and followed closely. Initiation of steroid therapy was recommended if IOP ≤10 mmHg was observed, even in the absence of signs of clinical inflammation.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

1. A method for treating glaucoma in an individual, the method comprising administering a unit dose of recombinant adeno-associated virus (rAAV) particles to one eye of the individual, wherein the individual is a human, and wherein the rAAV particles comprise
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

2. The method of claim 1, wherein the glaucoma is neovascular glaucoma.

3. A method for reducing intraocular pressure in an individual, the method comprising administering a unit dose of recombinant adeno-associated virus (rAAV) particles to one eye of the individual, wherein the individual is a human, and wherein the rAAV particles comprise
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

3.a A method of reducing intraocular pressure in an individual, the method comprising administering a unit dose of rAAV particles to one eye of the individual, wherein the individual is a human, and wherein the rAAV particles comprise
a) a nucleic acid encoding a polypeptide under the control of a strong promoter that is active in the ciliary body and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

4. The method of claim 3, wherein the individual has glaucoma.

5. The method of claim 4, wherein the glaucoma is neovascular glaucoma.

6. The method of any one of claims 1-5, wherein the unit dose of rAAV particles is about 6×10¹¹ vector genomes per eye (vg/eye) or less.

7. The method of any one of claims 1-6, wherein the unit dose of rAAV particles is between about 6×10¹⁰ to about 6×10¹¹ vector genomes per eye (vg/eye).

8. The method of any one of claims 1-7, wherein the unit dose of rAAV particles is between about 6×10¹⁰ to about 2×10¹¹ vector genomes per eye (vg/eye).

9. The method of any one of claims 1-8, wherein the unit dose of rAAV particles is between about 2×10¹¹ to about 6×10¹¹ vector genomes per eye (vg/eye).

10. The method of claim 9, wherein the unit dose of rAAV particles is about 2×10¹¹ or about 6×10¹¹ vector genomes per eye (vg/eye).

11. The method claim 10, wherein the unit dose of rAAV particles is about 2×10¹¹ vector genomes per eye (vg/eye).

12. The method claim 11, wherein the unit dose of rAAV particles is about 6×10¹¹ vector genomes per eye (vg/eye).

13. The method of any one of claims 1-12, further comprising administering a unit dose of rAAV particles to the contralateral eye of the individual.

14. The method of claim 13, wherein the administering the unit dose of rAAV particles to the contralateral eye is up to about 2 weeks after administering the unit dose of rAAV particles to the one eye.

15. The method of claim 14, wherein:
(a) the administering the unit dose of rAAV particles to the contralateral eye is on the same day as the administering the unit dose of rAAV particles to the one eye; or
(b) the administering the unit dose of rAAV particles to the contralateral eye is between about 1 day to about 14 days after administering the unit dose of rAAV particles to the one eye.

16. The method of any one of claims 13-15, wherein the unit dose of rAAV particles administered to the contralateral eye of the individual comprises the same or less vector genomes per eye (vg/eye) than the unit dose of rAAV particles administered to the one eye of the individual.

17. The method of claim 16, wherein the administering the unit dose of rAAV particles to the contralateral eye is at least about 2 weeks after administering the unit dose of rAAV particles to the one eye.

18. The method of claim 15, wherein the unit dose of rAAV particles administered to the contralateral eye of the individual comprises more vector genomes per eye (vg/eye) than the unit dose of rAAV particles administered to the one eye of the individual.

19. The method of any one of claims 1-18, wherein the nucleic acid comprises the nucleic acid sequence of SEQ ID NO: 40 or a sequence having at least 85% identity thereto.

20. The method of any one of claims 1-19, wherein the polypeptide comprises the amino acid sequence of SEQ ID NO: 35.

21. The method of any one of claims 1-20, wherein the polypeptide comprises the amino acid sequence of SEQ ID NO: 41.

22. The method of any one of claims 1-21, wherein the polypeptide is aflibercept.

23. The method of any one of claims 1-22, wherein the nucleic acid further comprises a first enhancer region, a promoter region, a 5′UTR region, a second enhancer region, and a polyadenylation site.

24. The method of any one of claims 1-23, wherein the nucleic acid comprises, in the 5′ to 3′ order:
(a) a first enhancer region;
(b) a promoter region;
(c) a 5′UTR region;
(d) a nucleic acid encoding a polypeptide comprising an amino acid sequence with at least about 95% identity to the amino acid sequence of SEQ ID NO: 35;
(e) a second enhancer region; and
(f) a polyadenylation site;
and flanked by AAV2 inverted terminal repeats (ITRs).

25. The method of claim 23 or claim 24, wherein the first enhancer region comprises a CMV sequence comprising the sequence of SEQ ID NO: 22 or a sequence having at least 85% identity thereto.

26. The method of any one of claims 23-25, wherein the promoter region comprises a CMV sequence comprising the sequence of SEQ ID NO: 23 or a sequence having at least 85% identity thereto.

27. The method of any one of claims 24-26, wherein the nucleic acid encoding a polypeptide comprises the nucleic acid sequence of SEQ ID NO: 40 or a sequence having at least 85% identity thereto.

28. The method of any one of claims 24-27, wherein the polypeptide comprises the amino acid sequence of SEQ ID NO: 35 or a sequence having at least 95% identity thereto.

29. The method of any one of claims 24-28, wherein the polypeptide comprises the amino acid sequence of SEQ ID NO: 41 or a sequence having at least 95% identity thereto.

30. The method of any one of claims 24-29, wherein the polypeptide is aflibercept.

31. The method of any one of claims 23-30, wherein the 5′UTR region comprises, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto.

32. The method of any one of claims 23-31, wherein the second enhancer region comprises a full EES sequence comprising the sequence of SEQ ID NO: 26 or a sequence having at least 85% identity thereto.

33. The method of any one of claims 23-32, wherein the polyadenylation site comprises a HGH polyadenylation site comprising the sequence of SEQ ID NO: 27 or a sequence having at least 85% identity thereto.

34. The method of any one of claims 1-22, wherein the nucleic acid further comprises (a) a first enhancer region comprising a CMV sequence comprising the sequence of SEQ ID NO: 22 or a sequence having at least 85% identity thereto; (b) a promoter region, comprising a CMV sequence comprising the sequence of SEQ ID NO: 23 or a sequence having at least 85% identity thereto; (c) a 5′UTR region comprising, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto; (d) a second enhancer region comprising a full EES sequence comprising the sequence of SEQ ID NO: 26 or a sequence having at least 85% identity thereto; and (e) a HGH polyadenylation site comprising the sequence of SEQ ID NO: 27 or a sequence having at least 85% identity thereto.

35. The method of any one of claims 1-34, wherein the nucleic acid comprises the sequence of SEQ ID NO: 39 or a sequence having at least 85% identity thereto.

36. The method of any one of claims 1-35, wherein the AAV2 capsid protein comprises the amino acid sequence LALGETTRPA (SEQ ID NO: 1) inserted between positions 587 and 588 of the capsid protein, wherein the amino acid residue numbering corresponds to an AAV2 VP1 capsid protein.

37. The method of any one of claims 1-36, wherein the AAV2 capsid protein comprises the amino acid sequence LGETTRP (SEQ ID NO: 14) inserted between positions 587 and 588 of the AAV2 VP1 comprising the sequence of SEQ ID NO: 13.

38. The method of any one of claims 1-37, wherein the AAV2 capsid protein comprises the amino acid sequence LALGETTRPA (SEQ ID NO: 1) inserted between positions 587 and 588 of the AAV2 VP1 comprising the sequence of SEQ ID NO: 13.

39. The method of any one of claims 1-36, wherein the rAAV particles comprise an AAV2 VP1 capsid protein comprising a GH loop that comprises the amino acid sequence of SEQ ID NO: 38 or an amino acid sequence having at least 90% sequence identity to SEQ ID NO: 38.

40. The method of any one of claims 1-39, wherein the administration of the unit dose of rAAV particles to the one eye and/or the contralateral eye is by intravitreal administration.

41. The method of any one of claims 1-40, wherein the unit dose of rAAV particles is in a pharmaceutical formulation.

42. The method of claim 41, wherein the pharmaceutical formulation comprises the rAAV particles, sodium chloride, sodium phosphate and a surfactant.

43. The method of claim 42, wherein the pharmaceutical formulation comprises about 150 to about 200 mM sodium chloride, about 1 to about 10 mM monobasic sodium phosphate, about 1 to about 10 mM dibasic sodium phosphate, about 0.0005% (w/v) to about 0.005% (w/v) poloxamer 188, and about 6×10¹³ to about 6×10¹⁰ vector genomes (vg) per mL (vg/mL) of the rAAV particles, wherein the pharmaceutical formulation has a pH of about 7.0 to about 7.5.

44. The method of claim 43, wherein the pharmaceutical formulation comprises about 180 mM sodium chloride, about 5 mM monobasic sodium phosphate, about 5 mM dibasic sodium phosphate, about 6×10¹² vg/mL of the rAAV particles, and about 0.001% (w/v) poloxamer 188, wherein the pharmaceutical formulation has a pH of about 7.3.

45. The method of claim 43, wherein the pharmaceutical formulation comprises about 180 mM sodium chloride, about 5 mM monobasic sodium phosphate, about 5 mM dibasic sodium phosphate, about 2×10¹² vg/mL of the rAAV particles, and about 0.001% (w/v) poloxamer 188, wherein the pharmaceutical formulation has a pH of about 7.3.

46. The method of claim 43, wherein the pharmaceutical formulation comprises about 180 mM sodium chloride, about 5 mM monobasic sodium phosphate, about 5 mM dibasic sodium phosphate, about 6×10¹¹ vg/mL of the rAAV particles, and about 0.001% (w/v) poloxamer 188, wherein the pharmaceutical formulation has a pH of about 7.3.

47. The method of any one of claims 1-46, wherein the unit dose of rAAV particles administered to the one eye and/or to the contralateral eye in a volume of about 25 μL to about 250 μL.

48. The method of claim 47, wherein the unit dose of rAAV particles administered to the one eye and/or to the contralateral eye comprises a volume of about 100 L.

49. The method of claim 48, wherein the unit dose of rAAV particles administered to the one eye and/or to the contralateral eye comprises a volume of about 30 L.

50. The method of any one of claims 1-49, wherein the individual received prior treatment for the ocular neovascular disease with an anti-VEGF agent.

51. The method of claim 50, wherein the individual has received 1 or 2 injections of an anti-VEGF agent in the one eye and/or in the contralateral eye prior to administration of the rAAV particles in the one eye and/or in the contralateral eye.

52. The method of claim 50 or 51, wherein the anti-VEGF agent is aflibercept.

53. The method of any one of claims 1-49, wherein the individual has not received prior treatment for the ocular neovascular disease with an anti-VEGF agent.

54. The method of any one of claims 1-53, wherein the unit dose of rAAV particles is administered in combinations with administration of an anti-VEGF agent.

55. The method of claim 54, comprising administering the unit dose of rAAV particles to the one eye of the individual about 1 week or about 7 days after administration of the anti-VEGF agent.

56. The method of claim 54 or claim 55, comprising administering the anti-VEGF agent to the one eye of the individual on Day 1, and administering the unit dose of rAAV particles to the one eye of the individual on Day 8.

57. The method of any one of claims 54-56, wherein the anti-VEGF agent comprises aflibercept.

58. The method of claim 57, where the aflibercept is administered at a dose of about 2 mg by intravitreal injection.

59. The method of any one of claims 1-58, wherein the unit dose of rAAV particles is administered in combination with steroid treatment.

60. The method of claim 59, wherein the steroid treatment is a corticosteroid treatment.

61. The method of claim 59 or claim 60, wherein the steroid treatment is a systemic steroid treatment.

62. The method of any one of claims 59-61, wherein the steroid treatment is an oral steroid treatment.

63. The method of any one of claims 59-62, wherein the steroid treatment is a prednisone treatment.

64. The method of claim 59 or claim 60, wherein the steroid treatment is a topical steroid treatment.

65. The method of claim 64, wherein the steroid treatment is a difluprednate treatment.

66. The method of any one of claims 59-65, wherein the steroid is administered before, during and/or after administration of the unit dose of rAAV particles to the one eye and/or to the contralateral eye.

67. The method of any one of claims 64-66, wherein the topical steroid comprises difluprednate 0.05% at a dose of about 1 μg to about 3 μg.

68. The method of any one of claims 64-67, wherein the topical steroid comprises difluprednate 0.05% at a dose of about 2.5 μg.

69. A unit dose of about 6×10¹¹ vector genomes (vg) or less of recombinant adeno-associated virus (rAAV) particles for use in a method for treating glaucoma in an individual, the method comprising administering said unit dose to one eye of the individual, wherein the individual is a human, and wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

70. A unit dose of rAAV particles for use in a method for reducing intraocular pressure in an eye of an individual in need thereof, the method comprising administering said unit dose to one eye of the individual, wherein the individual is a human, and wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

71. The unit does of claim 70, wherein the individual has glaucoma.

72. The unit dose of claim 69 or 71, wherein the glaucoma is neovascular glaucoma.


SEQUENCE LISTING (41 sequences)

SEQ ID NO: 1 - protein, 10 aa, Artificial Sequence
  Synthetic Construct
LALGETTRPA

SEQ ID NO: 2 - protein, 10 aa, Artificial Sequence
  Synthetic Construct
LANETITRPA

SEQ ID NO: 3 - protein, 10 aa, Artificial Sequence
  Synthetic Construct
LAKAGQANNA

SEQ ID NO: 4 - protein, 10 aa, Artificial Sequence
  Synthetic Construct
LAKDPKTTNA

SEQ ID NO: 5 - protein, 7 aa, Artificial Sequence
  Synthetic Construct
KDTDTTR

SEQ ID NO: 6 - protein, 7 aa, Artificial Sequence
  Synthetic Construct
RAGGSVG

SEQ ID NO: 7 - protein, 7 aa, Artificial Sequence
  Synthetic Construct
AVDTTKF

SEQ ID NO: 8 - protein, 7 aa, Artificial Sequence
  Synthetic Construct
STGKVPN

SEQ ID NO: 9 - protein, 10 aa, Artificial Sequence
  Synthetic Construct
LAKDTDTTRA

SEQ ID NO: 10 - protein, 10 aa, Artificial Sequence
  Synthetic Construct
LARAGGSVGA

SEQ ID NO: 11 - protein, 10 aa, Artificial Sequence
  Synthetic Construct
LAAVDTTKFA

SEQ ID NO: 12 - protein, 10 aa, Artificial Sequence
  Synthetic Construct
LASTGKVPNA

SEQ ID NO: 13 - protein, 735 aa, Adeno-associated virus [sequence omitted]

SEQ ID NO: 14 - protein, 7 aa, Artificial Sequence
  Synthetic Construct
LGETTRP

SEQ ID NO: 15 - protein, 7 aa, Artificial Sequence
  Synthetic Construct
NETITRP

SEQ ID NO: 16 - protein, 7 aa, Artificial Sequence
  Synthetic Construct
KAGQANN

SEQ ID NO: 17 - protein, 7 aa, Artificial Sequence
  Synthetic Construct
KDPKTTN

SEQ ID NO: 18 - protein, 7 aa, Artificial Sequence
  Synthetic Construct
KDTDTTR

SEQ ID NO: 19 - protein, 7 aa, Artificial Sequence
  Synthetic Construct
RAGGSVG

SEQ ID NO: 20 - protein, 7 aa, Artificial Sequence
  Synthetic Construct
AVDTTKF

SEQ ID NO: 21 - protein, 7 aa, Artificial Sequence
  Synthetic Construct
STGKVPN

SEQ ID NO: 22 - DNA, 293 bp, Artificial Sequence
  Synthetic Construct
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattacca

SEQ ID NO: 23 - DNA, 220 bp, Artificial Sequence
  Synthetic Construct
tgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 24 - DNA, 318 bp, Artificial Sequence
  Synthetic Construct
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcga

SEQ ID NO: 25 - DNA, 102 bp, Artificial Sequence
  Synthetic Construct
ccagctgttggggtgagtactccctctcaaaagcgggcattacttctgcgctaagattgtcagtttccaaaaacgaggaggatttgatattcacctggcccg

SEQ ID NO: 26 - DNA, 810 bp, Artificial Sequence
  Synthetic Construct
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 27 - DNA, 202 bp, Artificial Sequence
  Synthetic Construct
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 28 - DNA, 68 bp, Artificial Sequence
  Synthetic Construct
aggcggtcttgagacggcggatggtcgaggtgaggtgtggcaggcttgagatccagctgttggggtga

SEQ ID NO: 29 - DNA, 129 bp, Artificial Sequence
  Synthetic Construct
cgctgttttgacctccatagtggacaccgggaccgatccagcctccgcgtctcaggggagatctcgtttagtgaaccgtcagatcctcactctcttccgcatcgctgtctgcgagggccagctgttggg

SEQ ID NO: 30 - DNA, 36 bp, Artificial Sequence
  Synthetic Construct
ttgatattcacctggcccgatctggccatacacttg

SEQ ID NO: 31 - DNA, 22 bp, Artificial Sequence
  Synthetic Construct
cccaggtccaagtttaaacgcc

SEQ ID NO: 32 - DNA, 77 bp, Artificial Sequence
  Synthetic Construct
tctttgggctttctatatacattttgaaatgaggttgacaagttacctaggaaaactgtcttcctgcccgggtggca

SEQ ID NO: 33 - DNA, 1172 bp, Artificial Sequence
  Synthetic Construct
ctctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatg

SEQ ID NO: 34 - DNA, 1043 bp, Artificial Sequence
  Synthetic Construct
actgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgaagacagtcag

SEQ ID NO: 35 - protein, 431 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - DNA, 1377 bp, Artificial Sequence
  Synthetic Construct
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 745 aa, Artificial Sequence
  Synthetic Construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - protein, 250 aa, Artificial Sequence
  Synthetic Construct
FSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKN

SEQ ID NO: 39 - DNA, 3916 bp, Artificial Sequence
  Synthetic Construct
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggtgtcatactgggatactggagtcttgctttgtgccctgctgtcctgcctcctcctgactggctccagctcgggctcagataccggtcgccccttcgtggagatgtactccgagatcccggaaattatccacatgactgaggggcgcgaacttgtgatcccctgccgggtcaccagcccgaacattactgtgactttgaagaagttccccctggacaccctgattccggatgggaagagaattatctgggattcacggaagggattcatcatcagcaacgcgacctacaaggaaattggcctcctcacttgcgaagccactgtgaacggacacttgtacaagaccaactacctgacccaccgccagaccaacaccatcatcgacgtcgtcctgtccccttcgcacgggatcgagctctcggtgggagagaagttggtgcttaactgcaccgcccggacggaactgaatgtgggaatcgacttcaactgggaatacccgtccagcaagcatcagcataagaagctggtgaaccgggacctcaagactcagtccggcagcgaaatgaagaagttcctgtcgaccctcactattgacggagtgaccagatccgaccagggcctctacacttgcgccgcttccagcggactcatgaccaagaagaacagcactttcgtgagggtgcatgagaaggacaagacccacacgtgtccgccgtgcccagccccagagctgctgggaggcccttccgtgttcctgtttccgcccaagccaaaggataccctgatgatctcaaggacccctgaggtcacatgcgtcgtggtggatgtgtcgcacgaggaccctgaagtcaaattcaattggtatgtggacggagtggaagtccacaacgcgaaaaccaagccgagagaagaacagtacaattccacctaccgggtggtgtcggtgctgactgtgctgcaccaggactggctcaacggaaaggagtacaagtgcaaggtgtccaacaaggctctgcccgcacctattgaaaagaccatctccaaggccaagggtcaacctcgcgagcctcaggtgtacactctgcctccaagccgggacgaactgactaagaaccaagtctctctgacctgtttggtgaagggcttctacccgtcagacatcgcagtggagtgggagtcaaacggtcagccggagaacaactacaaaacaaccccccccgtgctggactccgacggctccttcttcctgtactccaagcttaccgtggataagagccgctggcaacagggcaacgtgttttcctgctccgtcatgcacgaagccctgcacaaccattatacccagaagtccctgtcgctgtcccccgggaaatagtgactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 40 - DNA, 1377 bp, Artificial Sequence
  Synthetic Construct
atggtgtcatactgggatactggagtcttgctttgtgccctgctgtcctgcctcctcctgactggctccagctcgggctcagataccggtcgccccttcgtggagatgtactccgagatcccggaaattatccacatgactgaggggcgcgaacttgtgatcccctgccgggtcaccagcccgaacattactgtgactttgaagaagttccccctggacaccctgattccggatgggaagagaattatctgggattcacggaagggattcatcatcagcaacgcgacctacaaggaaattggcctcctcacttgcgaagccactgtgaacggacacttgtacaagaccaactacctgacccaccgccagaccaacaccatcatcgacgtcgtcctgtccccttcgcacgggatcgagctctcggtgggagagaagttggtgcttaactgcaccgcccggacggaactgaatgtgggaatcgacttcaactgggaatacccgtccagcaagcatcagcataagaagctggtgaaccgggacctcaagactcagtccggcagcgaaatgaagaagttcctgtcgaccctcactattgacggagtgaccagatccgaccagggcctctacacttgcgccgcttccagcggactcatgaccaagaagaacagcactttcgtgagggtgcatgagaaggacaagacccacacgtgtccgccgtgcccagccccagagctgctgggaggcccttccgtgttcctgtttccgcccaagccaaaggataccctgatgatctcaaggacccctgaggtcacatgcgtcgtggtggatgtgtcgcacgaggaccctgaagtcaaattcaattggtatgtggacggagtggaagtccacaacgcgaaaaccaagccgagagaagaacagtacaattccacctaccgggtggtgtcggtgctgactgtgctgcaccaggactggctcaacggaaaggagtacaagtgcaaggtgtccaacaaggctctgcccgcacctattgaaaagaccatctccaaggccaagggtcaacctcgcgagcctcaggtgtacactctgcctccaagccgggacgaactgactaagaaccaagtctctctgacctgtttggtgaagggcttctacccgtcagacatcgcagtggagtgggagtcaaacggtcagccggagaacaactacaaaacaaccccccccgtgctggactccgacggctccttcttcctgtactccaagcttaccgtggataagagccgctggcaacagggcaacgtgttttcctgctccgtcatgcacgaagccctgcacaaccattatacccagaagtccctgtcgctgtcccccgggaaatag

SEQ ID NO: 41 - protein, 458 aa, Artificial Sequence
  Synthetic Construct
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK